(19) United States
(12) Patent Application Publication
(10) Pub. No.: US 2025/0188115 A1
(43) Pub. Date: Jun. 12, 2025

(54) PROCESS FOR PREPARING A GLUCAGON-LIKE PEPTIDE

(71) Applicant: CHEMICAL & BIOPHARMACEUTICAL LABORATORIES OF PATRAS S.A., Patras (GR)

(72) Inventors: Kleomenis BARLOS, Patras (GR); Konstantinos BARLOS, Patras (GR); Dimitrios GATOS, Patras (GR); Zoi VASILEIOU, Patras (GR)

(21) Appl. No.: 18/766,010

(22) Filed: Jul. 8, 2024

Related U.S. Application Data
(63) Continuation of application No. 16/652,910, filed on Apr. 1, 2020, now abandoned.

(30) Foreign Application Priority Data
Oct. 4, 2017 (GR) 20170100453
Oct. 3, 2018 (GR) 20180100447

(51) Int. Cl. C07K 1/02; C07K 14/605

(52) U.S. Cl. CPC C07K 1/02; C07K 14/605

(57) ABSTRACT

A process for preparing a GLP-1 or GLP-2 peptide, said process comprising coupling in solution at least a first fragment and at least a second fragment, wherein the coupling comprises reacting the carboxy terminal amino acid of the first fragment with the amino terminal amino acid of the second fragment, and wherein the carboxy terminal amino acid of the first fragment is other than a Gly residue

### RELATED APPLICATIONS

This application is a Continuation of U.S. patent application Ser. No. 16/652,910, filed Apr. 1, 2020, which application is a 35 U.S.C. § 371 filing of International Patent Application No. PCT/IB2018/057736, filed Oct. 4, 2018, which claims the benefit of Greek patent application Ser. No. 20/170,100453, filed Oct. 4, 2017, and Greek patent application Ser. No. 20/180,100447, filed Oct. 3, 2018, the contents of each of which are incorporated herein by reference in their entirety.

### SEQUENCE LISTING

The instant application contains a Sequence Listing, which has been submitted electronically, and is hereby incorporated by reference in its entirety. Said XML copy, created on Feb. 27, 2025, is named “756124_DYT-029USCON_SL_2.xml” and is 650,584 bytes in size.

### FIELD OF THE INVENTION

The present invention describes a process for the synthesis of glucagon-like peptides and analogues and variants thereof, more specifically, the GLP-1 peptide analogues Liraglutide and Semaglutide, and the GLP-2 peptide analogue Teduglutide. The method is based on the condensation of two or more fragments in solution.

### BACKGROUND OF THE INVENTION

The glucagon-like peptides (GLP-1 and GLP-2) are processed from the proglucagon polypeptide in the gut. GLP-2 is co-encoded with GLP-1 inside the pro-glucagon gene, and secreted in a 1:1 ratio with GLP-1 from intestinal cells [Endocrinology; 1986 October; 119(4):1467-75]. GLP-1 and GLP-2 are co-secreted in equimolar amounts upon nutrient ingestion, but have opposite effects on chylomicron (CM) production, with GLP-1 significantly reducing and GLP-2 increasing postprandial chylomicronemia.

Glucagon-like peptide-2 (GLP-2) is a 33 amino acid peptide with the sequence (in humans):

“GLP-2”
H-His-Ala-Asp-Gly-Ser-Phe-Ser-Asp-Glu-Met-Asn-Thr-
Ile-Leu-Asp-Asn-Leu-Ala-Ala-Arg-Asp-Phe-Ile-Asn-
Trp-Leu-Ile-Gln-Thr-Lys-Ile-Thr-Asp-OH

GLP-2 is created by specific post-translational proteolytic cleavage of proglucagon in a process that also liberates the related glucagon-like peptide-1 (GLP-1). GLP-2 is produced by the intestinal endocrine L cell and by various neurons in the central nervous system.

When externally administered, GLP-2 produces a number of effects in humans and rodents, including intestinal growth, enhancement of intestinal function, reduction in bone breakdown and neuroprotection. GLP-2 may act in an endocrine fashion to link intestinal growth and metabolism with nutrient intake. GLP-2 and related analogues may be treatments for short bowel syndrome, Crohn's disease, osteoporosis and as adjuvant therapy during cancer chemotherapy.

Teduglutide is a 33-membered polypeptide and GLP-2 analogue having the sequence:

Teduglutide
H-His-Gly-Asp-Gly-Ser-Phe-Ser-Asp-Glu-Met-Asn-Thr-
Ile-Leu-Asp-Asn-Leu-Ala-Ala-Arg-Asp-Phe-Ile-Asn-
Trp-Leu-Ile-Gln-Thr-Lys-Ile-Thr-Asp-OH

Teduglutide is used for the treatment of short bowel syndrome and works by promoting mucosal growth and possibly restoring gastric emptying and secretion [“Teduglutide, a novel glucagon-like peptide 2 analog, in the treatment of patients with short bowel syndrome”; Therap Adv Gastroenterol. 5 (3): 159-71].

Glucagon-like peptide-1 (GLP-1) is a 30 amino acid peptide hormone derived from the tissue-specific post-translational processing of the proglucagon gene, having the sequence:

“GLP-1”
H-His-Ala-Glu-Gly-Thr-Phe-Thr-Ser-Asp-Val-Ser-Ser-
Tyr-Leu-Glu-Gly-Gln-Ala-Ala-Lys-Glu-Phe-Ile-Ala-
Trp-Leu-Val-Lys-Gly-Arg-NH₂.

GLP-1 is produced and secreted by intestinal enteroendocrine L-cells and certain neurons within the nucleus of the solitary tract in the brainstem upon food consumption. The initial product GLP-1 (1-37) is susceptible to amidation and proteolytic cleavage which gives rise to the two truncated and equipotent biologically active forms, GLP-1 (7-36) amide and GLP-1 (7-37). Active GLP-1 composes two α-helices from amino acid position 13-20 and 24-35 separated by a linker region

GLP-1 possesses several physiological properties making it (and its functional analogues) a subject of intensive investigation as a potential treatment of diabetes mellitus. These properties include the potentiation of the glucose-induced secretion of insulin, increased insulin expression, inhibition of the apoptosis of beta-cells, the reduction of glucagon secretion, and the promotion of satiety. These advantageous properties have prompted intensive research and development into several therapeutic GLP-1 analogues, including Liraglutide and Semaglutide.

Liraglutide is a GLP-1 peptide analogue having a 31 amino acid backbone with the structure shown below:

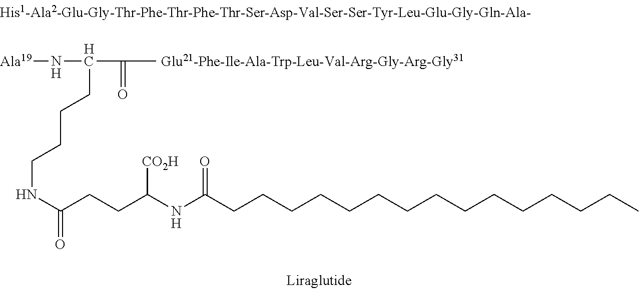

Semaglutide shares a similar backbone to Liraglutide, but differs in the amino acid in the 2-position (Aib in Semglutide; Ala in Liraglutide) and in the modification of the Lys²⁰ side chain.

The Lys²⁰ in Liraglutide is modified with 2-palmitamidopentanedioic acid

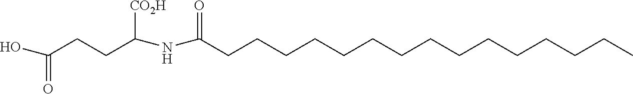 [SMILES: CCCCCCCCCCCCCCCC(=O)NC(CCC(=O)O)C(=O)O]

whereas in Semaglutide, it is modified with 9,18,23-trioxo-2,5,11,14-tetraoxa-8,17,22-triazanonatriacontane-1,21,39-tricarboxylic acid.

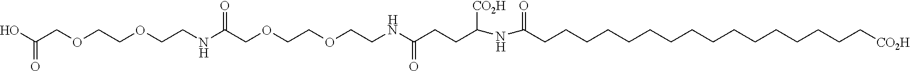 [SMILES: O=C(O)CCCCCCCCCCCCCCCCC(=O)NC(CCC(=O)NCCOCCOCC(=O)NCCOCCOCC(=O)O)C(=O)O]

Semaglutide has the structure shown below:

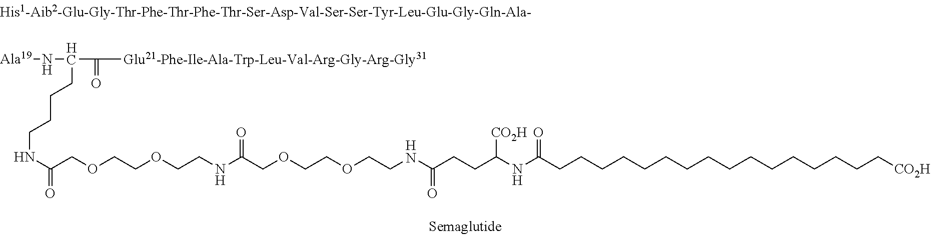

Liraglutide and Semaglutide are used for the treatment of dibetes type 2, obesity and also have promising applications in the treatment of Alzheimer's disease.

In the step-by-step solid-phase synthesis of GLP-1 analogues, Liraglutide and Semaglutide are known to contain positions in their sequences where the coupling and deprotection steps become difficult. This results in the formation of several undesirable byproducts which lower the yield and make purification more difficult. Similar difficulties apply in relation to the synthesis of Teduglutide. Several methods have been developed in an attempt to overcome these disadvantages. However, to date, none have proved completely satisfactory.

One particular difficulty with the synthesis lies in the incorporation of Lys(20) into the growing peptide chain of Liraglutide and Semaglutide. Indeed, difficulties arise both before and after modification of the side chain. Especially after Lys incorporation, the coupling reactions become very slow, resulting in racemization and the formation of failure sequences (i.e. those having an amino acid less or more) in a higher degree than expected.

The synthesis of Liraglutide is described in U.S. Pat. Nos. 6,268,343, 6,458,924 and 6,451,974. Recombinant DNA synthesis is used to prepare the peptide intermediate (1-31), which contains two free amino groups, one at the N-terminus, and the other in the side chain of Lys(20). The Palmitoyl-glutamyl (Pal-Glu) unit is then introduced into the Lys side chain. However, this coupling reaction cannot be performed with high selectivity at the Lys side chain amino group, and gives rise to the formation of N-alpha-Pal-Glu-GLP-1(1-31), as well as the doubly acylated form. These side products, together with the complexity of using recombinant DNA technique for large scale synthesis, considerably lowers the yield and complicates purification of the desired GLP-1 analogues.

U.S. Pat. No. 8,445,433 describes a method of synthesizing GLP-1 analogues by step-by-step solid phase synthesis using Fmoc-pseudoproline dipeptides for the assembly of the peptide chain instead of Fmoc-amino acids. Similarly, WO 2007/090496 discloses a method of synthesizing GLP-1 peptide agonists by linear sequential synthesis, using an Fmoc-pseudoproline dipeptide unit at the relevant position in order to prepare the Val-Ser or Ser-Ser segment of the peptide chain. The remaining sequence is then prepared by stepwise sequential synthesis. Pseudoprolines are known to improve the reaction rates in difficult regions, for example, where β-turns and β-sheets prevent the reagents from reaching the required reaction sites, thereby leading to the formation of failure sequences (Sampson W. R. et al: “The synthesis of ‘difficult’ peptides using 2-hydroxy-4-methoxybenzyl or pseudoproline amino acid building blocks: A comparative study” Journal of Peptide Science, vol. 5, no. 9, 1999, pages 403-409).

CN 102286092 A and EP20120831927 describe the solid phase sequential synthesis of Liraglutide using Fmoc-Lys(Alloc)-OH. After the Liraglutide chain is assembled, the Alloc-group is removed catalytically using Pd(PPh₃)₄ and Pal-Glu-O^{t}Bu is reated with the liberated Lys side chain, before deprotection and cleavage from the resin. However, Pd(PPh₃)₄ is unsuitable for large scale synthesis in view of its toxicity, handling difficulties and high cost. Moreover, it is difficult to assemble full length peptides in an acceptable yield and purity using sequential solid phase synthesis.

CN 103145828 describes the solid phase synthesis of Liraglutide using Fmoc-Lys(ivDde). After the Liraglutide chain is assembled, the Dde-group is removed by hydrazine treatment of the resin-bound peptide. Pal-Glu-O^{t}Bu is then reacted with the liberated Lys side chain, before deprotection cleavage from the resin. However, hydrazines are unsuitable for large scale synthesis in view of their toxicity, and the formation of side products caused by the cleavage of sensitive peptide bonds and/or the hydrolysis of amide bonds.

CN 103864918 discloses the solid phase synthesis of Liraglutide by coupling a fragment containing amino acid residues (1-10) with a fragment containing amino acid residues (11-31), removing the resin and protecting groups, before purifying and freeze drying the resulting product. The Lys residue is incorporated using Fmoc-Lys (Mtt)-OH, with the Mtt side chain being removed selectively in the presense of the (21-31) resin bound peptide before introduction of the Pal-Glu group. The resulting (20-31)-Pal-Glu fragment—still bound on the Wang resin—is condensed with the (11-19) fragment, and then with the (1-10) fragment. Alternatively, the (1-10) fragment is condensed first with the (11-19) fragment, and the resulting (1-19) fragment is then condensed with the resin-bound (20-31) fragment. The peptide is then deprotected and cleaved from the resin. However, this synthesis has several important drawbacks. Firstly, peptide fragment condensations on the resin are slow, which necessitates the use of an excess of costly peptide fragments in order to allow the condensation to proceed with an acceptable speed. Secondly, because fragment condensation is slow, racemization occurs if the C-terminal amino acid is not Gly or Pro, and increases considerably with the prolongation of the condensation time. Thus, extensive racemization is expected if the fragments (1-10) and (11-19) are used for fragment condensation on the resin. Thirdly, Val is the most sterically hindered amino acid. Thus, if Val is used for fragment condensation on the resin, very slow condensation rates and extensive racemization are expected, more so than for any other amino acid contained in the Liraglutide sequence. Fourthly, the Pal-Glu on-resin derivatization also requires an excess of activated Pal-Glu derivative to be used in order to drive the reaction to completion. As this derivative is costly, this again is a significant drawback to the synthesis. Fifthly, the removal of the Mtt function requires the use of 5% TFA, which causes the partial removal of side chain protecting groups such as ^{t}Bu, Boc and Pbf functions. In turn, this causes the formation of further side products. Finally, resin-bound alkoxybenzyl cations formed during the cleavage of the peptide from the Wang resin and the deprotection react and bind irreversibly to resin peptides containing Trp in their peptide chain, which significantly reduces the yield of the desired peptide.

CN 104004083 discloses the solid phase synthesis of Liraglutide from peptide fragments containing amino acid residues (1-4), (15-16) and (17-31). More specifically, the method involves coupling the (15-16) fragment with the (17-31) fragment, and sequentially adding amino acids thereto before coupling with the (1-4) fragment, removing the resin and protecting groups, and purifying the resulting product. However, this synthetic approach requires an excess of costly fragments to drive the condensation reactions on the resin to completion. Moreover, it also requires an excess of the costly activated Pal-Glu derivative to modify the side chain of the Lys residue. Furthermore, the conditions required for removal of the Mtt function and detachment from the resin lead to the same difficulties outlined above in relation to CN 103145828.

WO 2016/046753 discloses methods for synthesizing GLP-1 peptides, including Liraglutide and Semaglutide, which comprise a final coupling step in which at least two fragments are coupled at a terminal Gly residue, wherein at least one of the fragments is prepared by the coupling of at least two sub-fragments. By way of example, WO 2016/046753 discloses coupling fragment (1-4) and fragment (5-31) in solid state or in solution. Fragment (5-31) can be prepared by coupling fragment (5-16) with fragment (17-31). Fragment (5-16) itself can be prepared by coupling fragment (5-12) with fragment (13-16). Coupling with a terminal Gly, for example, at Gly4 or Gly16, avoids racemization.

A method for the preparation of Teduglutide is described in CN104817638 and involves synthesizing fragments (1-2), (3-4) and (5-33) and coupling said fragments together. CN104418949 describes the synthesis of Teduglutide from fragments (1-3) and (4-33). CN104072603 descrbes the synthesis of Teduglutide by coupling a His residue with a fragment (2-33). CN104072605 describes the synthesis of Teduglutide from an Asp-Gly dipeptide starting material by preparing a C-end fragment and a middle fragment. Further methods for preparing Teduglutide are described in CN106749614.

To date, none of the existing methods for the bulk production of GLP-1 peptides (such as Liraglutide and Semaglutide) and GLP-2 peptides (such as Tedulglutide) are completely satisfactory. The present invention therefore seeks to provide alternative methods for the synthesis of GLP-1 and GLP-2 peptides, ideally methods that are more efficient, and lead to improved yields and/or purity. In particular, there is a need to provide methods that are suitable for industrial scale-up, and which avoid the use of toxic or otherwise undesirable reagents.

### STATEMENT OF INVENTION

A first aspect of the invention relates to a process for preparing a GLP peptide or an analogue or variant thereof, said process comprising coupling in solution at least a first fragment and at least a second fragment, wherein the coupling comprises reacting the carboxy terminal amino acid of the first fragment with the amino terminal amino acid of the second fragment, and wherein the carboxy terminal amino acid of the first fragment is other than a Gly residue.

The present invention enables the coupling of the peptide fragments in solution, i.e. without the need for a hydrophobic solid support. The Applicant has synthesized various fragments of Liraglutide and Semaglutide with C-terminal amino acids other than Gly. Specifically, the fragments (1-19), (1-18), (1-17) were synthesized and condensed with the corresponding fragments (20-31), (19-31), (18-31) in a solution phase reaction. It was expected that the fragments would racemize to give a mixture of D- and L-diastereomeric peptides at the condensation positions (for example, DL-Ala(19) Liraglutide, DL-Ala(19) Semaglutide, DL-Ala(18) Liraglutide, DL-Ala(18) Semaglutide, DL-Gln(17) etc). This is because it is well established in the art that protected fragments containing amino acids other than Gly or Pro at the C-terminal racemize extensively during their condensation. However, contrary to expectations, studies by the Applicant showed that certain fragments led to unexpectedly low levels of racemization. Similar results were observed for the synthesis of Teduglutide using fragments (1-14) and (15-33), (1-17) and (18-33), (1-18) and (19-33), and (1-19) and (20-33).

In another approach for preparing Liraglutide/Semaglutide, smaller fragments were synthesized (such as the (2-19), (3-19) . . . (17-19) fragments) and after condensation with the (20-31) fragment in solution, the synthesis was continued by the step by step method, or by fragment condensation, to obtain Liraglutide or Semaglutide. Similar observations were made in relation to the lower than expected levels of racemization.

For example, solution phase coupling of fragment (20-31) with fragment (1-19) or fragment (2-19) racemized to a very low extent (<5%).

### DETAILED DESCRIPTION

The present invention relates to a process for preparing a GLP peptide or an analogue or variant thereof, said process comprising coupling in solution at least a first fragment and at least a second fragment, wherein the coupling comprises reacting the carboxy terminal amino acid of the first fragment with the amino terminal amino acid of the second fragment, and wherein the carboxy terminal amino acid of the first fragment is other than a Gly residue.

The present synthetic approach allows introduction of the Pal-Glu unit (for Liraglutide) and the N-(17-carboxy-1-oxoheptadecyl)-L-y-glutamyl-2-[2-(2-aminoethoxy) ethoxy]acetyl-2-[2-(2-aminoethoxy)ethoxy]acetyl (for Semaglutide) on the side chain of Lys at an early stage of the synthesis.

Resin-bound fragments (20-31), (19-31), (18-31) etc were synthesized by the step by step method on 2-chlorotrityl resin or the Wang resin using Fmoc-Lys²⁰(Pal-Glu-OtBu)-OH or Fmoc-Lys(C18-Glu-PEG2)-OH. Alternatively, the fragments were prepared by introducing Lys(20) with Fmoc-Lys(Mmt)-OH on 2-chlorotrityl resin. The fragments were then cleaved from the resin with simultaneous removal of the Mmt group from the side chain of Lys(20). Pal-Glu or C18-Glu-PEG2 was then incorporated using Fmoc or C18-Glu(OSu)-PEG2 or the corresponding Pfp derivatives. The formed Lys²⁰(Pal-Glu-OtBu)- or Lys²⁰(C18Glu-PEG2)-(20-31) fragments were then esterified in solution using a suitable method with a trityl type group, a diphenyl methyl type group or with ^{t}Bu.

The protected esters (20-31, 19-31, 18-31 etc) were then condensed in solution with the L-Ala(1-19), L-Ala(1-18), L-Gln(1-17) protected fragments using methods known in the art. Preferably, dehydrating agents such as EDAC/DIPEA, DIC, HBTU and an acidic catalyst such as HOBt, HOAtu, PfpOH are used to facilitate the condensation reaction. Solid phase couplings were also carried out for comparison.

As expected, the condensations performed on solid-phase, using either the Wang or the 2-chlorotrityl resin proceeded with extensive racemization. In this regard, it was determined by HPLC that >33% D-isomer was formed in all cases, even though the condensation conditions were chosen to be as mild as possible. Lowering the condensation temperature led to a similar degree of racemization to when the condensation was performed at room temperature. Without wishing to be bound by theory, it is believed that at lower temperatures, the condensation becomes very slow so that the racemization increases concurrently.

However, surprisingly, when carried out in solution, the degree of racemization proved to be much lower (<7%) than expected compared to the condensation results on solid phase. In particular, at the positions Ala(19), Ala(20), Gln(18), and Leu(14), the racemization was observed to be less than 3%.

In one preferred embodiment, the racemization at the coupling position between the first and second fragments is less than 10%, more preferably less than 9%, more preferably less than 8%, more preferably less than 7%, more preferably less than 6%, more preferably less than 5%, more preferably less than 4%, more preferably less than 3%.

In one preferred embodiment, the carboxyl terminal amino acid of the first fragment is an Ala residue. For example, for Liraglutide/Semaglutide, the process comprises coupling a fragment (1-19) with a fragment (20-31), or a fragment (1-18) with a fragment (19-31).

In another preferred embodiment, the carboxyl terminal amino acid of the first fragment is a Gln residue. For example, for Liraglutide/Semaglutide, the process comprises coupling a fragment (1-17) with a fragment (18-31).

In another preferred embodiment, the carboxyl terminal amino acid of the first fragment is a Leu residue. For example, for Liraglutide/Semaglutide, the process comprises coupling a fragment (1-14) with a fragment (15-31).

In one preferred embodiment, the carboxy terminal residue of the first fragment is an amino acid ester or an amino acid amide. In an even more preferred embodiment, the amino acid ester group is selected from a trityl type group, a diphenylmethyl group and a tert-butyl group.

In addition to the specific peptides mentioned herein, the invention also encompasses variants, derivatives, analogues, homologues and fragments thereof.

As used herein, a “variant” of any given sequence is a sequence in which the specific sequence of amino acid residues has been modified in such a manner that the peptide in question retains at least one of its endogenous functions. A variant sequence can be obtained by addition, deletion, substitution, modification, replacement and/or variation of at least one residue present in the naturally occurring peptide.

The term “derivative” as used herein in relation to peptides described herein includes any substitution of, variation of, modification of, replacement of, deletion of and/or addition of one (or more) amino acid residues from or to the sequence, providing that the resultant peptide retains at least one of its endogenous functions.

The term “analogue” as used herein in relation to peptides includes any mimetic, that is, a chemical compound that possesses at least one of the endogenous functions of the peptides which it mimics.

Typically, amino acid substitutions may be made, for example from 1, 2 or 3, to 10 or 20 substitutions, provided that the modified sequence retains the required activity or ability. Amino acid substitutions may include the use of non-naturally occurring analogues.

Peptides described herein may also have deletions, insertions or substitutions of amino acid residues which produce a silent change and result in a functionally equivalent peptide. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar—uncharged | C S T M |
| | | N Q |
| | Polar—charged | D E |
| | | K R H |
| AROMATIC | | F W Y |

The term “homologue” as used herein means an entity having a certain homology with the wild type amino acid sequence. The term “homology” can be equated with “identity”.

In the present context, a homologous sequence is taken to include an amino acid sequence which may be at least 50%, 55%, 65%, 75%, 85% or 90% identical, preferably at least 95%, 96% or 97% or 98% or 99% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

Preferably, reference to a sequence which has a percent identity to any one of the SEQ ID NOs detailed herein refers to a sequence which has the stated percent identity over the entire length of the SEQ ID NO referred to.

Homology comparisons can be conducted by eye, or more usually, with the aid of readily available sequence comparison programs. These commercially available computer programs can calculate percent homology or identity between two or more sequences.

Percent homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the amino acid sequence may cause the following residues or codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids or nucleotides, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percent homology therefore firstly requires the production of an optimal alignment, taking into consideration gap penalties. A suitable computer program for carrying out such an alignment is the GCG Wisconsin Bestfit package (University of Wisconsin, USA; Devereux et al. (1984) Nucleic Acids Research 12:387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, BLAST 2 Sequences, is also available for comparing protein and nucleotide sequences (FEMS Microbiol. Lett. (1999) 174 (2): 247-50; FEMS Microbiol. Lett. (1999) 177 (1): 187-8).

Although the final percent homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix (the default matrix for the BLAST suite of programs). GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62.

Once the software has produced an optimal alignment, it is possible to calculate percent homology, preferably percent sequence identity. The software typically does this as part of the sequence comparison and generates a numerical result.

“Fragments” are also variants and the term typically refers to a selected region of the peptide that is of interest either functionally or, for example, in an assay. “Fragment” thus refers to an amino acid sequence that is a portion of a full-length peptide.

More preferably, the term “variant” includes any variation wherein wherein (a) one or more amino acid residues are replaced by a naturally or non-naturally occurring amino acid residue (b) the order of two or more amino acid residues is reversed, (c) one, two or three amino acids are deleted, (d) a spacer group is present between any two amino acid residues, (e) one or more amino acid residues are in peptoid form, (f) the (N—C—C) backbone of one or more amino acid residues of the peptide has been modified, (g) one or more additional amino acids are present at the N-terminus and/or the C-terminus, or any of (a)-(g) in combination. Preferably, the variants arise from one of (a), (b) or (c).

The present invention also encompasses amino acid sequences modified by the incorporation of one or more pseudoprolines (denoted ψ). Pseudoprolines are artificially created dipeptides that minimize aggregation during FMOC solid phase synthesis of peptides. Pseudoprolines consist of serine—(Oxa) or threonine-derived oxazolidines [Oxa (5-Me)] and Cysteine-derived thiazolidines (THz) with Proline-like ring structures (see below).

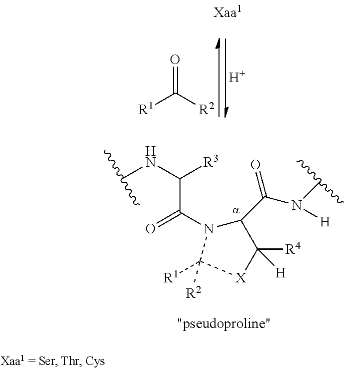

Due to the preference for a cis-amide bond with the preceding residue of C2-substituted pseudoprolines, their incorporation results in a kink conformation of the peptide backbone, thereby preventing peptide aggregation, self-association, or β-structure formation. Hence, pseudoprolines fulfil two functions simultaneously: firstly, they serve as temporary side-chain protection for Ser, Thr, and Cys, and secondly they act as solubilizing building blocks to increase solvation and coupling rates during peptide synthesis and in subsequent chain assembly.

Pseudoprolines are obtained by reacting the free amino acids with aldehydes or ketones. Pseudoproline dipeptides can be introduced in the same manner as other amino acid derivatives. Preferably the pseudoproline is derived from a Ser-X, Thr-X or Cys-X group, where X is a natural or unnatural amino acid. The routine use of pseudoproline (oxazolidine) dipeptides in the FMOC solid phase peptide synthesis (SPPS) of serine-and threonine-containing peptides leads to significant improvements in quality and yield of crude products. Once the peptide is deprotected, the pseuoproline becomes a conventional dipeptide of the form X-Ser, X-Thr or X-Cys, wherein X is a natural or unnatural amino acid.

More preferably, the variant has one to five, or one to four, or one to three amino acids residues substituted by one or more other amino acid residues. Even more preferably, two amino acid residues are substituted by another amino acid residue. More preferably still, one amino acid residue is substituted by another amino acid residue. Preferably, the substitution is homologous.

Homologous substitution (substitution and replacement are both used herein to mean the interchange of an existing amino acid residue, with an alternative residue) may occur, i.e. like-for-like substitution such as basic for basic, acidic for acidic, polar for polar etc. Non-homologous substitution may also occur i.e. from one class of residue to another or alternatively involving the inclusion of unnatural amino acids such as ornithine, diaminobutyric acid ornithine, norleucine ornithine, pyridylalanine, thienylalanine, naphthylalanine and phenylglycine, a more detailed list of which appears below. More than one amino acid residue may be modified at a time.

Suitable spacer groups that may be inserted between any two amino acid residues of the carrier moiety include alkyl groups such as methyl, ethyl or propyl groups in addition to amino acid spacers such as glycine or β-alanine residues. A further form of variation, type (e), involving the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example, Simon RJ et al., PNAS (1992) 89(20), 9367-9371 and Horwell DC, Trends Biotechnol. (1995) 13(4), 132-134. Type (f) modification may occur by methods such as those described in International Application PCT/GB99/01855 (WO 99/64574).

It is preferable for amino acid variation, preferably of type (a) or (b), to occur independently at any position. As mentioned above more than one homologous or non-homologous substitution may occur simultaneously. Further variation may occur by virtue of reversing the sequence of a number of amino acid residues within a sequence.

In one embodiment the replacement amino acid residue is a natural amino acid selected from the residues of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine.

The replacement amino acid residue may additionally be selected from unnatural amino acids. As used herein, the term “non-natural amino acid” or “unnatural amino acid” includes alpha and alpha-disubstituted amino acids, N-alkyl amino acids, lactic acid, halide derivatives of natural amino acids such as trifluorotyrosine, p-Cl-phenylalanine, p-F-phenylalanine, p-Br-phenylalanine, p-NO₂-phenylalanine, phenylglycine, sarcosine, penicillamine, D-2-methyltryptophan, phosphoserine, phosphothreonine, phosphotyrosine, p-I-phenylalanine, L-allyl-glycine, β-alanine, β-aspartic acid, β-cyclohexylalanine, citrulline, homoserine, homocysteine, pyroglutamic acid, L-α-amino butyric acid, L-γ-amino butyric acid, L-α-amino isobutyric acid, α-cyclohexylglycine, diaminobutyric acid, diaminopimelic acid, N-ε-dinitrophenyl-lysine, L-1-naphthylalanine, L-2-naphthylalanine, 3-(2-pyridyl)-L-alanine, 3-(3-pyridyl)-L-alanine, 3-(4-pyridyl)-L-alanine, N-ε-methyl-lysine, N,N-ε-dimethyl-lysine, N,N, N-ε-trimethyl-lysine, 3-mercaptopropionic acid, L-ε-amino caproic acid, 7-amino heptanoic acid, 6-amino hexanoic acid L-methionine sulfone, ornithine, L-norleucine, L-norvaline, p-nitro-L-phenylalanine, L-hydroxyproline, γ-glutamic acid, γ-amino butyric acid L-thioproline, methyl derivatives of phenylalanine (Phe) such as γ-methyl-Phe, pentamethyl-Phe, L-Phe (4-amino), L-Tyr (methyl), L-Phe (4-isopropyl), L-Tic (1,2,3,4tetrahydroiso-quinoline-3-carboxyl acid), L-diaminopropionic acid and L-Phe (4-benzyl).

In one preferred embodiment, the GLP peptide is a GLP-1 peptide, or an analogue or variant thereof.

In one preferred embodiment, the GLP-1 peptide is Liraglutaride, or an analogue or variant thereof.

Preferably, for this embodiment, the GLP-1 peptide or analogue or variant thereof is of SEQ ID NO: 1:

1 2 3 4 5 6 7 8 9 10 11 12
His-Ala-Glu-Gly-Thr-Phe-Thr-Ser-Asp-Val-Ser-Ser-
13 14 15 16 17 18 19 20 21 22
Tyr-Leu-Glu-Gly-Gln-Ala-Ala-Lys(Pal-Glu)-Glu-Phe-
23 24 25 26 27 28 29 30 31
Ile-Ala-Trp-Leu-Val-Arg-Gly-Arg-Gly-OH

and said process comprises:
(i) coupling a first peptide having the sequence:

[SEQ ID NO: 2]
His-Ala-Glu-Gly-Thr-Phe-Thr-Ser-Asp-Val-Ser-Ser-
Tyr-Leu-Glu-Gly-Gln-Ala-Ala

wherein:
the N-terminal of His is optionally protected with a protecting group, preferably Boc, or Fmoc; and
   the Ala carboxylic acid group is optionally in the form of an activated carboxylic acid derivative;
in solution with a second peptide having the sequence:

[SEQ ID NO: 3]
Lys(Pal-Glu-OX)-Glu-Phe-Ile-Ala-Trp-Leu-Val-Arg-
Gly-Arg-Gly-OH

wherein:
   X is H or a protecting group for the Glu carboxylic acid group;
and wherein one or more of the amino acid residues in said first and second peptides may be unprotected or protected, preferably with an acid-cleavable protecting group;
(ii) optionally removing any protecting groups;
(iii) optionally purifying the GLP-1 peptide.

In one preferred embodiment, the first peptide has the formula:

[SEQ ID NO: 4]
P1-His(P)-Ala-Glu(P)-Gly-Thr(P)-Phe-Thr(P)-Ser(P)-
Asp(P)-Val-Ser(P)-Ser(P)-Tyr(P)-Leu-Glu(P)-Gly-
Gln(P)-Ala-Ala-O-P2

wherein:
P1 is a protecting group for the N-terminal of His (preferably Boc, or Fmoc);
each P represents a side chain protecting group which may be the same or different; and
P2 is H or an activated carboxylic ester of the Ala residue (preferably Su, Bt or Pfp).

In a more preferred embodiment, the first peptide is selected from:

[SEQ ID NO: 5]
Boc-His(Trt)-Ala-Glu(tBu)-Gly-Thr(tBu)-Phe-
Thr(tBu)-Ser(tBu)-Asp(tBu)-Val-Ser(tBu)-
Ser(tBu)-Tyr(tBu)-Leu-Glu(tBu)-Gly-Gln(Trt)-
Ala-Ala-OH;
[SEQ ID NO: 59]
Boc-His(Trt)-Ala-Glu(tBu)-Gly-Thr(tBu)-Phe-
Thr(tBu)-Ser(tBu)-Asp(tBu)Val-Ser(tBu)-
ψSer-Tyr(tBu)-Leu-Glu(tBu)-Gly-Gln(Trt)-Ala-
Ala-OH,
(where ψSer is a pseudoproline);
and
[SEQ ID NO: 62]
Boc-His(Trt)-Ala-Glu(tBu)-Gly-Thr(tBu)-Phe-
ψThr-Ser(tBu)-Asp(tBu)-Val-Ser(tBu)-
ψSer-Tyr(tBu)-Leu-Glu(tBu)-Gly-Gln(Trt)-Ala-
Ala-OH,
(where ψSer and ψThr are pseudoprolines).

In one preferred embodiment, the second peptide has the formula:

[SEQ ID NO: 6]
Lys(Pal-Glu)-Glu(P)-Phe-Ile-Ala-Trp(P)-Leu-Val-
Arg(P)-Gly-Arg(P)-Gly-O-P3

wherein each P represents a side chain protecting group which may be the same or different; and
P3 is H or a carboxy protecting group, preferably selected from Clt, Trt, tBu, DPM, MeDPM and MeODPM.

In a more preferred embodiment, the second peptide is H-Lys(Pal-Glu)-Glu(tBu)-Phe-Ile-Ala-Trp(Boc)-Leu-Val-Arg(Pbf)-Gly-Arg(Pbf)-Gly-O-Clt [SEQ ID NO: 7] or H-Lys(Pal-Glu)-Glu(tBu)-Phe-Ile-Ala-Trp(Boc)-Leu-Val-Arg(Pbf)-Gly-Arg(Pbf)-Gly-O-tBu [SEQ ID NO: 63].

In one highly preferred embodiment for preparing Liraglutide, the first peptide is [SEQ ID NO: 62] and the second peptide is [SEQ ID NO: 63].

In another preferred embodiment, the GLP-1 peptide or analogue or variant thereof is semaglutide or a variant thereof.

Preferably, for this embodiment, the GLP-1 peptide is of SEQ ID NO: 8:

1 2 3 4 5 6 7 8 9 10 11
His-Aib-Glu-Gly-Thr-Phe-Thr-Ser-Asp-Val-Ser-
12 13 14 15 16 17 18 19 20
Ser-Tyr-Leu-Glu-Gly-Gln-Ala-Ala-Lys(W)-Glu-
21 22 23 24 25 26 27 28 29 30 31
Phe-Ile-Ala-Trp-Leu-Val-Arg-Gly-Arg-Gly-OH

where W is N-(17-carboxy-1-oxoheptadecyl)-L-γ-glutamyl-2-[2-(2-aminoethoxy) ethoxy]acetyl-2-[2-(2-aminoethoxy)ethoxy]acetyl;
and said process comprises:
(i) coupling a first peptide having the sequence:

[SEQ ID NO: 9]
His-Aib-Glu-Gly-Thr-Phe-Thr-Ser-Asp-Val-Ser-
Ser-Tyr-Leu-Glu-Gly-Gln-Ala-Ala

wherein:
the N-terminal of His is optionally protected with a protecting group, preferably Boc, or Fmoc; and
   the Ala carboxylic acid group is optionally in the form of an activated carboxylic acid derivative;
in solution with a second peptide having the sequence:

[SEQ ID NO: 10]
Lys(W1)-Glu-Phe-Ile-Ala-Trp-Leu-Val-Arg-Gly-
Arg-Gly-OH

wherein:
   W1 is N-(17-carboxy-1-oxoheptadecyl)-L-γ-glutamyl-2-[2-(2-aminoethoxy) ethoxy]acetyl-2-[2-(2-aminoethoxy)ethoxy]acetyl;
and wherein one or more of the amino acid residues in said first and second peptides and W1 may be unprotected or protected, preferably with an acid-cleavable protecting group;
(ii) optionally removing any protecting groups;
(iii) optionally purifying the GLP-1 peptide.

In one preferred embodiment, the first peptide has the formula:

[SEQ ID NO: 11]
P1-His(P)-Aib-Glu(P)-Gly-Thr(P)-Phe-Thr(P)-
Ser(P)-Asp(P)-Val-Ser(P)-Ser(P)-Tyr(P)-Leu-
Glu(P)-Gly-Gln(P)-Ala-Ala-O-P2

wherein:
P1 is a protecting group for the N-terminal of His (preferably Boc, or Fmoc);

each P represents a side chain protecting group which may be the same or different; and
P2 is H or an activated carboxylic ester of the Ala residue (preferably Su, Bt or Pfp).

In a more preferred embodiment, the first peptide is selected from:

[SEQ ID NO: 12]
Boc-His(Trt)-Aib-Glu(tBu)-Gly-Thr(tBu)-Phe-
Thr(tBu)-Ser(tBu)-Asp(tBu)-Val-Ser(tBu)-
Ser(tBu)-Tyr(tBu)-Leu-Glu(tBu)-Gly-Gln(Trt)-
Ala-Ala-OH;
[SEQ ID NO: 60]
Boc-His(Trt)-Aib-Glu(tBu)-Gly-Thr(tBu)-Phe-
Thr(tBu)-Ser(tBu)-Asp(tBu)-Val-Ser(tBu)-
ψSer-Tyr(tBu)-Leu-Glu(tBu)-Gly-Gln(Trt)-Ala-
Ala-OH,
(where ψSer is a pseudoproline);
and
[SEQ ID NO: 61]
Boc-His(Trt)-Aib-Glu(tBu)-Gly-Thr(tBu)-Phe-
ψThr-Ser(tBu)-Asp(tBu)-Val-Ser(tBu)-
ψSer-Tyr(tBu)-Leu-Glu(tBu)-Gly-Gln(Trt)-Ala-
Ala-OH
(where ψSer and ψThr are pseudoprolines).

In one preferred embodiment, the second peptide has the formula:

[SEQ ID NO: 13]
Lys(W)-Glu(P)-Phe-Ile-Ala-Trp(P)-Leu-Val-
Arg(P)-Gly-Arg(P)-Gly-O-P3

wherein:
W is N-(17-carboxy-1-oxoheptadecyl)-L-γ-glutamyl-2-[2-(2-aminoethoxy) ethoxy]acetyl-2-[2-(2-aminoethoxy)ethoxy]acetyl;
each P represents a side chain protecting group which may be the same or different; and
P3 is a carboxy protecting group, preferably selected from Clt, Trt, tBu, DPM, MeDPM and MeODPM.

In a more preferred embodiment, the second peptide is H-Lys(W)-Glu(tBu)-Phe-Ile-Ala-Trp(Boc)-Leu-Val-Arg(Pbf)-Gly-Arg(Pbf)-Gly-O-Clt, where W is N-(17-carboxy-1-oxoheptadecyl)-L-γ-glutamyl-2-[2-(2-aminoethoxy) ethoxy]acetyl-2-[2-(2-aminoethoxy)ethoxy]acetyl [SEQ ID NO: 14], or H-Lys(Pal-Glu)-Glu(tBu)-Phe-Ile-Ala-Trp(Boc)-Leu-Val-Arg(Pbf)-Gly-Arg(Pbf)-Gly-O-tBu, where W is N-(17-carboxy-1-oxoheptadecyl)-L-γ-glutamyl-2-[2-(2-aminoethoxy) ethoxy]acetyl-2-[2-(2-aminoethoxy)ethoxy]acetyl [SEQ ID NO: 64].

In one highly preferred embodiment for preparing Semaglutide, the first peptide is [SEQ ID NO: 61] and the second peptide is [SEQ ID NO: 64].

In another embodiment, the GLP-1 peptide or analogue or variant thereof is of SEQ ID NO: 1:

1 2 3 4 5 6 7 8 9 10 11
His-Ala-Glu-Gly-Thr-Phe-Thr-Ser-Asp-Val-Ser-
12 13 14 15 16 17 18 19 20
Ser-Tyr-Leu-Glu-Gly-Gln-Ala-Ala-Lys(Pal-Glu)-
21 22 23 24 25 26 27 28 29 30 31
Glu-Phe-Ile-Ala-Trp-Leu-Val-Arg-Gly-Arg-Gly-OH

and said process comprises:
(i) coupling a first peptide having the sequence:

[SEQ ID NO: 15]
His-Ala-Glu-Gly-Thr-Phe-Thr-Ser-Asp-Val-Ser-
Ser-Tyr-Leu-Glu-Gly-Gln-Ala

wherein:
the N-terminal of His is optionally protected with a protecting group, preferably Boc, or Fmoc; and
   the Ala carboxylic acid group is optionally in the form of an activated carboxylic acid derivative;
in solution with a second peptide having the sequence:

[SEQ ID NO: 16]
Ala-Lys(Pal-Glu-OX)-Glu-Phe-Ile-Ala-Trp-Leu-
Val-Arg-Gly-Arg-Gly-OH

wherein:
   X is H or a protecting group for the Glu carboxylic acid group;
and wherein one or more of the amino acid residues in said first and second peptides may be unprotected or protected, preferably with an acid-cleavable protecting group;
(ii) optionally removing any protecting groups;
(iii) optionally purifying the GLP-1 peptide.

In one preferred embodiment, the first peptide has the formula:

[SEQ ID NO: 17]
P1-His(P)-Ala-Glu(P)-Gly-Thr(P)-Phe-Thr(P)-
Ser(P)-Asp(P)-Val-Ser(P)-Ser(P)-Tyr(P)-Leu-
Glu(P)-Gly-Gln(P)-Ala-O-P2

wherein:
P1 is a protecting group for the N-terminal of His (preferably Boc, or Fmoc);
each P represents a side chain protecting group which may be the same or different; and
P2 is H or an activated carboxylic ester of the Ala residue (preferably Su, Bt or Pfp).

In a more preferred embodiment, the first peptide is selected from:

[SEQ ID NO: 18]
Boc-His(Trt)-Ala-Glu(tBu)-Gly-Thr(tBu)-Phe-
Thr(tBu)-Ser(tBu)-Asp(tBu)-Val-Ser(tBu)-
Ser(tBu)-Tyr(tBu)-Leu-Glu(tBu)-Gly-Gln(Trt)-
Ala-OH;
[SEQ ID NO: 65]
Boc-His(Trt)-Ala-Glu(tBu)-Gly-Thr(tBu)-Phe-
Thr(tBu)-Ser(tBu)-Asp(tBu)-Val-Ser(tBu)-
ψSer-Tyr(tBu)-Leu-Glu(tBu)-Gly-Gln(Trt)-Ala-
OH;
and
[SEQ ID NO: 66]
Boc-His(Trt)-Ala-Glu(tBu)-Gly-Thr(tBu)-Phe-
ψThr-Ser(tBu)-Asp(tBu)-Val-Ser(tBu)-
ψSer-Tyr(tBu)-Leu-Glu(tBu)-Gly-Gln(Trt)-Ala-
OH.

In one preferred embodiment, the second peptide has the formula:

[SEQ ID NO: 19]
Ala-Lys(Pal-Glu)-Glu(P)-Phe-Ile-Ala-Trp(P)-
Leu-Val-Arg(P)-Gly-Arg(P)-Gly-O-P3

wherein each P represents a side chain protecting group which may be the same or different; and
P3 is a carboxy protecting group, preferably selected from Clt, Trt, tBu, DPM, MeDPM and MeODPM.

In a more preferred embodiment, the second peptide is H-Ala-Lys(Pal-Glu)-Glu(tBu)-Phe-Ile-Ala-Trp(Boc)-Leu-Val-Arg(Pbf)-Gly-Arg(Pbf)-Gly-O-Clt [SEQ ID NO: 20] or H-Ala-Lys(Pal-Glu)-Glu(tBu)-Phe-Ile-Ala-Trp(Boc)-Leu-Val-Arg(Pbf)-Gly-Arg(Pbf)-Gly-O-tBu [SEQ ID NO: 67].

In another embodiment, the GLP-1 peptide or analogue or variant thereof is of SEQ ID NO: 8:

1 2 3 4 5 6 7 8 9 10 11
His-Aib-Glu-Gly-Thr-Phe-Thr-Ser-Asp-Val-Ser-
12 13 14 15 16 17 18 19 20
Ser-Tyr-Leu-Glu-Gly-Gln-Ala-Ala-Lys(W)-Glu-
21 22 23 24 25 26 27 28 29 30 31
Phe-Ile-Ala-Trp-Leu-Val-Arg-Gly-Arg-Gly-OH

where W is N-(17-carboxy-1-oxoheptadecyl)-L-γ-glutamyl-2-[2-(2-aminoethoxy) ethoxy]acetyl-2-[2-(2-aminoethoxy)ethoxy]acetyl;
and said process comprises:
(i) coupling a first peptide having the sequence:

[SEQ ID NO: 21]
His-Aib-Glu-Gly-Thr-Phe-Thr-Ser-Asp-Val-Ser-
Ser-Tyr-Leu-Glu-Gly-Gln-Ala

wherein:
the N-terminal of His is optionally protected with a protecting group, preferably Boc, or Fmoc; and
   the Ala carboxylic acid group is optionally in the form of an activated carboxylic acid derivative;
in solution with a second peptide having the sequence:

[SEQ ID NO: 22]
Ala-Lys(W1)-Glu-Phe-Ile-Ala-Trp-Leu-Val-Arg-Gly-
Arg-Gly-OH

wherein:
   W1 is N-(17-carboxy-1-oxoheptadecyl)-L-γ-glutamyl-2-[2-(2-aminoethoxy) ethoxy]acetyl-2-[2-(2-aminoethoxy)ethoxy]acetyl;
and wherein one or more of the amino acid residues in said first and second peptides and W1 may be unprotected or protected, preferably with an acid-cleavable protecting group;
(ii) optionally removing any protecting groups;
(iii) optionally purifying the GLP-1 peptide.

In one preferred embodiment, the first peptide has the formula:

[SEQ ID NO: 23]
P1-His(P)-Aib-Glu(P)-Gly-Thr(P)-Phe-Thr(P)-
Ser(P)-Asp(P)-Val-Ser(P)-Ser(P)-Tyr(P)-Leu-
Glu(P)-Gly-Gln(P)-Ala-O-P2

wherein:
P1 is a protecting group for the N-terminal of His (preferably Boc, or Fmoc);
each P represents a side chain protecting group which may be the same or different; and
P2 is H or an activated carboxylic ester of the Ala residue (preferably Su, Bt or Pfp).

In a more preferred embodiment, the first peptide is selected from:

[SEQ ID NO: 24]
Boc-His(Trt)-Aib-Glu(tBu)-Gly-Thr(tBu)-
Phe-Thr(tBu)-Ser(tBu)-Asp(tBu)-Val-Ser(tBu)-
Ser(tBu)-Tyr(tBu)-Leu-Glu(tBu)-Gly-Gln(Trt)-Ala-
Ala-OH;
[SEQ ID NO: 68]
Boc-His(Trt)-Aib-Glu(tBu)-Gly-Thr(tBu)-Phe-
Thr(tBu)-Ser(tBu)-Asp(tBu)-Val-Ser(tBu)-ΨSer-
Tyr(tBu)-Leu-Glu(tBu)-Gly-Gln(Trt)-Ala-Ala-OH;
and
[SEQ ID NO: 69]
Boc-His(Trt)-Aib-Glu(tBu)-Gly-Thr(tBu)-Phe-ΨThr-
Ser(tBu)-Asp(tBu)-Val-Ser(tBu)-ΨSer-Tyr(tBu)-
Leu-Glu(tBu)-Gly-Gln(Trt)-Ala-Ala-OH.

In one preferred embodiment, the second peptide has the formula:

[SEQ ID NO: 25]
Ala-Lys(W)-Glu(P)-Phe-Ile-Ala-Trp(P)-Leu-Val-
Arg(P)-Gly-Arg(P)-Gly-O-P3

wherein:
W is N-(17-carboxy-1-oxoheptadecyl)-L-γ-glutamyl-2-[2-(2-aminoethoxy) ethoxy]acetyl-2-[2-(2-aminoethoxy)ethoxy]acetyl;
each P represents a side chain protecting group which may be the same or different; and
P3 is a carboxy protecting group, preferably selected from Clt, Trt, tBu, DPM, MeDPM and MeODPM.

In a more preferred embodiment, the second peptide is H-Ala-Lys(W)-Glu(tBu)-Phe-Ile-Ala-Trp(Boc)-Leu-Val-Arg(Pbf)-Gly-Arg(Pbf)-Gly-O-Clt, where W is N-(17-carboxy-1-oxoheptadecyl)-L-γ-glutamyl-2-[2-(2-aminoethoxy) ethoxy]acetyl-2-[2-(2-aminoethoxy)ethoxy]acetyl [SEQ ID NO: 26] or H-Ala-Lys(W)-Glu(tBu)-Phe-Ile-Ala-Trp(Boc)-Leu-Val-Arg(Pbf)-Gly-Arg(Pbf)-Gly-O-tBu, where W is N-(17-carboxy-1-oxoheptadecyl)-L-γ-glutamyl-2-[2-(2-aminoethoxy) ethoxy] acetyl-2-[2-(2-aminoethoxy)ethoxy]acetyl [SEQ ID NO: 70].

In another embodiment, the GLP-1 peptide or analogue or variant thereof is of SEQ ID NO: 1:

1 2 3 4 5 6 7 8 9 10 11 12
His-Ala-Glu-Gly-Thr-Phe-Thr-Ser-Asp-Val-Ser-Ser-
13 14 15 16 17 18 19 20 21 22
Tyr-Leu-Glu-Gly-Gln-Ala-Ala-Lys(Pal-Glu)-Glu-Phe-
23 24 25 26 27 28 29 30 31
Ile-Ala-Trp-Leu-Val-Arg-Gly-Arg-Gly-OH

and said process comprises:
(i) coupling a first peptide having the sequence:

[SEQ ID NO: 27]
His-Ala-Glu-Gly-Thr-Phe-Thr-Ser-Asp-Val-Ser-Ser-
Tyr-Leu-Glu-Gly-Gln

wherein:
the N-terminal of His is optionally protected with a protecting group, preferably Boc, or Fmoc; and
   the Gln carboxylic acid group is optionally in the form of an activated carboxylic acid derivative;
in solution with a second peptide having the sequence:

[SEQ ID NO: 28]
Ala-Ala-Lys(Pal-Glu-OX)-Glu-Phe-Ile-Ala-Trp-Leu-
Val-Arg-Gly-Arg-Gly-OH

wherein:
   X is H or a protecting group for the Glu carboxylic acid group;
and wherein one or more of the amino acid residues in said first and second peptides may be unprotected or protected, preferably with an acid-cleavable protecting group;
(ii) optionally removing any protecting groups;
(iii) optionally purifying the GLP-1 peptide.

In one preferred embodiment, the first peptide has the formula:

[SEQ ID NO: 29]
P1-His(P)-Ala-Glu(P)-Gly-Thr(P)-Phe-Thr(P)-
Ser(P)-Asp(P)-Val-Ser(P)-Ser(P)-Tyr(P)-Leu-
Glu(P)-Gly-Gln-O-P2

wherein:
P1 is a protecting group for the N-terminal of His (preferably Boc, or Fmoc);
each P represents a side chain protecting group which may be the same or different; and
P2 is H or an activated carboxylic ester of the Gln residue (preferably Su, Bt or Pfp).

In a more preferred embodiment, the first peptide is selected from:

[SEQ ID NO: 30]
Boc-His(Trt)-Ala-Glu(tBu)-Gly-Thr(tBu)-Phe-
Thr(tBu)-Ser(tBu)-Asp(tBu)-Val-Ser(tBu)-
Ser(tBu)-Tyr(tBu)-Leu-Glu(tBu)-Gly-Gln-OH;
[SEQ ID NO: 71]
Boc-His(Trt)-Ala-Glu(tBu)-Gly-Thr(tBu)-Phe-
Thr(tBu)-Ser(tBu)-Asp(tBu)-Val-Ser(tBu)-Ψ Ser-
Tyr(tBu)-Leu-Glu(tBu)-Gly-Gln-OH;
and
[SEQ ID NO: 72]
Boc-His(Trt)-Ala-Glu(tBu)-Gly-Thr(tBu)-Phe-ΨThr-
Ser(tBu)-Asp(tBu)-Val-Ser(tBu)- ΨSer-Tyr(tBu)-
Leu-Glu(tBu)-Gly-Gln-OH.

In one preferred embodiment, the second peptide has the formula:

[SEQ ID NO: 31]
Ala-Ala-Lys(Pal-Glu)-Glu(P)-Phe-Ile-Ala-Trp(P)-
Leu-Val-Arg(P)-Gly-Arg(P)-Gly-O-P3

wherein each P represents a side chain protecting group which may be the same or different; and
P3 is a carboxy protecting group, preferably selected from Clt, Trt, tBu, DPM, MeDPM and MeODPM.

In a more preferred embodiment, the second peptide is H-Ala-Ala-Lys(Pal-Glu)-Glu(tBu)-Phe-Ile-Ala-Trp(Boc)-Leu-Val-Arg(Pbf)-Gly-Arg(Pbf)-Gly-O-Clt [SEQ ID NO: 32] or H-Ala-Ala-Lys(Pal-Glu)-Glu(tBu)-Phe-Ile-Ala-Trp(Boc)-Leu-Val-Arg(Pbf)-Gly-Arg(Pbf)-Gly-O-tBu [SEQ ID NO: 73].

In one preferred embodiment, the GLP-1 peptide or analogue or variant thereof is of

1 2 3 4 5 6 7 8 9 10 11 12
His-Aib-Glu-Gly-Thr-Phe-Thr-Ser-Asp-Val-Ser-Ser-
13 14 15 16 17 18 19 20 21 22 23
Tyr-Leu-Glu-Gly-Gln-Ala-Ala-Lys(W)-Glu-Phe-Ile-
24 25 26 27 28 29 30 31
Ala-Trp-Leu-Val-Arg-Gly-Arg-Gly-OH

where W is N-(17-carboxy-1-oxoheptadecyl)-L-γ-glutamyl-2-[2-(2-aminoethoxy) ethoxy]acetyl-2-[2-(2-aminoethoxy)ethoxy]acetyl;
and said process comprises:
(i) coupling a first peptide having the sequence:

[SEQ ID NO: 33]
His-Aib-Glu-Gly-Thr-Phe-Thr-Ser-Asp-Val-Ser-Ser-
Tyr-Leu-Glu-Gly-Gln

wherein:
the N-terminal of His is optionally protected with a protecting group, preferably Boc, or Fmoc; and
   the Gln carboxylic acid group is optionally in the form of an activated carboxylic acid derivative;
in solution with a second peptide having the sequence:

[SEQ ID NO: 34]
Ala-Ala-Lys(W1)-Glu-Phe-Ile-Ala-Trp-Leu-Val-Arg-
Gly-Arg-Gly-OH

wherein:
   W1 is N-(17-carboxy-1-oxoheptadecyl)-L-γ-glutamyl-2-[2-(2-aminoethoxy) ethoxy]acetyl-2-[2-(2-aminoethoxy)ethoxy]acetyl;
   and wherein one or more of the amino acid residues in said first and second peptides and W1 may be unprotected or protected, preferably with an acid-cleavable protecting group;
(ii) optionally removing any protecting groups;
(iii) optionally purifying the GLP-1 peptide.

In one preferred embodiment, the first peptide has the formula:

[SEQ ID NO: 35]
P1-His(P)-Aib-Glu(P)-Gly-Thr(P)-Phe-Thr(P)-
Ser(P)-Asp(P)-Val-Ser(P)-Ser(P)-Tyr(P)-Leu-
Glu(P)-Gly-Gln-O-P2

wherein:
P1 is a protecting group for the N-terminal of His (preferably Boc, or Fmoc);
each P represents a side chain protecting group which may be the same or different; and
P2 is H or an activated carboxylic ester of the Gln residue (preferably Su, Bt or Pfp).

In a more preferred embodiment, the first peptide is selected from:

[SEQ ID NO: 36]
Boc-His(Trt)-Aib-Glu(tBu)-Gly-Thr(tBu)-Phe-
Thr(tBu)-Ser(tBu)-Asp(tBu)-Val-Ser(tBu)-
Ser(tBu)-Tyr(tBu)-Leu-Glu(tBu)-Gly-Gln-OH;
[SEQ ID NO: 74]
Boc-His(Trt)-Aib-Glu(tBu)-Gly-Thr(tBu)-Phe-
Thr(tBu)-Ser(tBu)-Asp(tBu)-Val-Ser(tBu)-ΨSer-
Tyr(tBu)-Leu-Glu(tBu)-Gly-Gln-OH;
and
[SEQ ID NO: 75]
Boc-His(Trt)-Aib-Glu(tBu)-Gly-Thr(tBu)-Phe-
ΨThr-Ser(tBu)-Asp(tBu)-Val-Ser(tBu)-ΨSer-Tyr(tBu)-
Leu-Glu(tBu)-Gly-Gln-OH.

In one preferred embodiment, the second peptide has the formula:

[SEQ ID NO: 37]
Ala-Ala-Lys(W)-Glu(P)-Phe-Ile-Ala-Trp(P)-
Leu-Val-Arg(P)-Gly-Arg(P)-Gly-O-P3

wherein:
W is N-(17-carboxy-1-oxoheptadecyl)-L-γ-glutamyl-2-[2-(2-aminoethoxy) ethoxy]acetyl-2-[2-(2-aminoethoxy)ethoxy]acetyl;
each P represents a side chain protecting group which may be the same or different; and
P3 is a carboxy protecting group, preferably selected from Clt, Trt, tBu, DPM, MeDPM and MeODPM.

In a more preferred embodiment, the second peptide is H-Ala-Ala-Lys(W)-Glu(tBu)-Phe-Ile-Ala-Trp(Boc)-Leu-Val-Arg(Pbf)-Gly-Arg(Pbf)-Gly-O-Clt, where W is N-(17-carboxy-1-oxoheptadecyl)-L-γ-glutamyl-2-[2-(2-aminoethoxy) ethoxy]acetyl-2-[2-(2-aminoethoxy)ethoxy]acetyl [SEQ ID NO: 38], or H-Ala-Ala-Lys(W)-Glu(tBu)-Phe-Ile-Ala-Trp(Boc)-Leu-Val-Arg(Pbf)-Gly-Arg(Pbf)-Gly-O-tBu, where W is N-(17-carboxy-1-oxoheptadecyl)-L-γ-glutamyl-2-[2-(2-aminoethoxy) ethoxy]acetyl-2-[2-(2-aminoethoxy)ethoxy]acetyl [SEQ ID NO: 76].

In another embodiment, the GLP-1 peptide or analogue or variant thereof is of SEQ ID NO: 1:

1 2 3 4 5 6 7 8 9 10
His-Ala-Glu-Gly-Thr-Phe-Thr-Ser-Asp-Val-
11 12 13 14 15 16 17 18 19
Ser-Ser-Tyr-Leu-Glu-Gly-Gln-Ala-Ala-
20 21 22 23 24 25 26
Lys(Pal-Glu)-Glu-Phe-Ile-Ala-Trp-Leu-
27 28 29 30 31
Val-Arg-Gly-Arg-Gly-OH

and said process comprises:
(i) coupling a first peptide having the sequence:

[SEQ ID NO: 39]
His-Ala-Glu-Gly-Thr-Phe-Thr-Ser-Asp-Val-Ser-Ser-
Tyr-Leu

wherein:
the N-terminal of His is optionally protected with a protecting group, preferably Boc, or Fmoc; and
   the Leu carboxylic acid group is optionally in the form of an activated carboxylic acid derivative;
in solution with a second peptide having the sequence:

[SEQ ID NO: 40]
Glu-Gly-Gln-Ala-Ala-Lys(Pal-Glu-OX)-Glu-Phe-
Ile-Ala-Trp-Leu-Val-Arg-Gly-Arg-Gly-OH

wherein:
   X is H or a protecting group for the Glu carboxylic acid group;
and wherein one or more of the amino acid residues in said first and second peptides may be unprotected or protected, preferably with an acid-cleavable protecting group;
(ii) optionally removing any protecting groups;
(iii) optionally purifying the GLP-1 peptide.

In one preferred embodiment, the first peptide has the formula:

[SEQ ID NO: 41]
P1-His(P)-Ala-Glu(P)-Gly-Thr(P)-Phe-Thr(P)-
Ser(P)-Asp(P)-Val-Ser(P)-Ser(P)-Tyr(P)-
Leu-O-P2

wherein:
P1 is a protecting group for the N-terminal of His (preferably Boc, or Fmoc);
each P represents a side chain protecting group which may be the same or different; and
P2 is H or an activated carboxylic ester of the Leu residue (preferably Su, Bt or Pfp).

In a more preferred embodiment, the first peptide is selected from:

[SEQ ID NO: 42]
Boc-His(Trt)-Ala-Glu(tBu)-Gly-Thr(tBu)-Phe-
Thr(tBu)-Ser(tBu)-Asp(tBu)-Val-Ser(tBu)-
Ser(tBu)-Tyr(tBu)-Leu-OH;
[SEQ ID NO: 77]
Boc-His(Trt)-Ala-Glu(tBu)-Gly-Thr(tBu)-Phe-
Thr(tBu)-Ser(tBu)-Asp(tBu)-Val-Ser(tBu)-
ΨSer-Tyr(tBu)-Leu-OH;
and
[SEQ ID NO: 78]
Boc-His(Trt)-Ala-Glu(tBu)-Gly-Thr(tBu)-Phe-
ΨThr-Ser(tBu)-Asp(tBu)-Val-Ser(tBu)-
ΨSer-Tyr(tBu)-Leu-OH.

In one preferred embodiment, the second peptide has the formula:

[SEQ ID NO: 43]
Glu(P)-Gly-Gln(P)-Ala-Ala-Lys(Pal-Glu)-
Glu(P)-Phe-Ile-Ala-Trp(P)-Leu-Val-
Arg(P)-Gly-Arg(P)-Gly-O-P3

wherein each P represents a side chain protecting group which may be the same or different; and
P3 is a carboxy protecting group, preferably selected from Clt, Trt, tBu, DPM, MeDPM and MeODPM.

In a more preferred embodiment, the second peptide is H-Glu(tBu)-Gly-Gln(Trt)-Ala-Ala-Ala-Lys(Pal-Glu)-Glu(tBu)-Phe-Ile-Ala-Trp(Boc)-Leu-Val-Arg(Pbf)-Gly-Arg(Pbf)-Gly-O-Clt [SEQ ID NO: 44], or H-Glu(tBu)-Gly-Gln(Trt)-Ala-Ala-Ala-Lys(Pal-Glu)-Glu(tBu)-Phe-Ile-Ala-Trp(Boc)-Leu-Val-Arg(Pbf)-Gly-Arg(Pbf)-Gly-O-tBu [SEQ ID NO: 79].

In one preferred embodiment, the GLP-1 peptide or analogue or variant thereof is of SEQ ID NO: 8:

1 2 3 4 5 6 7 8 9 10
His-Aib-Glu-Gly-Thr-Phe-Thr-Ser-Asp-Val-
11 12 13 14 15 16 17 18 19
Ser-Ser-Tyr-Leu-Glu-Gly-Gln-Ala-Ala-
20 21 22 23 24 25 26 27 28
Lys(W)-Glu-Phe-Ile-Ala-Trp-Leu-Val-Arg-
29 30 31
Gly-Arg-Gly-OH

where W is N-(17-carboxy-1-oxoheptadecyl)-L-γ-glutamyl-2-[2-(2-aminoethoxy) ethoxy]acetyl-2-[2-(2-aminoethoxy)ethoxy]acetyl;
and said process comprises:
(i) coupling a first peptide having the sequence:

[SEQ ID NO: 45]
His-Aib-Glu-Gly-Thr-Phe-Thr-Ser-Asp-Val-
Ser-Ser-Tyr-Leu

wherein:
the N-terminal of His is optionally protected with a protecting group, preferably Boc, or Fmoc; and
   the Leu carboxylic acid group is optionally in the form of an activated carboxylic acid derivative;
in solution with a second peptide having the sequence:

[SEQ ID NO: 46]
Glu-Gly-Gln-Ala-Ala-Lys(W1)-Glu-Phe-Ile-
Ala-Trp-Leu-Val-Arg-Gly-Arg-Gly-OH

wherein:
   W1 is N-(17-carboxy-1-oxoheptadecyl)-L-γ-glutamyl-2-[2-(2-aminoethoxy) ethoxy]acetyl-2-[2-(2-aminoethoxy)ethoxy]acetyl;
and wherein one or more of the amino acid residues in said first and second peptides and W1 may be unprotected or protected, preferably with an acid-cleavable protecting group;
(ii) optionally removing any protecting groups;
(iii) optionally purifying the GLP-1 peptide.

In one preferred embodiment, the first peptide has the formula:

[SEQ ID NO: 47]
P1-His(P)-Aib-Glu(P)-Gly-Thr(P)-Phe-Thr(P)-
Ser(P)-Asp(P)-Val-Ser(P)-Ser(P)-Tyr(P)-
Leu-O-P2

wherein:
P1 is a protecting group for the N-terminal of His (preferably Boc, or Fmoc);
each P represents a side chain protecting group which may be the same or different; and
P2 is H or an activated carboxylic ester of the Leu residue (preferably Su, Bt or Pfp).

In a more preferred embodiment, the first peptide is selected from:

[SEQ ID NO: 48]
Boc-His(Trt)-Aib-Glu(tBu)-Gly-Thr(tBu)-Phe-
Thr(tBu)-Ser(tBu)-Asp(tBu)-Val-Ser(tBu)-
Ser(tBu)-Tyr(tBu)-Leu-OH;
[SEQ ID NO: 80]
Boc-His(Trt)-Aib-Glu(tBu)-Gly-Thr(tBu)-Phe-
Thr(tBu)-Ser(tBu)-Asp(tBu)-Val-Ser(tBu)-
ΨSer-Tyr(tBu)-Leu-OH;
and
[SEQ ID NO: 81]
Boc-His(Trt)-Aib-Glu(tBu)-Gly-Thr(tBu)-Phe-
ΨThr-Ser(tBu)-Asp(tBu)-Val-Ser(tBu)-
ΨSer-Tyr(tBu)-Leu-OH.

In one preferred embodiment, the second peptide has the formula:

[SEQ ID NO: 49]
Glu(P)-Gly-Gln(P)-Ala-Ala-Lys(W)-Glu(P)-
Phle-Ile-Ala-Trp(P)-Leu-Val-Arg(P)-Gly-
Arg(P)-Gly-O-P3

wherein:
W is N-(17-carboxy-1-oxoheptadecyl)-L-γ-glutamyl-2-[2-(2-aminoethoxy) ethoxy]acetyl-2-[2-(2-aminoethoxy)ethoxy]acetyl;
each P represents a side chain protecting group which may be the same or different; and
P3 is a carboxy protecting group, preferably selected from Clt, Trt, tBu, DPM, MeDPM and MeODPM.

In a more preferred embodiment, the second peptide is H-Glu(tBu)-Gly-Gln(Trt)-Ala-Lys(W)-Glu(tBu)-Phe-Ile-Ala-Trp(Boc)-Leu-Val-Arg(Pbf)-Gly-Arg(Pbf)-Gly-O-Clt, where W is N-(17-carboxy-1-oxoheptadecyl)-L-γ-glutamyl-2-[2-(2-aminoethoxy) ethoxy]acetyl-2-[2-(2-aminoethoxy)ethoxy]acetyl [SEQ ID NO: 50] or or H-Glu(tBu)-Gly-Gln(Trt)-Ala-Lys(W)-Glu(tBu)-Phe-Ile-Ala-Trp(Boc)-Leu-Val-Arg(Pbf)-Gly-Arg(Pbf)-Gly-O-tBu, where W is N-(17-carboxy-1-oxoheptadecyl)-L-γ-glutamyl-2-[2-(2-aminoethoxy) ethoxy]acetyl-2-[2-(2-aminoethoxy)ethoxy]acetyl [SEQ ID NO: 82].

In one preferred embodiment the GLP peptide is a GLP-2 peptide or an analogue or variant thereof.

In one highly preferred embodiment the GLP-2 peptide or analogue or variant thereof is Teduglutide, or an analogue or variant thereof.

In one preferred embodiment, the GLP-2 peptide is a variant of Teduglutide in which the Gly residue in the 2-position is substituted with Aib (“Aib2-Ted”), i.e. SEQ ID NO: 108:

1 2 3 4 5 6 7 8 9 10
His-Aib-Asp-Gly-Ser-Phe-Ser-Asp-Glu-Met-
11 12 13 14 15 16 17 18 19 20
Asn-Thr-Ile-Leu-Asp-Asn-Leu-Ala-Ala-Arg-
21 22 23 24 25 26 27 28 29 30
Asp-Phe-Ile-Asn-Trp-Leu-Ile-Gln-Thr-Lys-
31 32 33
Ile-Thr-Asp

In one preferred embodiment the GLP-2 peptide or analogue or variant thereof is of SEQ ID NO: 83:

1 2 3 4 5 6 7 8 9 10
His-Gly-Asp-Gly-Ser-Phe-Ser-Asp-Glu-Met-
11 12 13 14 15 16 17 18 19 20
Asn-Thr-Ile-Leu-Asp-Asn-Leu-Ala-Ala-Arg-
21 22 23 24 25 26 27 28 29 30
Asp-Phe-Ile-Asn-Trp-Leu-Ile-Gln-Thr-Lys-
31 32 33
Ile-Thr-Asp

or a variant thereof,
and said process comprises:
(i) coupling a first peptide having the sequence:

[SEQ ID NO: 84]
His-Gly-Asp-Gly-Ser-Phe-Ser-Asp-Glu-
Met-Asn-Thr-Ile-Leu

wherein:
the N-terminal of His is optionally protected with a protecting group, preferably Boc, or Fmoc; and
   the Leu carboxylic acid group is optionally in the form of an activated carboxylic acid derivative;
in solution with a second peptide having the sequence:

[SEQ ID NO: 85]
Asp-Asn-Leu-Ala-Ala-Arg-Asp-Phe-Ile-Asn-Trp-
Leu-Ile-Gln-Thr-Lys-Ile-Thr-Asp-OH

wherein one or more of the amino acid residues in said first and second peptides may be unprotected or protected, preferably with an acid-cleavable protecting group;
(ii) optionally removing any protecting groups;
(iii) optionally purifying the GLP-2 peptide.

In one preferred embodiment the first peptide has the formula:

[SEQ ID NO: 86]
P1-His(P)-Gly-Asp(P)-Gly-Ser(P)-Phe-Ser(P)-
Asp(P)-Glu(P)-Met-Asn(P)-Thr(P)-Ile-Leu-OP2

wherein:
P1 is a protecting group for the N-terminal of His (preferably Boc, or Fmoc);
each P represents a side chain protecting group which may be the same or different; and
P2 is H or an activated carboxylic ester of the Leu residue (preferably Su, Bt or Pfp).

In one preferred embodiment, the GLP-2 peptide is of SEQ ID NO: 83, and the first peptide is Boc-His(Trt)-Gly-Asp(tBu)-Gly-Ser(tBu)-Phe-Ser(tBu)-Asp(tBu)-Glu(tBu)-Met-Asn(Trt)-Thr(tBu)-Ile-Leu-OH [SEQ ID NO: 87], where Phe-Ser(tBu)- can also be -Phe-ψSer-.

In another preferred embodiment, the GLP-2 peptide is of SEQ ID NO: 108, and the first peptide is Boc-His(Trt)¹-Aib-Asp(tBu)-Gly⁴-Ser(tBu)-Phe-Ser(tBu)-Asp(tBu)-Glu(tBu)-Met-Asn(Trt)-Thr(tBu)-Ile-Leu¹⁴-OH [(Boc-Aib²Ted(1-14)-OH], where Phe-Ser(tBu)- can also be -Phe-YSer-.

In one preferred embodiment the second peptide has the formula:

[SEQ ID NO: 88]
Asp(P)-Asn(P)-Leu-Ala-Ala-Arg(P)-Asp(P)-Phe-
Ile-Asn(P)-Trp(P)-Leu-Ile-Gln(P)-Thr(P)-
Lys(P)-Ile-Thr(P)-Asp(P)-O-P3

wherein each P represents a side chain protecting group which may be the same or different; and
P3 is H or a carboxy protecting group, preferably selected from Clt, Trt, tBu, DPM, MeDPM and MeODPM.

In one preferred embodiment the second peptide is selected from:

[SEQ ID NO: 89]
Asp(tBu)-Asn(Trt)-Leu-Ala-Ala-Arg(Pbf)-
Asp(tBu)-Phe-Ile-Asn(Trt)-Trp(Boc)-Leu-
Ile-Gln(Trt)-Thr(tBu)-Lys(Boc)-Ile-Thr(tBu)-
Asp(tBu)-OH;
and
H-Asp(tBu)¹⁵-Asn(Trt)-Leu-Ala-Ala-Arg(Pbf)-
Asp(tBu)-Phe-Ile-Asn(Trt)-Trp(Boc)-Leu-Ile-
Gln(Trt)-Thr(tBu)-Lys(Boc)-Ile-Thr(tBu)-
Asp(tBu)³³-O—R[(H-Ted(15-33)-O—R],
wherein
R = H, Clt, Dpm, tBu,
and where
-Gln(Trt)-Thr(tBu)- can also be -Gln(Trt)-ψThr-.

In one preferred embodiment the GLP-2 peptide or analogue or variant thereof is of SEQ ID NO: 83:

```
  1 2 3 4 5 6 7 8 9 10
His -Gly -Asp -Gly -Ser -Phe -Ser -Asp -Glu -Met
  11 12 13 14 15 16 17 18 19 20
-Asn -Thr -Ile -Leu -Asp -Asn -Leu -Ala -Ala -Arg
  21 22 23 24 25 26 27 28 29 30
-Asp -Phe -Ile -Asn -Trp -Leu -Ile -Gln -Thr -Lys
  31 32 33
-Ile -Thr -Asp
```

or a variant thereof,
and said process comprises:
(i) coupling a first peptide having the sequence:

[SEQ ID NO: 90]
His-Gly-Asp-Gly-Ser-Phe-Ser-Asp-Glu-Met-Asn-Thr-
Ile-Leu-Asp-Asn-Leu

wherein:
the N-terminal of His is optionally protected with a protecting group, preferably Boc, or Fmoc; and
   the Leu carboxylic acid group is optionally in the form of an activated carboxylic acid derivative;
in solution with a second peptide having the sequence:

[SEQ ID NO: 91]
Ala-Ala-Arg-Asp-Phe-Ile-Asn-Trp-Leu-Ile-Gln-Thr-
Lys-Ile-Thr-Asp-OH

wherein one or more of the amino acid residues in said first and second peptides may be unprotected or protected, preferably with an acid-cleavable protecting group;
(ii) optionally removing any protecting groups;
(iii) optionally purifying the GLP-2 peptide.

In one preferred embodiment the first peptide has the formula:

[SEQ ID NO: 92]
P1-His(P)-Gly-Asp(P)-Gly-Ser(P)-Phe-Ser(P)-
Asp(P)-Glu(P)-Met-Asn(P)-Thr(P)-Ile-Leu-Asp(P)-
Asn(P)-Leu-O-P2

wherein:
P1 is a protecting group for the N-terminal of His (preferably Boc, or Fmoc);
each P represents a side chain protecting group which may be the same or different; and
P2 is H or an activated carboxylic ester of the Leu residue (preferably Su, Bt or Pfp).

In one preferred embodiment, the GLP-2 peptide is of SEQ ID NO: 83, and the first peptide is Boc-His(Trt)-Gly-Asp(tBu)-Gly-Ser(tBu)-Phe-Ser(tBu)-Asp(tBu)-Glu(tBu)-Met-Asn(Trt)-Thr(tBu)-Ile-Leu-Asp(tBu)-Asn(Trt)-Leu-OH [SEQ ID NO: 93], where Phe-Ser(tBu)- can also be -Phe-ψSer-.

In another preferred embodiment, the GLP-2 peptide is of SEQ ID NO: 108, and the first peptide is Boc-His(Trt)¹-Aib-Asp(tBu)-Gly⁴-Ser(tBu)-Phe-Ser(tBu)-Asp(tBu)-Glu(tBu)-Met-Asn(Trt)-Thr(tBu)-Ile-Leu-Asp(tBu)-Asn(Trt)-Leu¹⁷-OH [(Boc-Aib2Ted (1-17)-OH], where Phe-Ser(tBu)- can also be -Phe-ψSer-.

In one preferred embodiment the second peptide has the formula:

[SEQ ID NO: 94]
Ala-Ala-Arg(P)-Asp(P)-Phe-Ile-Asn(P)-Trp(P)-Leu-
Ile-Gln(P)-Thr(P)-Lys(P)-Ile-Thr(P)-Asp(P)-O-P3

wherein each P represents a side chain protecting group which may be the same or different; and
P3 is H or a carboxy protecting group, preferably selected from Clt, Trt, tBu, DPM, MeDPM and MeODPM.

In one preferred embodiment the second peptide is selected from:

[SEQ ID NO: 95]
Ala-Ala-Arg(Pbf)-Asp(tBu)-Phe-Ile-Asn(Trt)-
Trp(Boc)-Leu-Ile-Gln(Trt)-Thr(tBu)-Lys(Boc)-Ile-
Thr(tBu)-Asp(tBu)-OH;
and
H-Ala¹⁸-Ala-Arg(Pbf)-Asp(tBu)-Phe-Ile-Asn(Trt)-
Trp(Boc)-Leu-Ile-Gln(Trt)-Thr(tBu)-Lys(Boc)-Ile-
Thr(tBu)-Asp(tBu)³³-O—R[(H-Ted(18-33)-O R],
wherein
R = H, Clt, Dpm, tBu, where
-Gln(Trt)-Thr(tBu)- can also be -Gln(Trt)-ψThr-.

In one preferred embodiment the GLP-2 peptide or analogue or variant thereof is of SEQ ID NO: 83:

```
  1 2 3 4 5 6 7 8 9 10
His -Gly -Asp -Gly -Ser -Phe -Ser -Asp -Glu -Met
  11 12 13 14 15 16 17 18 19 20
-Asn -Thr -Ile -Leu -Asp -Asn -Leu -Ala -Ala -Arg
  21 22 23 24 25 26 27 28 29 30
-Asp -Phe -Ile -Asn -Trp -Leu -Ile -Gln -Thr -Lys
  31 32 33
-Ile -Thr -Asp
```

or a variant thereof,
and said process comprises:
(i) coupling a first peptide having the sequence:

[SEQ ID NO: 96]
His-Gly-Asp-Gly-Ser-Phe-Ser-Asp-Glu-Met-Asn-Thr-
Ile-Leu-Asp-Asn-Leu-Ala

wherein:
the N-terminal of His is optionally protected with a protecting group, preferably Boc, or Fmoc; and
   the Ala carboxylic acid group is optionally in the form of an activated carboxylic acid derivative;
in solution with a second peptide having the sequence:

[SEQ ID NO: 97]
Ala-Arg-Asp-Phe-Ile-Asn-Trp-Leu-Ile-Gln-Thr-Lys-
Ile-Thr-Asp-OH

wherein one or more of the amino acid residues in said first and second peptides may be unprotected or protected, preferably with an acid-cleavable protecting group;
(ii) optionally removing any protecting groups;
(iii) optionally purifying the GLP-2 peptide.

In one preferred embodiment the first peptide has the formula:

[SEQ ID NO: 98]
P1-His(P)-Gly-Asp(P)-Gly-Ser(P)-Phe-Ser(P)-
Asp(P)-Glu(P)-Met-Asn(P)-Thr(P)-Ile-Leu-
Asp(P)-Asn(P)-Leu-Ala-O-P2

wherein:
P1 is a protecting group for the N-terminal of His (preferably Boc, or Fmoc);
each P represents a side chain protecting group which may be the same or different; and
P2 is H or an activated carboxylic ester of the Ala residue (preferably Su, Bt or Pfp).

In one preferred embodiment, the GLP-2 peptide is of SEQ ID NO: 83, and the first peptide is Boc-His(Trt)-Gly-Asp(tBu)-Gly-Ser(tBu)-Phe-Ser(tBu)-Asp(tBu)-Glu(tBu)-Met-Asn(Trt)-Thr(tBu)-Ile-Leu-Asp(tBu)-Asn(Trt)-Leu-Ala-OH [SEQ ID NO: 99], where Phe-Ser(tBu)- can also be -Phe-ψSer-.

In another preferred embodiment, the GLP-2 peptide is of SEQ ID NO: 108, and the first peptide is Boc-His(Trt)¹-Aib-Asp(tBu)-Gly⁴-Ser(tBu)-Phe-Ser(tBu)-Asp(tBu)-Glu(tBu)-Met-Asn(Trt)-Thr(tBu)-Ile-Leu-Asp(tBu)-Asn(Trt)-Leu-Ala¹⁸-OH [(Boc-Aib²Ted(1-18)-OH], where Phe-Ser(tBu)- can also be -Phe-ψSer-.

In one preferred embodiment the second peptide has the formula:

[SEQ ID NO: 100]
Ala-Arg(P)-Asp(P)-Phe-Ile-Asn(P)-Trp(P)-Leu-Ile-
Gln(P)-Thr(P)-Lys(P)-Ile-Thr(P)-Asp(P)-O-P3

wherein each P represents a side chain protecting group which may be the same or different; and
P3 is H or a carboxy protecting group, preferably selected from Clt, Trt, tBu, DPM, MeDPM and MeODPM.

In one preferred embodiment the second peptide is selected from:

[SEQ ID NO: 101]
Ala-Arg(Pbf)-Asp(tBu)-Phe-Ile-Asn(Trt)-Trp(Boc)-
Leu-Ile-Gln(Trt)-Thr(tBu)-Lys(Boc)-Ile-Thr(tBu)-
Asp(tBu)-OH;
and
H-Ala¹⁹-Arg(Pbf)-Asp(tBu)-Phe-Ile-Asn(Trt)-
Trp(Boc)-Leu-Ile-Gln(Trt)-Thr(tBu)-Lys(Boc)-
Ile-Thr(tBu)-Asp(tBu)³³-O—R[(H-Ted(19-33)-O—R],
wherein R = H, Clt, Dpm, tBu,
and where
-Gln(Trt)-Thr(tBu)-
can also be
-Gln(Trt)-ΨThr-.

In one preferred embodiment the GLP-2 peptide or analogue or variant thereof is of SEQ ID NO: 83:

1 2 3 4 5 6 7 8
His-Gly-Asp-Gly-Ser-Phe-Ser-Asp-
9 10 11 12 13 14 15
Glu-Met-Asn-Thr-Ile-Leu-Asp-
16 17 18 19 20 21 22 23
Asn-Leu-Ala-Ala-Arg-Asp-Phe-Ile-
24 25 26 27 28 29 30
Asn-Trp-Leu-Ile-Gln-Thr-Lys-
31 32 33
Ile-Thr-Asp

or a variant thereof,
and said process comprises:
(i) coupling a first peptide having the sequence:

[SEQ ID NO: 102]
His-Gly-Asp-Gly-Ser-Phe-Ser-Asp-Glu-Met-
Asn-Thr-Ile-Leu-Asp-Asn-Leu-Ala-Ala

wherein:
the N-terminal of His is optionally protected with a protecting group, preferably Boc, or Fmoc; and
   the Ala carboxylic acid group is optionally in the form of an activated carboxylic acid derivative;
in solution with a second peptide having the sequence:

[SEQ ID NO: 103]
Arg-Asp-Phe-Ile-Asn-Trp-Leu-Ile-Gln-
Thr-Lys-Ile-Thr-Asp-OH

wherein one or more of the amino acid residues in said first and second peptides may be unprotected or protected, preferably with an acid-cleavable protecting group;
(ii) optionally removing any protecting groups;
(iii) optionally purifying the GLP-2 peptide.

In one preferred embodiment the first peptide has the formula:

[SEQ ID NO: 104]
P1-His(P)-Gly-Asp(P)-Gly-Ser(P)-Phe-Ser(P)-
Asp(P)-Glu(P)-Met-Asn(P)-Thr(P)-Ile-Leu-
Asp(P)-Asn(P)-Leu-Ala-Ala-O-P2

wherein:
P1 is a protecting group for the N-terminal of His (preferably Boc, or Fmoc);
each P represents a side chain protecting group which may be the same or different; and
P2 is H or an activated carboxylic ester of the Ala residue (preferably Su, Bt or Pfp).

In one preferred embodiment, the GLP-2 peptide is of SEQ ID NO: 83 and the first peptide is Boc-His(Trt)-Gly-Asp(tBu)-Gly-Ser(tBu)-Phe-Ser(tBu)-Asp(tBu)-Glu(tBu)-Met-Asn(Trt)-Thr(tBu)-Ile-Leu-Asp(tBu)-Asn(Trt)-Leu-Ala-Ala-OH [SEQ ID NO: 105], where Phe-Ser(tBu)- can also be -Phe-ψSer-.

In one preferred embodiment, the GLP-2 peptide is of SEQ ID NO: 108 and the first peptide is Boc-His(Trt)¹-Aib-Asp(tBu)-Gly⁴-Ser(tBu)-Phe-Ser(tBu)-Asp(tBu)-Glu(tBu)-Met-Asn(Trt)-Thr(tBu)-Ile-Leu-Asp(tBu)-Asn(Trt)-Leu-Ala-Ala¹⁹-OH [(Boc-Aib²Ted(1-19)-OH], where Phe-Ser(tBu)- can also be -Phe-ψSer-.

In one preferred embodiment the second peptide has the formula:

[SEQ ID NO: 106]
Arg(P)-Asp(P)-Phe-Ile-Asn(P)-Trp(P)-Leu-Ile-
Gln(P)-Thr(P)-Lys(P)-Ile-Thr(P)-Asp(P)-O-P3

wherein each P represents a side chain protecting group which may be the same or different; and
P3 is H or a carboxy protecting group, preferably selected from Clt, Trt, tBu, DPM, MeDPM and MeODPM.

In one preferred embodiment the second peptide is selected from:

[SEQ ID NO: 107]
Arg(Pbf)-Asp(tBu)-Phe-Ile-Asn(Trt)-Trp(Boc)-Leu-
Ile-Gln(Trt)-Thr(tBu)-Lys(Boc)-Ile-Thr(tBu)-
Asp(tBu)-OH;
and
H-Arg(Pbf)²⁰-Asp(tBu)-Phe-Ile-Asn(Trt)-Trp(Boc)-
Leu-Ile-Gln(Trt)-Thr(tBu)-Lys(Boc)-Ile-Thr(tBu)-
Asp(tBu)³³-O—R[(H-Ted(20-33)-O—R],
wherein R = H, Clt, Dpm, tBu,
and where
-Gln(Trt)-Thr(tBu)-
can also be
-Gln(Trt)-ΨThr-.

Preferably, the first fragment is prepared from:
Boc-His(Trt)¹-Gly-Asp(OtBu)-Gly⁴-OH [(Boc-Ted(1-4)-OH]
Boc-His(Trt)¹-Aib-Asp(OtBu)-Gly⁴-OH [(Boc-Aib²Ted(1-4)-OH]
Boc-His(Trt)¹-Gly-Asp(OtBu)-Gly⁴-O-Pfp [(Boc-Ted(1-4)-O-Pfp]
Boc-His(Trt)¹-Aib-Asp(OtBu)-Gly⁴-O-Pfp [(Boc-Aib²Ted(1-4)-O-Pfp]
by coupling on solid-phase (R=2-chlorotrityl resin) or in solution (R=H) with one of the following fragments:
H-Ser(tBu)⁵-Phe-Ser(tBu)-Asp(OtBu)-Glu(OtBu)-Met-Asn(Trt)-Thr(tBu)-Ile-Leu¹⁴-O—R [H-Ted(5-14)-O—R]
H-Ser(tBu)⁵-Phe-Ser(tBu)-Asp(OtBu)-Glu(OtBu)-Met-Asn(Trt)-Thr(tBu)-Ile-Leu-Asp(OtBu)-Asn(Trt)-Leu¹⁷-O—R [H-Ted(5-17)-O—R]
H-Ser(tBu)⁵-Phe-Ser(tBu)-Asp(OtBu)-Glu(OtBu)-Met-Asn(Trt)-Thr(tBu)-Ile-Leu-Asp(OtBu)-Asn(Trt)-Leu-Ala¹⁸-O—R [H-Ted(5-18)-O—R]
H-Ser(tBu)⁵-Phe-Ser(tBu)-Asp(OtBu)-Glu(OtBu)-Met-Asn(Trt)-Thr(tBu)-Ile-Leu-Asp(OtBu)-Asn(Trt)-Leu-Ala-Ala¹⁹-O—R [H-Ted(5-19)-O—R]
to give a first fragment of Teduglutide (or its Aib²-analogue) as set out below:
Boc-His(Trt)1-Gly-Asp(tBu)-Gly-Ser(tBu)-Phe-Ser(tBu)-Asp(tBu)-Glu(tBu)-Met-Asn(Trt)-Thr(tBu)-Ile-Leu¹⁴-OH [Boc-Ted(1-14)-OH]
Boc-His(Trt)¹-Gly-Asp(tBu)-Gly-Ser(tBu)-Phe-Ser(tBu)-Asp(tBu)-Glu(tBu)-Met-Asn(Trt)-Thr(tBu)-Ile-Leu-Asp(tBu)-Asn(Trt)-Leu¹⁷-OH [Boc-Ted(1-17)-OH]
Boc-His(Trt)¹-Gly-Asp(tBu)-Gly-Ser(tBu)-Phe-Ser(tBu)-Asp(tBu)-Glu(tBu)-Met-Asn(Trt)-Thr(tBu)-Ile-Leu-Asp(tBu)-Asn(Trt)-Leu-Ala¹⁸-OH [Boc-Ted(1-18)-OH]
Boc-His(Trt)¹-Gly-Asp(tBu)-Gly-Ser(tBu)-Phe-Ser(tBu)-Asp(tBu)-Glu(tBu)-Met-Asn(Trt)-Thr(tBu)-Ile-Leu-Asp(tBu)-Asn(Trt)-Leu-Ala-Ala¹⁹-OH [Boc-Ted(1-19)-OH]
Boc-His(Trt)¹-Aib-Asp(tBu)-Gly4-Ser(tBu)-Phe-Ser(tBu)-Asp(tBu)-Glu(tBu)-Met-Asn(Trt)-Thr(tBu)-Ile-Leu¹⁴-OH [(Boc-Aib²Ted(1-14)-OH]
Boc-His(Trt)¹-Aib-Asp(tBu)-Gly⁴-Ser(tBu)-Phe-Ser(tBu)-Asp(tBu)-Glu(tBu)-Met-Asn(Trt)-Thr(tBu)-Ile-Leu-Asp(tBu)-Asn(Trt)-Leu¹⁷-OH [(Boc-Aib²Ted(1-17)-OH]
Boc-His(Trt)¹-Aib-Asp(tBu)-Gly⁴-Ser(tBu)-Phe-Ser(tBu)-Asp(tBu)-Glu(tBu)-Met-Asn(Trt)-Thr(tBu)-Ile-Leu-Asp(tBu)-Asn(Trt)-Leu-Ala¹⁸-OH [(Boc-Aib²Ted(1-18)-OH]
Boc-His(Trt)¹-Aib-Asp(tBu)-Gly⁴-Ser(tBu)-Phe-Ser(tBu)-Asp(tBu)-Glu(tBu)-Met-Asn(Trt)-Thr(tBu)-Ile-Leu-Asp(tBu)-Asn(Trt)-Leu-Ala-Ala¹⁹-OH [(Boc-Aib²Ted(1-19)-OH]
(where Phe-Ser(tBu)- can also be -Phe-ψ′Ser-).

Preferably, the first fragment of Teduglutide obtained above is then coupled with the corresponding second fragment selected from:
H-Asp(tBu)¹⁵-Asn(Trt)-Leu-Ala-Ala-Arg(Pbf)-Asp(tBu)-Phe-Ile-Asn(Trt)-Trp(Boc)-Leu-Ile-Gln(Trt)-Thr(tBu)-Lys(Boc)-Ile-Thr(tBu)-Asp(tBu)³³-O—R [(H-Ted(15-33)-O—R],
wherein R=H, Clt, Dpm, tBu as described for Liraglutide and Semaglutide;
H-Ala¹⁸-Ala-Arg(Pbf)-Asp(tBu)-Phe-Ile-Asn(Trt)-Trp(Boc)-Leu-Ile-Gln(Trt)-Thr(tBu)-Lys(Boc)-Ile-Thr(tBu)-Asp(tBu)³³-O—R [(H-Ted(18-33)-O—R];
H-Ala¹⁹-Arg(Pbf)-Asp(tBu)-Phe-Ile-Asn(Trt)-Trp(Boc)-Leu-Ile-Gln(Trt)-Thr(tBu)-Lys(Boc)-Ile-Thr(tBu)-Asp(tBu)³³-O—R [(H-Ted(19-33)-O—R];
H-Arg(Pbf)²⁰-Asp(tBu)-Phe-Ile-Asn(Trt)-Trp(Boc)-Leu-Ile-Gln(Trt)-Thr(tBu)-Lys(Boc)-Ile-Thr(tBu)-Asp(tBu)³³-O—R [(H-Ted(20-33)-O—R];
where R=2-chlorotrityl resin or H, and where -Gln(Trt)-Thr(tBu)- can also be -Gln(Trt)-ψThr-
to form Teduglutide (1-33).

For all the embodiments described herein, preferably, the first fragment is prepared on solid phase or in solution. Where the first fragment is prepared on solid phase, it is cleaved from the resin before coupling with the second fragment in solution.

For all the embodiments described herein, preferably the second fragment is prepared on solid phase or in solution. Where the second fragment is prepared on solid phase, it is cleaved from the resin before coupling with the first fragment in solution.

For all the embodiments described herein, preferably the second fragment is prepared by coupling two or more sub-fragments.

In one preferred embodiment, the crude GLP peptide is purified by preparative HPLC using various buffers in water/acetonitrile or water/methanol.

In another preferred embodiment, the crude GLP peptide is purified by reverse phase chromatography, for example, reverse phase HPLC.

In one preferred embodiment, the reverse phase chromatography is carried out using C18, C8 or C4 modified silica, for example, “RP-18” (octadecyl carbon chain C18-bonded silica), or “RP-82 (C8-bonded silica). Suitable mobile phases are as described in WO 2016/046753. For example, in one embodiment, the crude peptide is subjected to reverse phase chromatography on a C8 or C8 column using a mobile phase A, comprising water, and a mobile phase B, comprising acetonitrile and at least one C₁₋₄-alcohol, and optionally repeating. The resulting fractions are then subjected to reverse phase chromatography on a C8 or C8 column using a mobile phase C, comprising water, and a mobile phase D, comprising acetonitrile, and optionally repeating. The resulting fractions are then dried.

In one preferred embodiment, the crude peptides were first treated with RP-18 or RP-8 material in a buffer (RP-18=octadecyl carbon chain, C18-bonded silica; RP-8=C8-bonded silica) where early eluting impurities remained in solution while the required peptides remained bound on the silica. After washing the RP-material with this buffer, the RP-material was treated with a buffer that elutes >95% of the main product but retains the more lypophilic impurities on the silica. By this method >90-95% crude Liraglutide and Semaglutide and >80% crude Teduglutide were obtained and subjected to usual HPLC purification with RP-4, RP-8 or RP-18 reverse phase silica. Among other buffers ammonium acetate or ammonium carbonate or ammonium formate were used preferentially giving products of >99% purity with no single impurity exceeding the 0.15%. Such products are well suited to be used in pharmaceutical preparations.

For Liraglutide, in one preferred embodiment, the second peptide is prepared by solid phase synthesis using Lys(Pal-Glu-O^{t}Bu)-OH. In a more preferred embodiment, the second peptide is prepared by the steps of (i) solid phase synthesis solid phase synthesis using Fmoc-Lys(Mmt)-OH, (ii) cleaving from the resin and simultaneously removing the Mmt group from the Lys side chain, and (iii) treating with Pal-Glu(OSu)O^{t}Bu or Pal-Glu(OPfp)O^{t}Bu, (iv) esterifying in solution with a trityl type group, a diphenylmethyl group or a tert-butyl group.

For Semglutide, in one preferred embodiment, the second peptide is prepared by solid phase synthesis using Lys(C18-Glu-PEG2)-OH. In a more preferred embodiment, the second peptide is prepared by the steps of (i) solid phase synthesis solid phase synthesis using Fmoc-Lys(Mmt)-OH, (ii) cleaving from the resin and simultaneously removing the Mmt group from the Lys side chain, and (iii) treating with C18-Glu(OSu)-PEG2 or C18-Glu(OPfp)-PEG2, (iv) esterifying in solution with a trityl type group, a diphenylmethyl group or a tert-butyl group.

In one preferred embodiment, the peptide of [SEQ ID NO: 2] is prepared from His-Ala-Glu-Gly [SEQ ID NO: 51] by stepwise solid phase synthesis.

In a more preferred embodiment, the peptide of [SEQ ID NO: 2] is prepared by fragment condensation of P1-His-Ala-Glu-Gly-OH [SEQ ID NO: 52] and H-Thr-Phe-Thr-Ser-Asp-Val-Ser-Ser-Tyr-Leu-Glu-Gly-Gln-Ala-Ala-O-P3 [SEQ ID NO: 53], wherein P1 is a protecting group for the N-terminal, and P3 is a protecting group for the C-terminal, and where one or more amino acid residues in the peptides may be unprotected or protected.

In one preferred embodiment, the peptide of [SEQ ID NO: 9] is prepared from His-Aib-Glu-Gly [SEQ ID NO: 54] by stepwise solid phase synthesis. In a more preferred embodiment, the peptide of [SEQ ID NO: 9] is prepared by fragment condensation of P1-His-Aib-Glu-Gly-OH [SEQ ID NO: 55] and H-Thr-Phe-Thr-Ser-Asp-Val-Ser-Ser-Tyr-Leu-Glu-Gly-Gln-Ala-Ala-O-P3 [SEQ ID NO: 56], wherein P1 is a protecting group for the N-terminal, and P3 is a protecting group for the C-terminal, and where one or more amino acid residues in the peptides may be unprotected or protected.

In one preferred embodiment, the GLP-1 peptide or analogue or variant thereof is of SEQ ID NO: 1:

1 2 3 4 5 6 7 8 9 10 11
His-Ala-Glu-Gly-Thr-Phe-Thr-Ser-Asp-Val-Ser-
12 13 14 15 16 17 18 19
Ser-Tyr-Leu-Glu-Gly-Gln-Ala-Ala-
20 21 22 23 24 25 26 27 28
Lys(Pal-Glu)-Glu-Phe-Ile-Ala-Trp-Leu-Val-Arg-
29 30 31
Gly-Arg-Gly-OH

and said process comprises:
   (i) coupling a first peptide having the sequence:

[SEQ ID NO: 57]
Xₙ . . . X₁₈-Ala

wherein:
Xₙ . . . X₁₈ represents amino acid residues n to 18 of liraglutide, where n is 1 to 17;
   the N-terminal is optionally protected with a protecting group, preferably Boc, or Fmoc; and
   the Ala carboxylic acid group is optionally in the form of an activated carboxylic acid derivative;
in solution with a second peptide having the sequence:

[SEQ ID NO: 3]
Lys(Pal-Glu-OX)-Glu-Phe-Ile-Ala-Trp-
Leu-Val-Arg-Gly-Arg-Gly-OH

wherein:
   X is H or a protecting group for the Glu carboxylic acid group; and wherein one or more of the amino acid residues in the first and second peptides can be unprotected or protected, preferably with an acid-cleavable protecting group;
(ii) optionally removing any protecting groups;
(iii) optionally purifying the GLP-1 peptide.

In one preferred embodiment, n is 2 to 17, and wherein after solution phase coupling with SEQ ID NO:3, said process further comprises the stepwise addition of one or more amino acids, or condensation with a sub-fragment, to give SEQ ID NO: 1.

In one particularly preferred embodiment, n is 15.

In one preferred embodiment, the GLP-1 peptide or analogue or variant thereof is of SEQ ID NO: 8:

1 2 3 4 5 6 7 8 9 10
His-Aib-Glu-Gly-Thr-Phe-Thr-Ser-Asp-Val-
11 12 13 14 15 16 17 18 19
Ser-Ser-Tyr-Leu-Glu-Gly-Gln-Ala-Ala-
20 21 22 23 24 25 26 27
Lys(W)-Glu-Phe-Ile-Ala-Trp-Leu-Val-
28 29 30 31
Arg-Gly-Arg-Gly-OH

where W is N-(17-carboxy-1-oxoheptadecyl)-L-γ-glutamyl-2-[2-(2-aminoethoxy) ethoxy]acetyl-2-[2-(2-aminoethoxy)ethoxy]acetyl;
and said process comprises:
   (i) coupling a first peptide having the sequence:

[SEQ ID NO: 58]
Yₙ . . . Y₁₈-Ala

wherein:
   Yₙ . . . Y₁₈ represents amino acid residues n to 18 of semaglutide, where n is 1 to 17;
   the N-terminal is optionally protected with a protecting group, preferably Boc, or Fmoc; and
   the Ala carboxylic acid group is optionally in the form of an activated carboxylic acid derivative;
   in solution with a second peptide having the sequence:

[SEQ ID NO: 10]
Lys(W1)-Glu-Phe-Ile-Ala-Trp-Leu-Val-
Arg-Gly-Arg-Gly-OH

wherein:
   W1 is N-(17-carboxy-1-oxoheptadecyl)-L-γ-glutamyl-2-[2-(2-aminoethoxy) ethoxy]acetyl-2-[2-(2-aminoethoxy)ethoxy]acetyl;
   and wherein one or more of the amino acid residues in said first and second peptides and W1 may be unprotected or protected, preferably with an acid-cleavable protecting group;
(ii) optionally removing any protecting groups;
(iii) optionally purifying the GLP-1 peptide.

In one preferred embodiment, n is 2 to 17, and wherein after solution phase coupling with SEQ ID NO: 10, said process further comprises the stepwise addition of one or more amino acids, or condensation with a sub-fragment, to give SEQ ID NO: 8.

In one particularly preferred embodiment, n is 15.

Another aspect of the invention relates to one or more fragments as described, for example, a peptide selected from SEQ ID NOS: 2-7 and 9-58.

Another aspect of the invention relates to the use of one or more peptide fragments as described herein in the synthesis of a GLP-1 peptide or analogue or variant thereof, more preferably, Liraglutide or Semaglutide. In one preferred embodiment, the invention relates to the use of a peptide selected from SEQ ID NOS: 2-7 and 9-58 in the synthesis of a GLP-1 peptide.

Another aspect of the invention relates to the use of one or more peptide fragments as described herein in the synthesis of a GLP-2 peptide or analogue or variant thereof, more preferably, Teuglutide. In one preferred embodiment, the invention relates to the use of a peptide selected from SEQ ID NOS: 84-107 in the synthesis of a GLP-2 peptide.

The present invention is further described by way of the following non-limiting examples.

### EXAMPLES

### Abbreviations

AIB (or Aib) 2-Aminoisobutyric acid or α-aminoisobutyric acid

Boc or t-Boc t-butyloxycarbonyl

Bt benzotriazole

carboxybenzyl

Bz benzyl

Dde 1-(4,4-Dimethyl-2,6-dioxocyclohex-1-ylidene)ethyl

EDC·HCl 1-ethyl-3-(3 -dimethyl-aminopropyl)carbodiimide hydrochloride #

DPM diphenylmethyl

MeDPM methyl-diphenylmethyl

MeODPM methoxy-diphenylmethyl

ivDde 1-(4,4-Dimethyl-2,6-dioxocyclohexylidene)-3-methylbutyl

Fmoc 9-fluorenylmethoxycarbonyl

HPLC High Performance Liquid Chromatography

Mmt monomethoxytrityl [(4-methoxyphenyl)diphenylmethyl]

Mtt 4-methyltrityl

Pfp pentafluorophenyl

Su succinimide

tBu tert-butyl

Pal palmitoyl

Pbf 2,2,4,6,7-pentamethyl-dihydrobenzofuran-5-sulfonyl

TFA trifluoroacetic acid

Trt trityl

Clt chlorotrityl

### Materials & Methods

The (1-19), (1-18) and (1-17) fragments of Liraglutide/Semaglutide were obtained either by the step-by-step method on 2-chlorotrityl resin using Fmoc-amino acids or by the fragment condensation method.

Resin-bound fragments (20-31), (19-31), (18-31) of Liraglutide/Semaglutide etc were synthesized by the step by step method on 2-chlorotrityl resin or the Wang resin using Fmoc-Lys²⁰(Pal-Glu-OtBu)-OH or Fmoc-Lys(C18-Glu-PEG2)-OH. Alternatively, the fragments were prepared by introducing Lys(20) with Fmoc-Lys(Mmt)-OH on 2-chlorotrityl resin. The fragments were then cleaved from the resin with simultaneous removal of the Mmt group from the side chain of Lys(20). Pal-Glu or C18-Glu-PEG2 was then incorporated using Fmoc or C18-Glu(OSu)-PEG2 or the corresponding Pfp derivatives. The formed Lys²⁰(Pal-Glu-OtBu) or Lys²⁰ (C18Glu-PEG2)20-31 fragments were then esterified in solution using a suitable method with a trityl type group, a diphenyl methyl type group or with ^{t}Bu.

The protected esters ((20-31), (19-31), (18-31) etc) were then condensed in solution with the L-Ala(1-19), L-Ala(1-18), L-Gln(1-17) protected fragments using methods known in the art. Dehydrating agents such as EDAC/DIPEA, DIC, HBTU and an acidic catalyst such as HOBt, HOAtu, PfpOH are used to facilitate the condensation reaction. Similar methods were used to prepare the fragments of Teduglutide.

### General Procedures for the Preparation of Protected Fragments

### Resin Loading

CLTR-Cl is charged to a peptide reactor and swelled with DCM for 10 min at RT. The resin is drained and a solution of Fmoc-amino acid and DIEA in DCM is added. The mixture is stirred under nitrogen for 2 hours at RT. Then, remaining active sites on CLTR are end-capped with addition of MeOH and stirring for 1 hour. The resin is drained, washed three times, 5 min each, with a mixture of DCM/DIEA/MeOH (80:5:15) and twice with NMP. After draining, the resin is treated twice with 15% piperidine in NMP for 30 min and then four times with NMP, four times with IPA, drained and dried to a constant weight in vacuum. A loading of 0.3 mmol/g resin was obtained.

### Coupling of the Second Amino Acid

The resin-bound amino acid is washed with NMP (3×6 mL/g resin) and drained. Then a mixture of preactivated Fmoc-amino acid with HBTU/HOBt/DIEA in the 3:3:3:6 molar ratio over the resin-bound amino acid in NMP (0.6M) is added. The mixture is shaken until negative Kaiser test, drained and washed with NMP (5×6 mL/g resin).

### Chain Elongation

The resin-bound peptide is reacted with 0.6M solution in NMP of 2.5 fold molar excess of the Fmoc-amino acid with respect to the resin-bound peptide. The amino acid was preactivated for 10 min at 0° C. and 10 min at 15° C. with DIC/HOBt in a 1.05:1.2 molar ratio in relation to the applied Fmoc-amino acid. After each coupling, the resin is washed with NMP and reaction completion is verified with Kaiser test.

### Cleavage of the Protected Peptides from the Resin

After the completion of the chain assembly, the resin-bound peptides are washed with DCM (×10). The resin is treated twice with 2% TFA in DCM and the resin is washed with DCM (×5). The combined filtrates are extracted with water (×5), concentrated and protected peptides are precipitated.

### Chlorotrityl Chloride Protection of C-Terminal Glycine of Fmoc-(20-31)-OH of Liraglutide

The Fmoc-(20-31)-OH protected peptide (1g, 0.365 μmol) is dissolved in 62 mL DCM under stirring and 2-CLT chloride (0.571 g, 1.825 μmol) is added. The reaction is left for 10 min and DIPEA (0.629 mL, 3.65 μmol) is added. The reaction is left to stand for 4 h. The DCM solution is concentrated and precipitated with diethyl ether (30 mL). The protected peptided is washed with ether (6×10 mL).

### Chlorotrityl Chloride Protection of C-Terminal Glycine of Fmoc-(20-31)-OH of Semaglutide

The Fmoc-(20-31)-OH protected peptide (5g, 1.27 mmol) is dissolved in 300 mL DCM under stirring and 2-CLT chloride (2g, 6.35 mmol) is added. The reaction is left for 10 min and DIPEA (2.2 mL, 12.7 mmol) is added. The reaction is left to stand for 4 h. The DCM solution is concentrated and precipitated with diethyl ether (120 mL). The protected peptided is washed with ether (6×50 mL).

### N^{a} Deprotection of Fmoc-(20-31)-O-Clt of Liraglutide

The protected peptide (0.9 g, 0.298 μmol) is dissolved in NMP (30 mL). After 10 min, piperidine (0.147 mL, 1.49 μmol) is added under stirring for 2.5 h. To the resulting solution DCM (90 mL) is added and the mixture is exctracted with water (8×100 mL). The final DCM-peptide solution is concentrated and precipitated.

### N^{a} Deprotection of Fmoc-(20-31)-O-Clt of Semaglutide

The protected peptide (5.4 g, 1.27 mmol) is dissolved in NMP (120 mL). After 10 min, piperidine (0.76 mL, 7.62 mmol) is added under stirring for 2.5 h. To the resulting solution DCM (360 mL) is added and the mixture is exctracted with water (8×350 mL). The final DCM-peptide solution is concentrated and precipitated.

### Liquid Phase Fragment Condensation of Boc-(1-19)-OH and H-(20-31)-O-Clt of Liraglutide

The protected peptide H-(20-31)-O-CLt (160 mg, 56 μmol) is dissolved in 7.5 mL NMP and cooled down to 5° C. The protected peptide Boc-(1-19)-OH (195 mg, 64 μmol) is dissolved in 2.5 mL NMP and HOBt·H₂O (11.8 mg, 77 μmol) is added. The resulting solution is cooled down to 5° C. and EDAC·HCl (13.4 mg, 70 μmol) is added. The solutions containing the protected fragments are mixed together under stirring for 1 h and then 13.4 mg EDAC·HCl (70 μmol) and DIPEA (10λ, 58 μmol) is added. The reaction is left under stirring for 20 h at ambient conditions. DCM (30 mL) is added and the organic phase is exctracted 5 times with water. The DCM solution is concentrated, precipitated and washed with Hexane (3×10 mL).

### Liquid Phase Fragment Condensation of Boc-(1-19)-OH and H-(20-31)-O-Clt of Semaglutide

The protected peptide H-(20-31)-O-CLt (3 g, 0.75 mmol) is dissolved in 130 mL NMP and cooled down to 5° C. The protected peptide Boc-(1-19)-OH (2.65 g, 0.86 μmol) is dissolved in 32 mL NMP and HOBt·H2O (158.0 mg, 1.32 mmol) is added. The resulting solution is cooled down to 5° C. and EDAC·HCl (181.3 mg, 0.94 mmol) is added. The solutions containing the protected fragments are mixed together under stirring for 1 h and then 181.3 mg EDAC·HCl (0.94 mmol) and DIPEA (129λ, 0.75 mmol) is added. The reaction is left under stirring for 20 h at ambient conditions. DCM (480 mL) is added and the organic phase is exctracted 5 times with water. The DCM solution is concentrated, precipitated and washed with Hexane (3×180 mL).

One highly preferred embodiment of the invention involves the condensation of the 1-19 fragment (Fragment 1) with the 20-31-OtBu Fragment 2. For example:

### For Liraglutide

[SEQ ID NO: 62]
Boc-His(Trt)-Ala-Glu(tBu)-Gly-Thr(tBu)-Phe-ΨThr-
Ser(tBu)-Asp(tBu)-Val-Ser(tBu)-ΨSer-Tyr(tBu)-Leu-
Glu(tBu)-Gly-Gln(Trt)-Ala-Ala-OH,
(where ΨSer and ΨThr is a pseudoproline) +
[SEQ ID NO: 63]
H-Lys(Pal-Glu)-Glu(tBu)-Phe-Ile-Ala-Trp(Boc)-Leu-
Val-Arg(Pbf)-Gly-Arg(Pbf)-Gly-O-tBu.

### For Semaglutide

[SEQ ID NO: 61]
Boc-His(Trt)-Aib-Glu(tBu)-Gly-Thr(tBu)-Phe-ΨThr-
Ser(tBu)-Asp(tBu)-Val-Ser(tBu)-ΨSer-Tyr(tBu)-Leu-
Glu(tBu)-Gly-Gln(Trt)-Ala-Ala-OH,
(where ΨSer and ΨThr is a pseudoproline) +
[SEQ ID NO: 64]
H-Lys(W)-Glu(tBu)-Phe-Ile-Ala-Trp(Boc)-Leu-Val-
Arg(Pbf)-Gly-Arg(Pbf)-Gly-O-tBu,
where W is N-(17-carboxy-1-oxoheptadecyl)-L-γ-
glutamyl-2-[2-(2-aminoethoxy)ethoxy]acetyl-2-
[2-(2-aminoethoxy)ethoxy]acetyl.

### Preparation of 20-31-OtBu Fragment

The 20-31-OtBu fragment was prepared by the condensation of the 20-29 Fragment+30-31 Fragment.

### Synthesis of the 20-31 Fragment 2 of Liraglutide

Fmoc-Lys(Pal-Glu)-Glu(tBu)-Phe-Ile-Ala-Trp(Boc)-Leu-Val-Arg(Pbf)-Gly-OH+H-Arg(Pbf)-Gly-O-tBu followed by Fmoc-removal of the product.

### Synthesis of the 20-31 Fragment 2 of Semaglutide

Fmoc-Lys(W)-Glu(tBu)-Phe-Ile-Ala-Trp(Boc)-Leu-Val-Arg(Pbf)-Gly-OH+H-Arg(Pbf)-Gly-O-tBu, where W is N-(17-carboxy-1-oxoheptadecyl)-L-γ-glutamyl-2-[2-(2-aminoethoxy) ethoxy] acetyl-2-[2-(2-aminoethoxy)ethoxy] acetyl, followed by Fmoc-removal of the product.

### Side-Chain Deprotection

To 75 mg of the final protected peptides a mixture of TFA/H₂O/DTT (3 mL) in a molar ratio of 94:3:3 is added at ambient conditions for 3 h. The TFA solution is concentrated and precipitated with prechilled diethylether 2 mL. The globally deprotected peptide is washed with DEE (5×0.5 mL) and left to dry to a constant weight.

### Purification

Crude Liraglutide, Semaglutide and Teduglutide can be purified by preparative HPLC using various buffers in water/acetonitrile or water/methanol. In one preferred embodiment, Liraglutide, Semaglutide and Teduglutide were purified in >99.0% purity applying buffers of ammonium acetate at pH 6.5-9.5, of tetrabutyl ammonium hydraxide at pH 7.0-9.5, of ammonium formate at pH 6.5-9.5 and of 0.5-3% acetic acid. Preferably 1% acetic acid and ammonium formate were used as the buffers. The overall purification yield was 70-90% and the total yield was 40-75%.

In one preferred embodiment, the crude peptides were first treated with RP-18 or RP-8 material in a buffer (RP-18=octadecyl carbon chain, C18-bonded silica; RP-8=C8-bonded silica) where early eluting impurities remained in solution while the required peptides remained bound on the silica. After washing the RP-material with this buffer, the RP-material was treated with a buffer that elutes >95% of the main product but retains the more lypophilic impurities on the silica. By this method >90-95% crude Liraglutide and Semaglutide and >80% crude Teduglutide were obtained and subjected to usual HPLC purification with RP-4, RP-8 or RP-18 reverse phase silica. Among other buffers ammonium acetate or ammonium carbonate or ammonium formate were used preferentially giving products of >99% purity with no single impurity exceeding the 0.15%. Such products are well suited to be used in pharmaceutical preparations.

For the purification of the crude peptides preparative HPLC Knauer K1800 was used with a column of 50 mm packed with Kromasil C18, 13 μm, 100 A.

A two step approach either with basic and acidic conditions and acetonitrile as the organic modifier, at ambient temperature was used for the preparation of highly pure peptides.

### Results

A summary of the results is given in Table 1. As expected, the condensations performed on solid-phase, using either the Wang or the 2-chlorotrityl resin proceeded with extensive racemization. In this regard, it was determined by HPLC that >33% D-isomer was formed in all cases, even though the condensation conditions were chosen to be as mild as possible. Lowering the condensation temperature led to a similar degree of racemization to when the condensation was performed at room temperature. However, when carried out in solution, surprisingly at several positions the racemization proved to be much lower (<7%) than expected compared to the condensation results on solid-phase. In particular at the positions Ala(19), Ala(18), Gln(17), and Leu(14) the observed racemization was less than 3%.

In addition to that observation, many of the diastereomeric D-peptides were surprisingly well separated from the corresponding L-diastereomers using analytical and preparative HPLC. Accordingly, the formed diastereomers could be easily separated from the main product.

The results for Teduglutide are shown in Table 2.

### Semaglutide and Liraglutide

The following fragments were prepared by methods described herein:
Boc-His(Trt)¹-Ala²-Glu(tBu)-Gly-Thr(tBu)-Phe-Thr(tBu)-Ser(tBu)-Asp(tBu)-Val-Ser(tBu)-Ser(tBu)-Tyr(tBu)-Leu-Glu(tBu)-Gly-Gln(Trt) -Ala-Ala-Lys(W)-Glu(tBu)-Phe-Ile-Ala-Trp(Boc)-Leu-Val-Arg(Pbf)-Gly-Arg(Pbf)³¹-Gly-Y
Y=OH, NH₂

### Acids

Boc-His(Trt)¹-Ala²-Glu(tBu)-Gly-OH (1-4)
Boc-His(Trt)¹-Aib²-Glu(tBu)-Gly-OH (1-4)
Boc-His(Trt)¹-Ala²-Glu(tBu)-Gly-Thr(tBu)-Phe-Thr(tBu)-Ser(tBu)-Asp(tBu)-Val-Ser(tBu)-Ser(tBu)-Tyr(tBu)-Leu-Glu(tBu)-Gly-Gln(Trt)-Ala-Ala-OH (1-19)
Boc-His(Trt)¹-Aib²-Glu(tBu)-Gly-Thr(tBu)-Phe-Thr(tBu)-Ser(tBu)-Asp(tBu)-Val-Ser(tBu)-Ser(tBu)-Tyr(tBu)-Leu-Glu(tBu)-Gly-Gln(Trt)-Ala-Ala-OH (1-19)
Boc-His(Trt)¹-Ala²-Glu(tBu)-Gly-Thr(tBu)-Phe-Thr(tBu)-Ser(tBu)-Asp(tBu)-Val-Ser(tBu)-Ser(tBu)-Tyr(tBu)-Leu-Glu(tBu)-Gly-Gln(Trt)-Ala-OH (1-18)
Boc-His(Trt)¹-Aib²-Glu(tBu)-Gly-Thr(tBu)-Phe-Thr(tBu)-Ser(tBu)-Asp(tBu)-Val-Ser(tBu)-Ser(tBu)-Tyr(tBu)-Leu-Glu(tBu)-Gly-Gln(Trt)-Ala-OH (1-18)
Boc-His(Trt)¹-Ala²-Glu(tBu)-Gly-Thr(tBu)-Phe-Thr(tBu)-Ser(tBu)-Asp(tBu)-Val-Ser(tBu)-Ser(tBu)-Tyr(tBu)-Leu-Glu(tBu)-Gly-Gln-OH (1-17)
Boc-His(Trt)¹-Aib²-Glu(tBu)-Gly-Thr(tBu)-Phe-Thr(tBu)-Ser(tBu)-Asp(tBu)-Val-Ser(tBu)-Ser(tBu)-Tyr(tBu)-Leu-Glu(tBu)-Gly-Gln(Trt)-OH (1-17)
Boc-His(Trt)¹-Aib²-Glu(tBu)-Gly-Thr(tBu)-Phe-Thr(tBu)-Ser(tBu)-Asp(tBu)-Val-Ser(tBu)-Ser(tBu)-Tyr(tBu)-Leu-Glu(tBu)-Gly-Gln-OH (1-17)
Fmoc-Gln(Trt)-Ala-Ala-OH(17-19)
Fmoc-Glu(tBu)-Gly-Gln(Trt)-Ala-Ala-OH (15-19)
Boc-His(Trt)¹-Ala²-Glu(tBu)-Gly-Thr(tBu)-Phe-Thr(tBu)-Ser(tBu)-Asp(tBu)-Val-Ser(tBu)-Ser(tBu)-Tyr(tBu)-Leu¹⁴-OH (1-14)
Boc-His(Trt)¹-Aib-Glu(tBu)-Gly-Thr(tBu)-Phe-Thr(tBu)-Ser(tBu)-Asp(tBu)-Val-Ser(tBu)-Ser(tBu)-Tyr(tBu)-Leu¹⁴-OH (1-14)
Fmoc-Glu(tBu)²¹-Phe-Ile-Ala-Trp(Boc)-Leu-Val-Arg(Pbf)-Gly-Arg(Pbf)³¹-OH (21-31) Precursor fragment used to make 15-31+(15-20)
Fmoc-Glu(tBu)¹⁵-Gly-Gln(Trt)-Ala-Ala-Lys(W)²⁰-OH (15-20) Precursor fragment used to make 15-31 (+21-31)
Fmoc-Glu(tBu)¹⁵-Gly-Gln(Trt)-Ala-Ala-Lys(W)-Glu(tBu)-Phe-Ile-Ala-Trp(Boc)-Leu-Val-Arg(Pbf)-Gly-Arg(Pbf)³¹-OH (15-31)

### Esters

H-Lys(W)-Glu(tBu)-Phe-Ile-Ala-Trp(Boc)-Leu-Val-Arg(Pbf)-Gly-Arg(Pbf)³¹-Gly-O-Pr₁ (20-31)
H-Ala-Lys(W)-Glu(tBu)-Phe-Ile-Ala-Trp(Boc)-Leu-Val-Arg(Pbf)-Gly-Arg(Pbf)³¹-Gly-O-Pr₁ (19-31)
H-Ala-Ala-Lys(W)-Glu(tBu)-Phe-Ile-Ala-Trp(Boc)-Leu-Val-Arg(Pbf)-Gly-Arg(Pbf)³¹-Gly-O-Pr₁ (18-31)
H-Gln(Trt)-Ala-Ala-Lys(W)-Glu(tBu)-Phe-Ile-Ala-Trp(Boc)-Leu-Val-Arg(Pbf)-Gly-Arg(Pbf)³¹-Gly-O-Pr₁ (17-31)
H-Glu(tBu)¹⁵-Gly-Gln(Trt)-Ala-Ala-Lys(W)-Glu(tBu)-Phe-Ile-Ala-Trp(Boc)-Leu-Val-Arg(Pbf)-Gly-Arg(Pbf)³¹-O Pr₁ (15-31)
Pr₁=Clt, Trt, tBu, DPM, MeDPM, MeODPM etc.

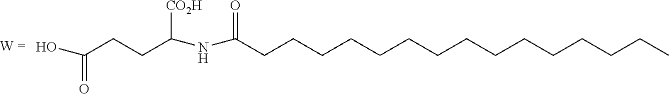 [SMILES: CCCCCCCCCCCCCCCC(=O)NC(CCC(=O)O)C(=O)O]

and Semaglutide by the 9, 18,23-trioxo-2,5, 11, 14-tetraoxa-8, 17,22-triazanonatriacontane-1,21,39-tricarboxylic acid.

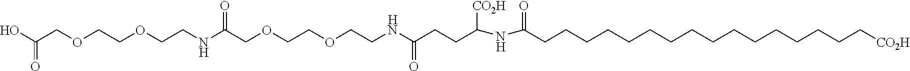 [SMILES: O=C(O)CCCCCCCCCCCCCCCCC(=O)NC(CCC(=O)NCCOCCOCC(=O)NCCOCCOCC(=O)O)C(=O)O]

See Table 1 for results.

### Teduglutide Acids (Synthesis of the First Fragment)

A protected (1-4) fragment of Teduglutide (or its Aib² analogue) selected from the following:
Boc-His(Trt)¹-Gly-Asp (OtBu)-Gly⁴-OH [(Boc-Ted(1-4)-OH] for condensation on solid-phase or
Boc-His(Trt)¹-Aib-Asp(OtBu)-Gly⁴-OH [(Boc-Aib²Ted(1-4)-OH] for condensation on solid-phase or
Boc-His(Trt)¹-Gly-Asp(OtBu)-Gly⁴-O-Pfp [(Boc-Ted(1-4)-O-Pfp] for condensation on liquid-phase or
Boc-His(Trt)¹-Aib-Asp(OtBu)-Gly⁴-O-Pfp [(Boc-Aib²Ted(1-4)-O-Pfp] for condensation in liquid-phase
was coupled on solid-phase (R=2-chlorotrityl resin) or in solution (R=H) with one of the following fragments:
H-Ser(tBu)⁵-Phe-Ser(tBu)-Asp(OtBu)-Glu(OtBu)-Met-Asn(Trt)-Thr(tBu)-Ile-Leu¹⁴-O—R [H-Ted(5-14)-O—R]
H-Ser(tBu)⁵-Phe-Ser(tBu)-Asp(OtBu)-Glu(OtBu)-Met-Asn(Trt)-Thr(tBu)-Ile-Leu-Asp(OtBu)-Asn(Trt)-Leu¹⁷-O—R [H-Ted(5-17)-O—R]
H-Ser(tBu)⁵-Phe-Ser(tBu)-Asp(OtBu)-Glu(OtBu)-Met-Asn(Trt)-Thr(tBu)-Ile-Leu-Asp(OtBu)-Asn(Trt)-Leu-Ala¹⁸-O—R [H-Ted(5-18)-O—R]
H-Ser(tBu)⁵-Phe-Ser(tBu)-Asp(OtBu)-Glu(OtBu)-Met-Asn(Trt)-Thr(tBu)-Ile-Leu-Asp(OtBu)-Asn(Trt)-Leu-Ala-Ala¹⁹-O—R [H-Ted(5-19)-O—R]
to give a first fragment of Teduglutide (or its Aib²-analogue) as set out below, after cleavage from the protected peptide from the resin, or directly:
Boc-His(Trt)¹-Gly-Asp(tBu)-Gly-Ser(tBu)-Phe-Ser(tBu)-Asp(tBu)-Glu(tBu)-Met-Asn(Trt)-Thr(tBu)-Ile-Leu¹⁴-OH [Boc-Ted(1-14)-OH]
Boc-His(Trt)¹-Gly-Asp(tBu)-Gly-Ser(tBu)-Phe-Ser(tBu)-Asp(tBu)-Glu(tBu)-Met-Asn(Trt)-Thr(tBu)-Ile-Leu-Asp(tBu)-Asn (Trt)-Leu¹⁷-OH [Boc-Ted(1-17)-OH]
Boc-His(Trt)¹-Gly-Asp(tBu)-Gly-Ser(tBu)-Phe-Ser(tBu)-Asp(tBu)-Glu(tBu)-Met-Asn(Trt)-Thr(tBu)-Ile-Leu-Asp(tBu)-Asn(Trt)-Leu-Ala¹⁸-OH [Boc-Ted(1-18)-OH]
Boc-His(Trt)¹-Gly-Asp(tBu)-Gly-Ser(tBu)-Phe-Ser(tBu)-Asp(tBu)-Glu(tBu)-Met-Asn(Trt)-Thr(tBu)-Ile-Leu-Asp(tBu)-Asn(Trt)-Leu-Ala-Ala¹⁹-OH [Boc-Ted(1-19)-OH]
Boc-His(Trt)¹-Aib-Asp(tBu)-Gly⁴-Ser(tBu)-Phe-Ser(tBu)-Asp(tBu)-Glu(tBu)-Met-Asn(Trt)-Thr(tBu)-Ile-Leu¹⁴-OH [(Boc-Aib²Ted(1-14)-OH]
Boc-His(Trt)¹-Aib-Asp(tBu)-Gly⁴-Ser(tBu)-Phe-Ser(tBu)-Asp(tBu)-Glu(tBu)-Met-Asn(Trt)-Thr(tBu)-Ile-Leu-Asp(tBu)-Asn(Trt)-Leu¹⁷-OH [(Boc-Aib²Ted(1-17)-OH]
Boc-His(Trt)¹-Aib-Asp(tBu)-Gly⁴-Ser(tBu)-Phe-Ser(tBu)-Asp(tBu)-Glu(tBu)-Met-Asn(Trt)-Thr(tBu)-Ile-Leu-Asp(tBu)-Asn(Trt)-Leu-Ala¹⁸-OH [(Boc-Aib²Ted(1-18)-OH]
Boc-His(Trt)¹-Aib-Asp(tBu)-Gly⁴-Ser(tBu)-Phe-Ser(tBu)-Asp(tBu)-Glu(tBu)-Met-Asn(Trt)-Thr(tBu)-Ile-Leu-Asp(tBu)-Asn(Trt)-Leu-Ala-Ala¹⁹-OH [(Boc-Aib²Ted(1-19)-OH]
(where Phe-Ser(tBu)- can also be -Phe-ψSer-).

The first fragment of Teduglutide obtained above was then coupled with the corresponding second fragment selected from:
H-Asp(tBu)¹⁵-Asn(Trt)-Leu-Ala-Ala-Arg(Pbf)-Asp(tBu)-Phe-Ile-Asn(Trt)-Trp(Boc)-Leu-Ile-Gln(Trt)-Thr(tBu)-Lys(Boc)-Ile-Thr(tBu)-Asp(tBu)³³-O—R [(H-Ted(15-33)-O—R],
wherein R=H, Clt, Dpm, tBu as described for Liraglutide and Semaglutide
H-Ala¹⁸-Ala-Arg(Pbf)-Asp(tBu)-Phe-Ile-Asn(Trt)-Trp(Boc)-Leu-Ile-Gln(Trt)-Thr(tBu)-Lys(Boc)-Ile-Thr(tBu)-Asp(tBu)³³-O—R [(H-Ted(18-33)-O—R]
H-Ala¹⁹-Arg(Pbf)-Asp(tBu)-Phe-Ile-Asn(Trt)-Trp(Boc)-Leu-Ile-Gln(Trt)-Thr(tBu)-Lys(Boc)-Ile-Thr(tBu)-Asp(tBu)³³-O—R [(H-Ted(19-33)-O—R]
H-Arg(Pbf)²⁰-Asp(tBu)-Phe-Ile-Asn(Trt)-Trp(Boc)-Leu-Ile-Gln(Trt)-Thr(tBu)-Lys(Boc)-Ile-Thr(tBu)-Asp(tBu)³³-O—R [(H-Ted(20-33)-O—R]
where R=2-chlorotrityl resin or H, and where -Gln(Trt)-Thr(tBu)- can also be -Gln(Trt)-ψThr-
to form Teduglutide (1-33), after the cleavage from the resin and deprotection, or direct deprotection. See Table 2 for results.

Various modifications and variations of the described aspects of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the relevant fields are intended to be within the scope of the following claims.

TABLE 1
Racemization and yield determined during the fragment condensation synthesis of Liraglutide and Semaglutide under various
conditions. Scale: 0.01 mmol; Ratio: Fragment 1/Fragment 2 (1/1.05).

| Run | | | Condensing | | Temp | | Yield |
|---|---|---|---|---|---|---|---|
| No. | Fragment 1 | Fragment 2 | agent | Solvent | [° C.] | %-D¹⁾ | [%]²⁾ |
| 1 | Boc-Lir(1-19)-OH; [SEQ ID NO: 5] | [SEQ ID NO: 6]; P3 = Wang resin | DIC/HOBt | NMP | 22 | 37.4* | 13.4 |
| 2 | Boc-Lir(1-19)-OH; [SEQ ID NO: 5] | [SEQ ID NO: 6]; P3 = CTC resin | DIC/HOBt | NMP | 22 | 35.6* | 16.8 |
| 3 | Boc-Lir(1-19)-OH; [SEQ ID NO: 5] | [SEQ ID NO: 6]; P3 = H | DIC/HOBt | NMP | 22 | 5.4 | 27.4 |
| 4 | Boc-Lir(1-19)-OH; [SEQ ID NO: 59] | [SEQ ID NO: 6]; P3 = H | DIC/HOBt | NMP | 22 | 6.1 | 22.7 |
| 5 | Boc-Lir(1-19)-OH; [SEQ ID NO: 5] | [SEQ ID NO: 6]; P3 = H | DIC/HOBt | NMP | 10 | 3.4 | 22.1 |
| 6 | Boc-Lir(1-19)-OH; [SEQ ID NO: 59] | [SEQ ID NO: 6]; P3 = H | DIC/HOBt | NMP | 10 | 3.7 | 25.8 |
| 7 | Boc-Lir(1-19)-OH; [SEQ ID NO: 5] | [SEQ ID NO: 6]; P3 = Clt | DIC/HOBt | NMP | 22 | 4.6 | 34.5 |
| 8 | Boc-Lir(1-19)-OH; [SEQ ID NO: 59] | [SEQ ID NO: 6]; P3 = Clt | DIC/HOBt | NMP | 22 | 4.2 | 36.7 |
| 9 | Boc-Lir(1-19)-OH; [SEQ ID NO: 5] | [SEQ ID NO: 6]; P3 = Clt | DIC/HOBt | NMP | 10 | 3.3 | 35.6 |
| 10 | Boc-Lir(1-19)-OH; [SEQ ID NO: 59] | [SEQ ID NO: 6]; P3 = Clt | DIC/HOBt | NMP | 10 | 3.5 | 37.2 |
| 11 | Boc-Lir(1-19)-OH; [SEQ ID NO: 5] | [SEQ ID NO: 6]; P3 = Dpm | DIC/HOBt | NMP | 22 | 4.5 | 32.5 |
| 12 | Boc-Lir(1-19)-OH; [SEQ ID NO: 59] | [SEQ ID NO: 6]; P3 = Dpm | DIC/HOBt | NMP | 22 | 4.4 | 33.5 |
| 13 | Boc-Lir(1-19)-OH; [SEQ ID NO: 5] | [SEQ ID NO: 6]; P3 = Dpm | DIC/HOBt | NMP | 10 | 3.1 | 36.4 |
| 14 | Boc-Lir(1-19)-OH; [SEQ ID NO: 59] | [SEQ ID NO: 6]; P3 = Dpm | DIC/HOBt | NMP | 10 | 2.9 | 37.7 |
| 15 | Boc-Lir(1-19)-OH; [SEQ ID NO: 5] | [SEQ ID NO: 6]; P3 = tBu | DIC/HOBt | NMP | 22 | 3.9 | 54.5 |
| 16 | Boc-Lir(1-19)-OH; [SEQ ID NO: 59] | [SEQ ID NO: 6]; P3 = tBu | DIC/HOBt | NMP | 22 | 4.6 | 52.2 |
| 17 | Boc-Lir(1-19)-OH; [SEQ ID NO: 5] | [SEQ ID NO: 6]; P3 = tBu | DIC/HOBt | NMP | 10 | 3.3 | 77.7 |
| 18 | Boc-Lir(1-19)-OH; [SEQ ID NO: 59] | [SEQ ID NO: 6]; P3 = tBu | DIC/HOBt | NMP | 10 | 3.0 | 75.4 |
| 19 | Boc-Lir(1-19)-OH; [SEQ ID NO: 5] | [SEQ ID NO: 6]; P3 = tBu | DIC/HOBt | NMP | 10 | 3.3 | 76.3 |
| 20 | Boc-Lir(1-19)-OH; [SEQ ID NO: 59] | [SEQ ID NO: 6]; P3 = tBu | DIC/HOBt | NMP | 2-5 | 2.1 | 81.7 |
| 21 | Boc-Lir(1-19)-OH; [SEQ ID NO: 5] | [SEQ ID NO: 6]; P3 = tBu | DIC/HOBt | DMF | 2-5 | 2.4 | 82.2 |
| 22 | Boc-Lir(1-19)-OH; [SEQ ID NO: 59] | [SEQ ID NO: 6]; P3 = tBu | DIC/HOBt | DMF | 2-5 | 2.3 | 83.1 |
| 23 | Boc-Lir(1-19)-OH; [SEQ ID NO: 5] | [SEQ ID NO: 6]; P3 = tBu | DIC/HOBt | DMF/DCM | 2-5 | 2.7 | 76.4 |
| | | | | (1:1) | | | |
| 24 | Boc-Lir(1-19)-OH; [SEQ ID NO: 59] | [SEQ ID NO: 6]; P3 = tBu | DIC/HOBt | DMF/DCM | 2-5 | 2.6 | 77.7 |
| | | | | (1:1) | | | |
| 25 | Boc-Lir(1-19)-OH; [SEQ ID NO: 5] | [SEQ ID NO: 6]; P3 = tBu | DIC/HOBt | DCM | 2-5 | 2.8 | 62.4 |
| 26 | Boc-Lir(1-19)-OH; [SEQ ID NO: 59] | [SEQ ID NO: 6]; P3 = tBu | DIC/HOBt | DCM | 2-5 | 2.2 | 66.4 |
| 27 | Boc-Lir(1-19)-OH; [SEQ ID NO: 59] | [SEQ ID NO: 6]; P3 = tBu | EDAC/HOBt/ | DMF | 2-5 | 1.9 | 84.7 |
| | | | DIPEA | | | | |
| 28 | Boc-Lir(1-19)-OH; [SEQ ID NO: 59] | [SEQ ID NO: 6]; P3 = tBu | HBTU/HOBt/ | DMF | 2-5 | 4.9 | 83.2 |
| | | | DIPEA | | | | |
| 29 | Boc-Lir(1-19)-OSu [SEQ ID NO: 4]; | [SEQ ID NO: 6]; P3 = tBu | EDAC/HOBt/ | DMF | 2-5 | 13.7 | 34.4 |
| | P2 = Su | | DIPEA | | | | |
| 30 | Boc-Lir(1-19)-OSu [SEQ ID NO: 4]; | [SEQ ID NO: 6]; P3 = tBu | EDAC/HOBt/ | DMF | 2-5 | 14.4 | 29.2 |
| | P2 = Pfp | | DIPEA | | | | |
| 32 | Boc-Sem(1-19)-OH [SEQ ID NO: 12] | [SEQ ID NO: 13]; P3 = tBu | EDAC/HOBt/ | DMF | 2-5 | 2.4 | 81.3 |
| | | | DIPEA | | | | |
| 33 | Boc-Sem(1-19)-OH [SEQ ID NO: 60] | [SEQ ID NO: 13]; P3 = tBu | EDAC/HOBt/ | DMF | 2-5 | 2.1 | 82.7 |
| | ΨSer | | DIPEA | | | | |
| 34 | Boc-Lir(1-18)-OH [SEQ ID NO: 18] | [SEQ ID NO: 20] H-Lir(19-31)-O-Clt | EDAC/HOBt/ | DMF | 2-5 | 2.9 | 67.6 |
| | | | DIPEA | | | | |
| 35 | Boc-Lir(1-17)-OH [SEQ ID NO: 30] | [SEQ ID NO: 32] H-Lir(18-31)-O-Clt | EDAC/HOBt/ | DMF | 2-5 | 2.6 | 44.4 |
| | | | DIPEA | | | | |
| 36 | Boc-Lir(1-14)-OH [SEQ ID NO: 42] | H-Lir(15-31)-O-Clt [SEQ ID NO: 44] | EDAC/HOBt/ | DMF | 2-5 | 2.7 | 24.3 |
| | | | DIPEA | | | | |

¹⁾The percentage of D-peptide was determined by HPLC setting the area of the peaks of L-peptide + D-peptide = 100%.
²⁾The yield percentage was determined by seting the total peaks area = 100% and comparing with area of the peak of the D-peptide.
*The condensation could not be completed even by applying a five molar excess of Fragment 1.

TABLE 2
Racemization and yield determined during the fragment condensation synthesis of Teduglutide under
various conditions. Scale: 0.01 mmol; Ratio: Fragment 1/Fragment 2 (1/1.05).

| Run | | | Condensing | | Temp | | Yield |
|---|---|---|---|---|---|---|---|
| Nr. | Fragment 1 | Fragment 2 | agent | Solvent | [° C.] | %-D¹⁾ | [%]²⁾ |
| 1 | [Boc-Ted(1-14)-OH] | H-Ted(15-33)-O-R | DIC/HOBt | DMF | 2-5 | 28.3 | 26.5 |
| | | R = 2-chlorotrityl | | | | | |
| 2 | [Boc-Ted(1-14)-OH] | H-Ted(15-33)-O-R | DIC/HOBt | DMF | 2-5 | 3.2 | 58.2 |
| | | R = H | | | | | |
| 3 | Boc-Ted(1-17)-OH | H-Ted(18-33)-O-R | DIC/HOBt | DMF | 2-5 | 24.7 | 23.5 |
| | | R = 2-chlorotrityl | | | | | |
| 4 | Boc-Ted(1-17)-OH | H-Ted(18-33)-O-R | DIC/HOBt | DMF | 2-5 | 3.4 | 55.5 |
| | | R = H | | | | | |
| 5 | Boc-Ted(1-18)-OH | H-Ted(19-33)-O-R | DIC/HOBt | DMF | 2-5 | 26.2 | 24.8 |
| | | R = 2-chlorotrityl | | | | | |
| 6 | Boc-Ted(1-18)-OH | H-Ted(19-33)-O-R | DIC/HOBt | DMF | 2-5 | 2.9 | 62.2 |
| | | R = H | | | | | |
| 7 | Boc-Ted(1-19)-OH | H-Ted(20-33)-O-R | DIC/HOBt | DMF | 2-5 | 25.6 | 18.7 |
| | | R = 2-chlorotrityl | | | | | |
| 8 | Boc-Ted(1-19)-OH | H-Ted(20-33)-O-R | DIC/HOBt | DMF | 2-5 | 3.1 | 59.4 |
| | | R = H | | | | | |
| 9 | [Boc-Ted(1-14)-OH] | H-Ted(15-33)-O-R | HBTU/DIPEA/HOBt | DMF | 2-5 | 19.4 | 27.2 |
| | | R = 2-chlorotrityl | | | | | |
| 10 | [Boc-Ted(1-14)-OH] | H-Ted(15-33)-O-R | HBTU/DIPEA/HOBt | DMF | 2-5 | 1.8 | 65.7 |
| | | R = H | | | | | |
| 11 | Boc-Ted(1-17)-OH | H-Ted(18-33)-O-R | HBTU/DIPEA/HOBt | DMF | 2-5 | 2.2 | 64.2 |
| | | R = H | | | | | |
| 12 | Boc-Ted(1-18)-OH | H-Ted(19-33)-O-R | HBTU/DIPEA/HOBt | DMF | 2-5 | 1.9 | 59.9 |
| | | R = H | | | | | |
| 13 | Boc-Ted(1-19)-OH | H-Ted(20-33)-O-R | HBTU/DIPEA/HOBt | DMF | 2-5 | 2.4 | 66.3 |
| | | R = H | | | | | |
| 14 | Boc-Aib2Ted(1-18)-OH | H-Ted(19-33)-O-R | HBTU/DIPEA/HOBt | DMF | 2-5 | 2.4 | 62.7 |
| | | R = H | | | | | |
| 15 | Boc-Ted(1-18)-OH | H-Ted(19-33)-O-R | DIC/HOBt | DMF | 2-5 | 2.8 | 62.2 |
| | | R = Clt | | | | | |
| 16 | Boc-Ted(1-18)-OH | H-Ted(19-33)-O-R | DIC/HOBt | DMF | 2-5 | 2.9 | 57.5 |
| | | R = Dpm | | | | | |
| 17 | Boc-Ted(1-18)-OH | H-Ted(19-33)-O-R | DIC/HOBt | DMF | 2-5 | 2.2 | 68.2 |
| | | R = tBu | | | | | |
| 18 | Boc-Ted(1-18)-OH | H-Ted(19-33)-O-R | DIC/HOBt | DMF | 2-5 | 2.4 | 69.9 |
| | | R = tBu; Ser5 = ΨSer | | | | | |
| 19 | Boc-Aib2Ted(1-18)-OH | H-Ted(19-33)-O-R | DIC/HOBt | DMF | 2-5 | 2.4 | 59.8 |
| | | R = H; Ser5 = ΨSer | | | | | |

1)The percentage of D-peptide was determined by HPLC setting the area of the peaks of L-peptide + D-peptide = 100%.
²⁾The yield percentage was determined by seting the total peaks area = 100% and comparing with area of the peak of the D-peptide.

1. A process for preparing a glucagon-like peptide (GLP), or an analogue or variant thereof, said process comprising coupling in solution at least a first fragment and at least a second fragment, wherein the coupling comprises reacting the carboxy terminal amino acid of the first fragment with the amino terminal amino acid of the second fragment, and wherein the carboxy terminal amino acid of the first fragment is other than a Gly residue.

2-8. (canceled)

9. The process according to claim 1 wherein the GLP peptide or analogue or variant thereof is of SEQ ID NO: 1:
1 2 3 4 5 6 7 8 9 10
His-Ala-Glu-Gly-Thr-Phe-Thr-Ser-Asp-Val-
11 12 13 14 15 16 17 18 19
Ser-Ser-Tyr-Leu-Glu-Gly-Gln-Ala-Ala-
20 21 22 23 24 25 26 27
Lys(Pal-Glu)-Glu-Phe-Ile-Ala-Trp-Leu-Val-
28 29 30 31
Arg-Gly-Arg-Gly-OH
and said process comprises:
(i) coupling a first peptide having the sequence:
[SEQ ID NO: 2]
His-Ala-Glu-Gly-Thr-Phe-Thr-Ser-Asp-Val-
Ser-Ser-Tyr-Leu-Glu-Gly-Gln-Ala-Ala
wherein:
the N-terminal of His is optionally protected with a protecting group; and
the Ala carboxylic acid group is optionally in the form of an activated carboxylic acid derivative;
in solution with a second peptide having the sequence:
[SEQ ID NO: 3]
Lys(Pal-Glu-OX)-Glu-Phe-Ile-Ala-Trp-Leu-Val-Arg-
Gly-Arg-Gly-OH
wherein:
X is H or a protecting group for the Glu carboxylic acid group;
and wherein one or more of the amino acid residues in said first and second peptides may be unprotected or protected;
(ii) optionally removing any protecting groups;
(iii) optionally purifying the GLP peptide.

10. The process according to claim 9 wherein the first peptide has the formula:
[SEQ ID NO: 4]
P1-His(P)-Ala-Glu(P)-Gly-Thr(P)-Phe-Thr(P)-
Ser(P)-Asp(P)-Val-Ser(P)-Ser(P)-Tyr(P)-Leu-
Glu(P)-Gly-Gln(P)-Ala-Ala-O-P2
wherein:
P1 is a protecting group for the N-terminal of His;
each P represents a side chain protecting group which may be the same or different; and
P2 is H or an activated carboxylic ester of the Ala residue.

11-14. (canceled)

15. The process according to claim 1 wherein the GLP peptide or analogue or variant thereof is of SEQ ID NO: 8:
1 2 3 4 5 6 7 8 9 10 11 12
His-Aib-Glu-Gly-Thr-Phe-Thr-Ser-Asp-Val-Ser-Ser-
13 14 15 16 17 18 19 20 21
Tyr-Leu-Glu-Gly-Gln-Ala-Ala-Lys(W)-Glu-Phe-
22 23 24 25 26 27 28 29 30 31
Ile-Ala-Trp-Leu-Val-Arg-Gly-Arg-Gly-OH
where W is N-(17-carboxy-1-oxoheptadecyl)-L-y-glutamyl-2-[2-(2-aminoethoxy) ethoxy] acetyl-2-[2-(2-aminoethoxy)ethoxy]acetyl;
and said process comprises:
(i) coupling a first peptide having the sequence:
[SEQ ID NO: 9]
His-Aib-Glu-Gly-Thr-Phe-Thr-Ser-Asp-Val-Ser-Ser-
Tyr-Leu-Glu-Gly-Gln-Ala-Ala
wherein:
the N-terminal of His is optionally protected with a protecting group; and
the Ala carboxylic acid group is optionally in the form of an activated carboxylic acid derivative;
in solution with a second peptide having the sequence:
[SEQ ID NO: 10]
Lys(W1)-Glu-Phe-Ile-Ala-Trp-Leu-Val-Arg-Gly-Arg-
Gly-OH
wherein:
W1 is N-(17-carboxy-1-oxoheptadecyl)-L-γ-glutamyl-2-[2-(2-aminoethoxy) ethoxy]acetyl-2-[2-(2-aminoethoxy)ethoxy]acetyl;
and wherein one or more of the amino acid residues in said first and second peptides and W1 may be unprotected or protected;
(ii) optionally removing any protecting groups;
(iii) optionally purifying the GLP peptide.

16. The process according to claim 15 wherein the first peptide has the formula:
[SEQ ID NO: 11]
P1-His(P)-Aib-Glu(P)-Gly-Thr(P)-Phe-Thr(P)-
Ser(P)-Asp(P)-Val-Ser(P)-Ser(P)-Tyr(P)-
Leu-Glu(P)-Gly-Gln(P)-Ala-Ala-O-P2
wherein:
P1 is a protecting group for the N-terminal of His;
each P represents a side chain protecting group which may be the same or different; and
P2 is H or an activated carboxylic ester of the Ala residue.

17-19. (canceled)

20. The process according to claim 1 wherein the GLP peptide or analogue or variant thereof is of SEQ ID NO: 1:
1 2 3 4 5 6 7 8 9 10
His-Ala-Glu-Gly-Thr-Phe-Thr-Ser-Asp-Val-
11 12 13 14 15 16 17 18 19
Ser-Ser-Tyr-Leu-Glu-Gly-Gln-Ala-Ala-
20 21 22 23 24 25 26 27
Lys(Pal-Glu)-Glu-Phe-Ile-Ala-Trp-Leu-Val-
28 29 30 31
Arg-Gly-Arg-Gly-OH
and said process comprises:
(i) coupling a first peptide having the sequence:
[SEQ ID NO: 15]
His-Ala-Glu-Gly-Thr-Phe-Thr-Ser-Asp-Val-Ser-
Ser-Tyr-Leu-Glu-Gly-Gln-Ala
wherein:
the N-terminal of His is optionally protected with a protecting group; and
the Ala carboxylic acid group is optionally in the form of an activated carboxylic acid derivative;
in solution with a second peptide having the sequence:
[SEQ ID NO: 16]
Ala-Lys(Pal-Glu-OX)-Glu-Phe-Ile-Ala-Trp-Leu-
Val-Arg-Gly-Arg-Gly-OH
wherein:
X is H or a protecting group for the Glu carboxylic acid group;
and wherein one or more of the amino acid residues in the first and second peptides may be unprotected or protected;
(ii) optionally removing any protecting groups;
(iii) optionally purifying the GLP peptide.

21. The process according to claim 20 wherein the first peptide has the formula:
[SEQ ID NO: 17]
P1-His(P)-Ala-Glu(P)-Gly-Thr(P)-Phe-Thr(P)-
Ser(P)-Asp(P)-Val-Ser(P)-Ser(P)-Tyr(P)-Leu-
Glu(P)-Gly-Gln(P)-Ala-O-P2
wherein:
P1 is a protecting group for the N-terminal of His;
each P represents a side chain protecting group which may be the same or different; and
P2 is H or an activated carboxylic ester of the Ala residue.

22-24. (canceled)

25. The process according to claim 1 wherein the GLP peptide or analogue or variant thereof is of SEQ ID NO: 8:
1 2 3 4 5 6 7 8 9 10 11 12
His-Aib-Glu-Gly-Thr-Phe-Thr-Ser-Asp-Val-Ser-Ser-
13 14 15 16 17 18 19 20 21
Tyr-Leu-Glu-Gly-Gln-Ala-Ala-Lys(W)-Glu-Phe-
22 23 24 25 26 27 28 29 30 31
Ile-Ala-Trp-Leu-Val-Arg-Gly-Arg-Gly-OH
where W is N-(17-carboxy-1-oxoheptadecyl)-L-γ-glutamyl-2-[2-(2-aminoethoxy) ethoxy]acetyl-2-[2-(2-aminoethoxy)ethoxy]acetyl;
and said process comprises:
(i) coupling a first peptide having the sequence:
[SEQ ID NO: 21]
His-Aib-Glu-Gly-Thr-Phe-Thr-Ser-Asp-Val-Ser-Ser-
Tyr-Leu-Glu-Gly-Gln-Ala
wherein:
the N-terminal of His is optionally protected with a protecting group; and
the Ala carboxylic acid group is optionally in the form of an activated carboxylic acid derivative;
in solution with a second peptide having the sequence:
[SEQ ID NO: 22]
Ala-Lys(W1)-Glu-Phe-Ile-Ala-Trp-Leu-Val-Arg-Gly-
Arg-Gly-OH
wherein:
W1 is N-(17-carboxy-1-oxoheptadecyl)-L-γ-glutamyl-2-[2-(2-aminoethoxy) ethoxy]acetyl-2-[2-(2-aminoethoxy)ethoxy]acetyl;
and wherein one or more of the amino acid residues in said first and second peptides and W1 may be unprotected or protected;
(ii) optionally removing any protecting groups;
(iii) optionally purifying the GLP peptide.

26. The process according to claim 25 wherein the first peptide has the formula:
[SEQ ID NO: 23]
P1-His(P)-Aib-Glu(P)-Gly-Thr(P)-Phe-Thr(P)-Ser(P)-
Asp(P)-Val-Ser(P)-Ser(P)-Tyr(P)-Leu-Glu(P)-Gly-
Gln(P)-Ala-O-P2
wherein:
P1 is a protecting group for the N-terminal of His;
each P represents a side chain protecting group which may be the same or different; and
P2 is H or an activated carboxylic ester of the Ala residue.

27-29. (canceled)

30. The process according to claim 1 wherein the GLP peptide or analogue or variant thereof is of SEQ ID NO: 1:
1 2 3 4 5 6 7 8 9 10 11 12
His-Ala-Glu-Gly-Thr-Phe-Thr-Ser-Asp-Val-Ser-Ser-
13 14 15 16 17 18 19 20 21
Tyr-Leu-Glu-Gly-Gln-Ala-Ala-Lys(Pal-Glu)-Glu-
22 23 24 25 26 27 28 29 30 31
Phe-Ile-Ala-Trp-Leu-Val-Arg-Gly-Arg-Gly-OH
and said process comprises:
(i) coupling a first peptide having the sequence:
[SEQ ID NO: 27]
His-Ala-Glu-Gly-Thr-Phe-Thr-Ser-Asp-Val-Ser-Ser-
Tyr-Leu-Glu-Gly-Gln
wherein:
the N-terminal of His is optionally protected with a protecting group; and
the Gln carboxylic acid group is optionally in the form of an activated carboxylic acid derivative;
in solution with a second peptide having the sequence:
[SEQ ID NO: 28]
Ala-Ala-Lys(Pal-Glu-OX)-Glu-Ile-Ala-Trp-Leu-Val-
Arg-Gly-Arg-Gly-OH
wherein:
X is H or a protecting group for the Glu carboxylic acid group;
and wherein one or more of the amino acid residues in the first and second peptides may be unprotected or protected;
(ii) optionally removing any protecting groups;
(iii) optionally purifying the GLP peptide.

31. The process according to claim 30 wherein the first peptide has the formula:
[SEQ ID NO: 29]
P1-His(P)-Ala-Glu(P)-Gly-Thr(P)-Phe-Thr(P)-Ser(P)-
Asp(P)-Val-Ser(P)-Ser(P)-Tyr(P)-Leu-Glu(P)-Gly-Gln-
O-P2
wherein:
P1 is a protecting group for the N-terminal of His;
each P represents a side chain protecting group which may be the same or different; and
P2 is H or an activated carboxylic ester of the Gln residue.

32-34. (canceled)

35. The process according to claim 1 wherein the GLP peptide or analogue or variant thereof is of SEQ ID NO: 8:
1 2 3 4 5 6 7 8 9 10 11 12
His-Aib-Glu-Gly-Thr-Phe-Thr-Ser-Asp-Val-Ser-Ser-
13 14 15 16 17 18 19 20 21 22
Tyr-Leu-Glu-Gly-Gln-Ala-Ala-Lys(W)-Glu-Phe-
23 24 25 26 27 28 29 30 31
Ile-Ala-Trp-Leu-Val-Arg-Gly-Arg-Gly-OH
where W is N-(17-carboxy-1-oxoheptadecyl)-L-γ-glutamyl-2-[2-(2-aminoethoxy)ethoxy]acetyl-2-[2-(2-aminoethoxy)ethoxy]acetyl;
and said process comprises:
(i) coupling a first peptide having the sequence:
[SEQ ID NO: 33]
His-Aib-Glu-Gly-Thr-Phe-Thr-Ser-Asp-Val-Ser-Ser-
Tyr-Leu-Glu-Gly-Gln
wherein:
the N-terminal of His is optionally protected with a protecting group; and
the Gln carboxylic acid group is optionally in the form of an activated carboxylic acid derivative;
in solution with a second peptide having the sequence:
[SEQ ID NO: 34]
Ala-Ala-Lys(W1)-Glu-Phe-Ile-Ala-Trp-Leu-Val-Arg-
Gly-Arg-Gly-OH
wherein:
W1 is N-(17-carboxy-1-oxoheptadecyl)-L-γ-glutamyl-2-[2-(2-aminoethoxy) ethoxy]acetyl-2-[2-(2-aminoethoxy)ethoxy]acetyl;
and wherein one or more of the amino acid residues in said first and second peptides and W1 may be unprotected or protected;
(ii) optionally removing any protecting groups;
(iii) optionally purifying the GLP peptide.

36. The process according to claim 35 wherein the first peptide has the formula:
[SEQ ID NO: 35]
P1-His(P)-Aib-Glu(P)-Gly-Thr(P)-Phe-Thr(P)-
Ser(P)-Asp(P)-Val-Ser(P)-Ser(P)-Tyr(P)-Leu-
Glu(P)-Gly-Gln-O-P2
wherein:
P1 is a protecting group for the N-terminal of His;
each P represents a side chain protecting group which may be the same or different; and
P2 is H or an activated carboxylic ester of the Gln residue.

37-39. (canceled)

40. The process according to claim 1 wherein the GLP peptide or analogue or variant thereof is of SEQ ID NO: 1:
1 2 3 4 5 6 7 8 9 10 11 12
His-Ala-Glu-Gly-Thr-Phe-Thr-Ser-Asp-Val-Ser-Ser-
13 14 15 16 17 18 19 20 21
Tyr-Leu-Glu-Gly-Gln-Ala-Ala-Lys(Pal-Glu)-Glu-
22 23 24 25 26 27 28 29 30 31
Phe-Ile-Ala-Trp-Leu-Val-Arg-Gly-Arg-Gly-OH
and said process comprises:
(i) coupling a first peptide having the sequence:
[SEQ ID NO: 39]
His-Ala-Glu-Gly-Thr-Phe-Thr-Ser-Asp-Val-Ser-Ser-
Tyr-Leu
wherein:
the N-terminal of His is optionally protected with a protecting group; and
the Leu carboxylic acid group is optionally in the form of an activated carboxylic acid derivative;
in solution with a second peptide having the sequence:
[SEQ ID NO: 40]
Glu-Gly-Gln-Ala-Ala-Lys(Pal-Glu-OX)-Glu-Phe-Ile-
Ala-Trp-Leu-Val-Arg-Gly-Arg-Gly-OH
wherein:
X is H or a protecting group for the Glu carboxylic acid group;
and wherein one or more of the amino acid residues in said first and second peptides may be unprotected or protected;
(ii) optionally removing any protecting groups;
(iii) optionally purifying the GLP peptide.

41. The process according to claim 40 wherein the first peptide has the formula:
[SEQ ID NO: 41]
P1-His(P)-Ala-Glu(P)-Gly-Thr(P)-Phe-Thr(P)-
Ser(P)-Asp(P)-Val-Ser(P)-Ser(P)-Tyr(P)-
Leu-O-P2
wherein:
P1 is a protecting group for the N-terminal of His;
each P represents a side chain protecting group which may be the same or different; and
P2 is H or an activated carboxylic ester of the Leu residue.

42-44. (canceled)

45. The process according to claim 1 wherein the GLP peptide or analogue or variant thereof is of SEQ ID NO: 8:
1 2 3 4 5 6 7 8 9 10 11 12
His-Aib-Glu-Gly-Thr-Phe-Thr-Ser-Asp-Val-Ser-Ser-
13 14 15 16 17 18 19 20 21 22
Tyr-Leu-Glu-Gly-Gln-Ala-Ala-Lys(W)-Glu-Phe-
23 24 25 26 27 28 29 30 31
Ile-Ala-Trp-Leu-Val-Arg-Gly-Arg-Gly-OH
where W is N-(17-carboxy-1-oxoheptadecyl)-L-γ-glutamyl-2-[2-(2-aminoethoxy) ethoxy] acetyl-2-[2-(2-aminoethoxy)ethoxy]acetyl;
and said process comprises:
(i) coupling a first peptide having the sequence:
[SEQ ID NO: 45]
His-Aib-Glu-Gly-Thr-Phe-Thr-Ser-Asp-Val-Ser-Ser-
Tyr-Leu
wherein:
the N-terminal of His is optionally protected with a protecting group, preferably Boc, or Fmoc; and
the Leu carboxylic acid group is optionally in the form of an activated carboxylic acid derivative;
in solution with a second peptide having the sequence:
[SEQ ID NO: 46]
Glu-Gly-Gln-Ala-Ala-Lys(W1)-Glu-Phe-Ile-Ala-Trp-
Leu-Val-Arg-Gly-Arg-Gly-OH
wherein:
W1 is N-(17-carboxy-1-oxoheptadecyl)-L-γ-glutamyl-2-[2-(2-aminoethoxy) ethoxy]acetyl-2-[2-(2-aminoethoxy)ethoxy]acetyl;
and wherein one or more of the amino acid residues in said first and second peptides and W1 may be unprotected or protected;
(ii) optionally removing any protecting groups;
(iii) optionally purifying the GLP peptide.

46. The process according to claim 45 wherein the first peptide has the formula:
[SEQ ID NO: 47]
P1-His(P)-Aib-Glu(P)-Gly-Thr(P)-Phe-Thr(P)-
Ser(P)-Asp(P)-Val-Ser(P)-Ser(P)-Tyr(P)-Leu-
O-P2
wherein:
P1 is a protecting group for the N-terminal of His;
each P represents a side chain protecting group which may be the same or different; and
P2 is H or an activated carboxylic ester of the Leu residue.

47-49. (canceled)

50. The process for preparing a GLP peptide of SEQ ID NO: 1 according to claim 9, wherein the second peptide is prepared by solid phase synthesis using Lys(Pal-Glu-O^{t}Bu)-OH.

51. (canceled)

52. The process for preparing a GLP peptide of SEQ ID NO: 8 according to claim 15, wherein the second peptide is prepared by solid phase synthesis using Lys(C18-Glu-PEG2)-OH.

53. (canceled)

54. The method according to claim 9 wherein the peptide of [SEQ ID NO: 2] is prepared from His-Ala-Glu-Gly [SEQ ID NO: 51] by stepwise solid phase synthesis.

55-89. (canceled)


SEQUENCE LISTING (165 sequences)

SEQ ID NO: 1 - protein, 31 aa, synthetic construct
  REGION 1..31: note = Glucagon-like peptide (GLP-1) peptide
  MOD_RES 20: note = N6-(4-carboxy-4-palmitamidobutanoyl)lysine
HAEGTFTSDVSSYLEGQAAKEFIAWLVRGRG

SEQ ID NO: 2 - protein, 19 aa, synthetic construct
  REGION 1..19: note = synthetic peptide
HAEGTFTSDVSSYLEGQAA

SEQ ID NO: 3 - protein, 12 aa, synthetic construct
  REGION 1..12: note = synthetic peptide
  MOD_RES 1: note = N6-(4-carboxy-4-palmitamidobutanoyl)lysine or O-protected N6-(4-carboxy-4-palmitamidobutanoyl)lysine
  VARIANT 1: note = N6-(4-carboxy-4-palmitamidobutanoyl)lysine or O-protected N6-(4-carboxy-4-palmitamidobutanoyl)lysine
KEFIAWLVRGRG

SEQ ID NO: 4 - protein, 19 aa, synthetic construct
  REGION 1..19: note = synthetic peptide
  MOD_RES 1: note = His has N-terminal and side chain protecting groups
  MOD_RES 3: note = Glu has side chain protecting group
  MOD_RES 5: note = Thr has side chain protecting group
  MOD_RES 7: note = Thr has side chain protecting group
  MOD_RES 8: note = Ser has side chain protecting group
  MOD_RES 9: note = Asp has side chain protecting group
  MOD_RES 11: note = Ser has side chain protecting group
  MOD_RES 12: note = Ser has side chain protecting group
  MOD_RES 13: note = Tyr has side chain protecting group
  MOD_RES 15: note = Glu has side chain protecting group
  MOD_RES 17: note = Gln has side chain protecting group
  MOD_RES 19: note = Ala-OH or activated carboxylic ester of Ala
  VARIANT 19: note = Ala-OH or activated carboxylic ester of Ala
HAEGTFTSDVSSYLEGQAA

SEQ ID NO: 5 - protein, 19 aa, synthetic construct
  REGION 1..19: note = synthetic peptide
  MOD_RES 1: note = N-alpha-(tert-butoxycarbonyl)-N-tau-trityl-L-histid ine
  MOD_RES 3: note = (S)-2-amino-5-(tert-butoxy)-5-oxopentanoic acid
  MOD_RES 5: note = O-(tert-butyl)-L-threonine
  MOD_RES 7: note = O-(tert-butyl)-L-threonine
  MOD_RES 8: note = O-(tert-butyl)-L-serine
  MOD_RES 9: note = (S)-2-amino-4-(tert-butoxy)-4-oxobutanoic acid
  MOD_RES 11: note = O-(tert-butyl)-L-serine
  MOD_RES 12: note = O-(tert-butyl)-L-serine
  MOD_RES 13: note = (S)-2-amino-3-(4-(tert-butoxy)phenyl)propanoic acid
  MOD_RES 15: note = (S)-2-amino-5-(tert-butoxy)-5-oxopentanoic acid
  MOD_RES 17: note = N5-trityl-L-glutamine
HAEGTFTSDVSSYLEGQAA

SEQ ID NO: 6 - protein, 12 aa, synthetic construct
  REGION 1..12: note = synthetic peptide
  MOD_RES 1: note = N6-(4-carboxy-4-palmitamidobutanoyl)lysine
  MOD_RES 2: note = Glu has side chain protecting group
  MOD_RES 6: note = Trp has side chain protecting group
  MOD_RES 9: note = Arg has side chain protecting group
  MOD_RES 11: note = Arg has side chain protecting group
  MOD_RES 12: note = Gly-OH or a carboxy protecting group
  VARIANT 12: note = Gly-OH or a carboxy protecting group
KEFIAWLVRGRG

SEQ ID NO: 7 - protein, 12 aa, synthetic construct
  REGION 1..12: note = synthetic peptide
  MOD_RES 1: note = N6-(4-carboxy-4-palmitamidobutanoyl)lysine
  MOD_RES 2: note = (S)-2-amino-5-(tert-butoxy)-5-oxopentanoic acid
  MOD_RES 6: note = 1-(tert-butoxycarbonyl)-L-tryptophan
  MOD_RES 9: note = N-omega-((2,2,4,6,7-pentamethyl-2,3-dihydrobenzofur an-5-yl)sulfonyl)-L-arginine
  MOD_RES 11: note = N-omega-((2,2,4,6,7-pentamethyl-2,3-dihydrobenzofur an-5-yl)sulfonyl)-L-arginine
KEFIAWLVRGRG

SEQ ID NO: 8 - protein, 31 aa, synthetic construct
  REGION 1..31: note = GLP-1 peptide
  MOD_RES 2: note = Aib
  MOD_RES 20: note = Lys(N-(17-carboxy-1-oxoheptadecyl)-L-gamma-glutamyl -2-[2-(2-amino ethoxy)ethoxy]acetyl-2-[2-(2-aminoethoxy)ethoxy]acetyl)
HXEGTFTSDVSSYLEGQAAKEFIAWLVRGRG

SEQ ID NO: 9 - protein, 19 aa, synthetic construct
  REGION 1..19: note = synthetic peptide
  MOD_RES 2: note = Aib
HXEGTFTSDVSSYLEGQAA

SEQ ID NO: 10 - protein, 12 aa, synthetic construct
  REGION 1..12: note = synthetic peptide
  MOD_RES 1: note = Lys(N-(17-carboxy-1-oxoheptadecyl)-L-gamma-glutamyl -2-[2-(2-amino ethoxy)ethoxy]acetyl-2-[2-(2-aminoethoxy)ethoxy]acetyl)
KEFIAWLVRGRG

SEQ ID NO: 11 - protein, 19 aa, synthetic construct
  REGION 1..19: note = synthetic peptide
  MOD_RES 1: note = His has N-terminal and side chain protecting group
  MOD_RES 2: note = Aib
  MOD_RES 3: note = Glu has side chain protecting group
  MOD_RES 5: note = Thr has side chain protecting group
  MOD_RES 7: note = Thr has side chain protecting group
  MOD_RES 8: note = Ser has side chain protecting group
  MOD_RES 9: note = Asp has side chain protecting group
  MOD_RES 11: note = Ser has side chain protecting group
  MOD_RES 12: note = Ser has side chain protecting group
  MOD_RES 13: note = Tyr has side chain protecting group
  MOD_RES 15: note = Glu has side chain protecting group
  MOD_RES 17: note = Gln has side chain protecting group
  MOD_RES 19: note = Ala-OH or activated carboxylic ester of Ala
  VARIANT 19: note = Ala-OH or activated carboxylic ester of Ala
HXEGTFTSDVSSYLEGQAA

SEQ ID NO: 12 - protein, 19 aa, synthetic construct
  REGION 1..19: note = synthetic peptide
  MOD_RES 1: note = N-alpha-(tert-butoxycarbonyl)-N-tau-trityl-L-histid ine
  MOD_RES 2: note = Aib
  MOD_RES 3: note = (S)-2-amino-5-(tert-butoxy)-5-oxopentanoic acid
  MOD_RES 5: note = O-(tert-butyl)-L-threonine
  MOD_RES 7: note = O-(tert-butyl)-L-threonine
  MOD_RES 8: note = O-(tert-butyl)-L-serine
  MOD_RES 9: note = (S)-2-amino-4-(tert-butoxy)-4-oxobutanoic acid
  MOD_RES 11: note = O-(tert-butyl)-L-serine
  MOD_RES 12: note = O-(tert-butyl)-L-serine
  MOD_RES 13: note = (S)-2-amino-3-(4-(tert-butoxy)phenyl)propanoic acid
  MOD_RES 15: note = (S)-2-amino-5-(tert-butoxy)-5-oxopentanoic acid
  MOD_RES 17: note = N5-trityl-L-glutamine
HXEGTFTSDVSSYLEGQAA

SEQ ID NO: 13 - protein, 12 aa, synthetic construct
  REGION 1..12: note = synthetic peptide
  MOD_RES 1: note = Lys(N-(17-carboxy-1-oxoheptadecyl)-L-gamma-glutamyl -2-[2-(2-amino ethoxy)ethoxy]acetyl-2-[2-(2-aminoethoxy)ethoxy]acetyl)
  MOD_RES 2: note = Glu has side chain protecting group
  MOD_RES 6: note = Trp has side chain protecting group
  MOD_RES 9: note = Arg has side chain protecting group
  MOD_RES 11: note = Arg has side chain protecting group
  MOD_RES 12: note = Gly-O-carboxy protecting group
KEFIAWLVRGRG

SEQ ID NO: 14 - protein, 12 aa, synthetic construct
  REGION 1..12: note = synthetic peptide
  MOD_RES 1: note = Lys(N-(17-carboxy-1-oxoheptadecyl)-L-gamma-glutamyl -2-[2-(2-amino ethoxy)ethoxy]acetyl-2-[2-(2-aminoethoxy)ethoxy]acetyl)
  MOD_RES 2: note = (S)-2-amino-5-(tert-butoxy)-5-oxopentanoic acid
  MOD_RES 6: note = 1-(tert-butoxycarbonyl)-L-tryptophan
  MOD_RES 9: note = N-omega-((2,2,4,6,7-pentamethyl-2,3-dihydrobenzofur an-5-yl)sulfonyl)-L-arginine
  MOD_RES 11: note = N-omega-((2,2,4,6,7-pentamethyl-2,3-dihydrobenzofur an-5-yl)sulfonyl)-L-arginine
KEFIAWLVRGRG

SEQ ID NO: 15 - protein, 18 aa, synthetic construct
  REGION 1..18: note = synthetic peptide
HAEGTFTSDVSSYLEGQA

SEQ ID NO: 16 - protein, 13 aa, synthetic construct
  REGION 1..13: note = synthetic peptide
  MOD_RES 2: note = N6-(4-carboxy-4-palmitamidobutanoyl)lysine or O-protected N6-(4-carboxy-4-palmitamidobutanoyl)lysine
  VARIANT 2: note = N6-(4-carboxy-4-palmitamidobutanoyl)lysine or O-protected N6-(4-carboxy-4-palmitamidobutanoyl)lysine
AKEFIAWLVRGRG

SEQ ID NO: 17 - protein, 18 aa, synthetic construct
  REGION 1..18: note = synthetic peptide
  MOD_RES 1: note = His has N-terminal and side chain protecting groups
  MOD_RES 3: note = Glu has side chain protecting group
  MOD_RES 5: note = Thr has side chain protecting group
  MOD_RES 7: note = Thr has side chain protecting group
  MOD_RES 8: note = Ser has side chain protecting group
  MOD_RES 9: note = Asp has side chain protecting group
  MOD_RES 11: note = Ser has side chain protecting group
  MOD_RES 12: note = Ser has side chain protecting group
  MOD_RES 13: note = Tyr has side chain protecting group
  MOD_RES 15: note = Glu has side chain protecting group
  MOD_RES 17: note = Gln has side chain protecting group
  MOD_RES 18: note = Ala-OH or activated carboxylic ester of Ala
  VARIANT 18: note = Ala-OH or activated carboxylic ester of Ala
HAEGTFTSDVSSYLEGQA

SEQ ID NO: 18 - protein, 18 aa, synthetic construct
  REGION 1..18: note = synthetic peptide
  MOD_RES 1: note = N-alpha-(tert-butoxycarbonyl)-N-tau-trityl-L-histid ine
  MOD_RES 3: note = (S)-2-amino-5-(tert-butoxy)-5-oxopentanoic acid
  MOD_RES 5: note = O-(tert-butyl)-L-threonine
  MOD_RES 7: note = O-(tert-butyl)-L-threonine
  MOD_RES 8: note = O-(tert-butyl)-L-serine
  MOD_RES 9: note = (S)-2-amino-4-(tert-butoxy)-4-oxobutanoic acid
  MOD_RES 11: note = O-(tert-butyl)-L-serine
  MOD_RES 12: note = O-(tert-butyl)-L-serine
  MOD_RES 13: note = (S)-2-amino-3-(4-(tert-butoxy)phenyl)propanoic acid
  MOD_RES 15: note = (S)-2-amino-5-(tert-butoxy)-5-oxopentanoic acid
  MOD_RES 17: note = N5-trityl-L-glutamine
HAEGTFTSDVSSYLEGQA

SEQ ID NO: 19 - protein, 13 aa, synthetic construct
  REGION 1..13: note = synthetic peptide
  MOD_RES 2: note = N6-(4-carboxy-4-palmitamidobutanoyl)lysine
  MOD_RES 3: note = Glu has side chain protecting group
  MOD_RES 7: note = Trp has side chain protecting group
  MOD_RES 12: note = Arg has side chain protecting group
  MOD_RES 13: note = Gly-O-carboxy protecting group
AKEFIAWLVRGRG

SEQ ID NO: 20 - protein, 13 aa, synthetic construct
  REGION 1..13: note = synthetic peptide
  MOD_RES 2: note = N6-(4-carboxy-4-palmitamidobutanoyl)lysine
  MOD_RES 3: note = (S)-2-amino-5-(tert-butoxy)-5-oxopentanoic acid
  MOD_RES 7: note = 1-(tert-butoxycarbonyl)-L-tryptophan
  MOD_RES 10: note = N-omega-((2,2,4,6,7-pentamethyl-2,3-dihydrobenzofur an-5-yl)sulfonyl)-L-arginine
  MOD_RES 12: note = N-omega-((2,2,4,6,7-pentamethyl-2,3-dihydrobenzofur an-5-yl)sulfonyl)-L-arginine
AKEFIAWLVRGRG

SEQ ID NO: 21 - protein, 18 aa, synthetic construct
  REGION 1..18: note = synthetic peptide
  MOD_RES 2: note = Aib
HXEGTFTSDVSSYLEGQA

SEQ ID NO: 22 - protein, 13 aa, synthetic construct
  REGION 1..13: note = synthetic peptide
  MOD_RES 2: note = Lys(N-(17-carboxy-1-oxoheptadecyl)-L-gamma-glutamyl -2-[2-(2-amino ethoxy)ethoxy]acetyl-2-[2-(2-aminoethoxy)ethoxy]acetyl)
AKEFIAWLVRGRG

SEQ ID NO: 23 - protein, 18 aa, synthetic construct
  REGION 1..18: note = synthetic peptide
  MOD_RES 1: note = His has N-terminal and side chain protecting groups
  MOD_RES 2: note = Aib
  MOD_RES 3: note = Glu has side chain protecting group
  MOD_RES 5: note = Thr has side chain protecting group
  MOD_RES 7: note = Thr has side chain protecting group
  MOD_RES 8: note = Ser has side chain protecting group
  MOD_RES 9: note = Asp has side chain protecting group
  MOD_RES 11: note = Ser has side chain protecting group
  MOD_RES 12: note = Ser has side chain protecting group
  MOD_RES 13: note = Tyr has side chain protecting group
  MOD_RES 15: note = Glu has side chain protecting group
  MOD_RES 17: note = Gln has side chain protecting group
  MOD_RES 18: note = Ala-OH or activated carboxylic ester of Ala
  VARIANT 18: note = Ala-OH or activated carboxylic ester of Ala
HXEGTFTSDVSSYLEGQA

SEQ ID NO: 24 - protein, 19 aa, synthetic construct
  REGION 1..19: note = synthetic peptide
  MOD_RES 1: note = N-alpha-(tert-butoxycarbonyl)-N-tau-trityl-L-histid ine
  MOD_RES 2: note = Aib
  MOD_RES 3: note = (S)-2-amino-5-(tert-butoxy)-5-oxopentanoic acid
  MOD_RES 5: note = O-(tert-butyl)-L-threonine
  MOD_RES 7: note = O-(tert-butyl)-L-threonine
  MOD_RES 8: note = O-(tert-butyl)-L-serine
  MOD_RES 9: note = (S)-2-amino-4-(tert-butoxy)-4-oxobutanoic acid
  MOD_RES 11: note = O-(tert-butyl)-L-serine
  MOD_RES 12: note = O-(tert-butyl)-L-serine
  MOD_RES 13: note = (S)-2-amino-3-(4-(tert-butoxy)phenyl)propanoic acid
  MOD_RES 15: note = (S)-2-amino-5-(tert-butoxy)-5-oxopentanoic acid
  MOD_RES 17: note = N5-trityl-L-glutamine
HXEGTFTSDVSSYLEGQAA

SEQ ID NO: 25 - protein, 13 aa, synthetic construct
  REGION 1..13: note = synthetic peptide
  MOD_RES 2: note = Lys(N-(17-carboxy-1-oxoheptadecyl)-L-gamma-glutamyl -2-[2-(2-amino ethoxy)ethoxy]acetyl-2-[2-(2-aminoethoxy)ethoxy]acetyl)
  MOD_RES 3: note = Glu has side chain protecting group
  MOD_RES 7: note = Trp has side chain protecting group
  MOD_RES 10: note = Arg has side chain protecting group
  MOD_RES 12: note = Arg has side chain protecting group
  MOD_RES 13: note = Gly-O-carboxy protecting group
AKEFIAWLVRGRG

SEQ ID NO: 26 - protein, 13 aa, synthetic construct
  REGION 1..13: note = synthetic peptide
  MOD_RES 2: note = Lys(N-(17-carboxy-1-oxoheptadecyl)-L-gamma-glutamyl -2-[2-(2-amino ethoxy)ethoxy]acetyl-2-[2-(2-aminoethoxy)ethoxy]acetyl)
  MOD_RES 3: note = (S)-2-amino-5-(tert-butoxy)-5-oxopentanoic acid
  MOD_RES 7: note = 1-(tert-butoxycarbonyl)-L-tryptophan
  MOD_RES 10: note = N-omega-((2,2,4,6,7-pentamethyl-2,3-dihydrobenzofur an-5-yl)sulfonyl)-L-arginine
  MOD_RES 12: note = N-omega-((2,2,4,6,7-pentamethyl-2,3-dihydrobenzofur an-5-yl)sulfonyl)-L-arginine
AKEFIAWLVRGRG

SEQ ID NO: 27 - protein, 17 aa, synthetic construct
  REGION 1..17: note = synthetic peptide
HAEGTFTSDVSSYLEGQ

SEQ ID NO: 28 - protein, 14 aa, synthetic construct
  REGION 1..14: note = synthetic peptide
  MOD_RES 3: note = N6-(4-carboxy-4-palmitamidobutanoyl)lysine or O-protected N6-(4-carboxy-4-palmitamidobutanoyl)lysine
  VARIANT 3: note = N6-(4-carboxy-4-palmitamidobutanoyl)lysine or O-protected N6-(4-carboxy-4-palmitamidobutanoyl)lysine
AAKEFIAWLVRGRG

SEQ ID NO: 29 - protein, 17 aa, synthetic construct
  REGION 1..17: note = synthetic peptide
  MOD_RES 1: note = His has N-terminal and side chain protecting groups
  MOD_RES 3: note = Glu has side chain protecting group
  MOD_RES 5: note = Thr has side chain protecting group
  MOD_RES 7: note = Thr has side chain protecting group
  MOD_RES 8: note = Ser has side chain protecting group
  MOD_RES 9: note = Asp has side chain protecting group
  MOD_RES 11: note = Ser has side chain protecting group
  MOD_RES 12: note = Ser has side chain protecting group
  MOD_RES 13: note = Tyr has side chain protecting group
  MOD_RES 15: note = Glu has side chain protecting group
  MOD_RES 17: note = Gln-OH or activated carboxylic ester of Gln
  VARIANT 17: note = Gln-OH or activated carboxylic ester of Gln
HAEGTFTSDVSSYLEGQ

SEQ ID NO: 30 - protein, 17 aa, synthetic construct
  REGION 1..17: note = synthetic peptide
  MOD_RES 1: note = N-alpha-(tert-butoxycarbonyl)-N-tau-trityl-L-histid ine
  MOD_RES 3: note = (S)-2-amino-5-(tert-butoxy)-5-oxopentanoic acid
  MOD_RES 5: note = O-(tert-butyl)-L-threonine
  MOD_RES 7: note = O-(tert-butyl)-L-threonine
  MOD_RES 8: note = O-(tert-butyl)-L-serine
  MOD_RES 9: note = (S)-2-amino-4-(tert-butoxy)-4-oxobutanoic acid
  MOD_RES 11: note = O-(tert-butyl)-L-serine
  MOD_RES 12: note = O-(tert-butyl)-L-serine
  MOD_RES 13: note = (S)-2-amino-3-(4-(tert-butoxy)phenyl)propanoic acid
  MOD_RES 15: note = (S)-2-amino-5-(tert-butoxy)-5-oxopentanoic acid
HAEGTFTSDVSSYLEGQ

SEQ ID NO: 31 - protein, 14 aa, synthetic construct
  REGION 1..14: note = synthetic peptide
  MOD_RES 3: note = N6-(4-carboxy-4-palmitamidobutanoyl)lysine
  MOD_RES 4: note = Glu has side chain protecting group
  MOD_RES 8: note = Trp has side chain protecting group
  MOD_RES 11: note = Arg has side chain protecting group
  MOD_RES 13: note = Arg has side chain protecting group
  MOD_RES 14: note = Gly-O-carboxy protecting group
AAKEFIAWLVRGRG

SEQ ID NO: 32 - protein, 14 aa, synthetic construct
  REGION 1..14: note = synthetic peptide
  MOD_RES 3: note = N6-(4-carboxy-4-palmitamidobutanoyl)lysine
  MOD_RES 4: note = (S)-2-amino-5-(tert-butoxy)-5-oxopentanoic acid
  MOD_RES 8: note = 1-(tert-butoxycarbonyl)-L-tryptophan
  MOD_RES 11: note = N-omega-((2,2,4,6,7-pentamethyl-2,3-dihydrobenzofur an-5-yl)sulfonyl)-L-arginine
  MOD_RES 13: note = N-omega-((2,2,4,6,7-pentamethyl-2,3-dihydrobenzofur an-5-yl)sulfonyl)-L-arginine
AAKEFIAWLVRGRG

SEQ ID NO: 33 - protein, 17 aa, synthetic construct
  REGION 1..17: note = synthetic peptide
  MOD_RES 2: note = Aib
HXEGTFTSDVSSYLEGQ

SEQ ID NO: 34 - protein, 14 aa, synthetic construct
  REGION 1..14: note = synthetic peptide
  MOD_RES 3: note = Lys(N-(17-carboxy-1-oxoheptadecyl)-L-gamma-glutamyl -2-[2-(2-amino ethoxy)ethoxy]acetyl-2-[2-(2-aminoethoxy)ethoxy]acetyl)
AAKEFIAWLVRGRG

SEQ ID NO: 35 - protein, 17 aa, synthetic construct
  REGION 1..17: note = synthetic peptide
  MOD_RES 1: note = His has N-terminal and side chain protecting groups
  MOD_RES 2: note = Aib
  MOD_RES 3: note = Glu has side chain protecting group
  MOD_RES 5: note = Thr has side chain protecting group
  MOD_RES 7: note = Thr has side chain protecting group
  MOD_RES 8: note = Ser has side chain protecting group
  MOD_RES 9: note = Asp has side chain protecting group
  MOD_RES 11: note = Ser has side chain protecting group
  MOD_RES 12: note = Ser has side chain protecting group
  MOD_RES 13: note = Tyr has side chain protecting group
  MOD_RES 15: note = Glu has side chain protecting group
  MOD_RES 17: note = Gln-OH or activated carboxylic ester of Gln
  VARIANT 17: note = Gln-OH or activated carboxylic ester of Gln
HXEGTFTSDVSSYLEGQ

SEQ ID NO: 36 - protein, 17 aa, synthetic construct
  REGION 1..17: note = synthetic peptide
  MOD_RES 1: note = N-alpha-(tert-butoxycarbonyl)-N-tau-trityl-L-histid ine
  MOD_RES 2: note = Aib
  MOD_RES 3: note = (S)-2-amino-5-(tert-butoxy)-5-oxopentanoic acid
  MOD_RES 5: note = O-(tert-butyl)-L-threonine
  MOD_RES 7: note = O-(tert-butyl)-L-threonine
  MOD_RES 8: note = O-(tert-butyl)-L-serine
  MOD_RES 9: note = (S)-2-amino-4-(tert-butoxy)-4-oxobutanoic acid
  MOD_RES 11: note = O-(tert-butyl)-L-serine
  MOD_RES 12: note = O-(tert-butyl)-L-serine
  MOD_RES 13: note = (S)-2-amino-3-(4-(tert-butoxy)phenyl)propanoic acid
  MOD_RES 15: note = (S)-2-amino-5-(tert-butoxy)-5-oxopentanoic acid
HXEGTFTSDVSSYLEGQ

SEQ ID NO: 37 - protein, 14 aa, synthetic construct
  REGION 1..14: note = synthetic peptide
  MOD_RES 3: note = Lys(N-(17-carboxy-1-oxoheptadecyl)-L-gamma-glutamyl -2-[2-(2-amino ethoxy)ethoxy]acetyl-2-[2-(2-aminoethoxy)ethoxy]acetyl)
  MOD_RES 4: note = Glu has side chain protecting group
  MOD_RES 8: note = Trp has side chain protecting group
  MOD_RES 11: note = Arg has side chain protecting group
  MOD_RES 13: note = Arg has side chain protecting group
  MOD_RES 14: note = Gly-O-carboxy protecting group
AAKEFIAWLVRGRG

SEQ ID NO: 38 - protein, 14 aa, synthetic construct
  REGION 1..14: note = synthetic peptide
  MOD_RES 3: note = Lys(N-(17-carboxy-1-oxoheptadecyl)-L-gamma-glutamyl -2-[2-(2-amino ethoxy)ethoxy]acetyl-2-[2-(2-aminoethoxy)ethoxy]acetyl)
  MOD_RES 4: note = (S)-2-amino-5-(tert-butoxy)-5-oxopentanoic acid
  MOD_RES 8: note = 1-(tert-butoxycarbonyl)-L-tryptophan
  MOD_RES 11: note = N-omega-((2,2,4,6,7-pentamethyl-2,3-dihydrobenzofur an-5-yl)sulfonyl)-L-arginine
  MOD_RES 13: note = N-omega-((2,2,4,6,7-pentamethyl-2,3-dihydrobenzofur an-5-yl)sulfonyl)-L-arginine
AAKEFIAWLVRGRG

SEQ ID NO: 39 - protein, 14 aa, synthetic construct
  REGION 1..14: note = synthetic peptide
HAEGTFTSDVSSYL

SEQ ID NO: 40 - protein, 17 aa, synthetic construct
  REGION 1..17: note = synthetic peptide
  MOD_RES 6: note = N6-(4-carboxy-4-palmitamidobutanoyl)lysine or O-protected N6-(4-carboxy-4-palmitamidobutanoyl)lysine
  VARIANT 6: note = N6-(4-carboxy-4-palmitamidobutanoyl)lysine or O-protected N6-(4-carboxy-4-palmitamidobutanoyl)lysine
EGQAAKEFIAWLVRGRG

SEQ ID NO: 41 - protein, 14 aa, synthetic construct
  REGION 1..14: note = synthetic peptide
  MOD_RES 1: note = His has N-terminal and side chain protecting groups
  MOD_RES 3: note = Glu has side chain protecting group
  MOD_RES 5: note = Thr has side chain protecting group
  MOD_RES 7: note = Thr has side chain protecting group
  MOD_RES 8: note = Ser has side chain protecting group
  MOD_RES 9: note = Asp has side chain protecting group
  MOD_RES 11: note = Ser has side chain protecting group
  MOD_RES 12: note = Ser has side chain protecting group
  MOD_RES 13: note = Tyr has side chain protecting group
  MOD_RES 14: note = Leu-OH or activated carboxylic ester of Leu
  VARIANT 14: note = Leu-OH or activated carboxylic ester of Leu
HAEGTFTSDVSSYL

SEQ ID NO: 42 - protein, 14 aa, synthetic construct
  REGION 1..14: note = synthetic peptide
  MOD_RES 1: note = N-alpha-(tert-butoxycarbonyl)-N-tau-trityl-L-histid ine
  MOD_RES 3: note = (S)-2-amino-5-(tert-butoxy)-5-oxopentanoic acid
  MOD_RES 5: note = O-(tert-butyl)-L-threonine
  MOD_RES 7: note = O-(tert-butyl)-L-threonine
  MOD_RES 8: note = O-(tert-butyl)-L-serine
  MOD_RES 9: note = (S)-2-amino-4-(tert-butoxy)-4-oxobutanoic acid
  MOD_RES 11: note = O-(tert-butyl)-L-serine
  MOD_RES 12: note = O-(tert-butyl)-L-serine
  MOD_RES 13: note = (S)-2-amino-3-(4-(tert-butoxy)phenyl)propanoic acid
HAEGTFTSDVSSYL

SEQ ID NO: 43 - protein, 17 aa, synthetic construct
  REGION 1..17: note = synthetic peptide
  MOD_RES 1: note = Glu has side chain protecting group
  MOD_RES 3: note = Gln has side chain protecting group
  MOD_RES 6: note = N6-(4-carboxy-4-palmitamidobutanoyl)lysine
  MOD_RES 7: note = Glu has side chain protecting group
  MOD_RES 11: note = Trp has side chain protecting group
  MOD_RES 14: note = Arg has side chain protecting group
  MOD_RES 16: note = Arg has side chain protecting group
  MOD_RES 17: note = Gly-O-carboxy protecting group
EGQAAKEFIAWLVRGRG

SEQ ID NO: 44 - protein, 18 aa, synthetic construct
  REGION 1..18: note = synthetic peptide
  MOD_RES 1: note = (S)-2-amino-5-(tert-butoxy)-5-oxopentanoic acid
  MOD_RES 3: note = N5-trityl-L-glutamine
  MOD_RES 7: note = N6-(4-carboxy-4-palmitamidobutanoyl)lysine
  MOD_RES 8: note = (S)-2-amino-5-(tert-butoxy)-5-oxopentanoic acid
  MOD_RES 12: note = 1-(tert-butoxycarbonyl)-L-tryptophan
  MOD_RES 15: note = N-omega-((2,2,4,6,7-pentamethyl-2,3-dihydrobenzofur an-5-yl)sulfonyl)-L-arginine
  MOD_RES 17: note = N-omega-((2,2,4,6,7-pentamethyl-2,3-dihydrobenzofur an-5-yl)sulfonyl)-L-arginine
EGQAAAKEFIAWLVRGRG

SEQ ID NO: 45 - protein, 14 aa, synthetic construct
  REGION 1..14: note = synthetic peptide
  MOD_RES 2: note = Aib
HXEGTFTSDVSSYL

SEQ ID NO: 46 - protein, 17 aa, synthetic construct
  REGION 1..17: note = synthetic peptide
  MOD_RES 6: note = Lys(N-(17-carboxy-1-oxoheptadecyl)-L-gamma-glutamyl -2-[2-(2-amino ethoxy)ethoxy]acetyl-2-[2-(2-aminoethoxy)ethoxy]acetyl)
EGQAAKEFIAWLVRGRG

SEQ ID NO: 47 - protein, 14 aa, synthetic construct
  REGION 1..14: note = synthetic peptide
  MOD_RES 1: note = His has N-terminal and side chain protecting groups
  MOD_RES 2: note = Aib
  MOD_RES 3: note = Glu has side chain protecting group
  MOD_RES 5: note = Thr has side chain protecting group
  MOD_RES 7: note = Thr has side chain protecting group
  MOD_RES 8: note = Ser has side chain protecting group
  MOD_RES 9: note = Asp has side chain protecting group
  MOD_RES 11: note = Ser has side chain protecting group
  MOD_RES 12: note = Ser has side chain protecting group
  MOD_RES 13: note = Tyr has side chain protecting group
  MOD_RES 14: note = Leu-OH or activated carboxylic ester of Leu
  VARIANT 14: note = Leu-OH or activated carboxylic ester of Leu
HXEGTFTSDVSSYL

SEQ ID NO: 48 - protein, 14 aa, synthetic construct
  REGION 1..14: note = synthetic peptide
  MOD_RES 1: note = N-alpha-(tert-butoxycarbonyl)-N-tau-trityl-L-histid ine
  MOD_RES 2: note = Aib
  MOD_RES 3: note = (S)-2-amino-5-(tert-butoxy)-5-oxopentanoic acid
  MOD_RES 5: note = O-(tert-butyl)-L-threonine
  MOD_RES 7: note = O-(tert-butyl)-L-threonine
  MOD_RES 8: note = O-(tert-butyl)-L-serine
  MOD_RES 9: note = (S)-2-amino-4-(tert-butoxy)-4-oxobutanoic acid
  MOD_RES 11: note = O-(tert-butyl)-L-serine
  MOD_RES 12: note = O-(tert-butyl)-L-serine
  MOD_RES 13: note = (S)-2-amino-3-(4-(tert-butoxy)phenyl)propanoic acid
HXEGTFTSDVSSYL

SEQ ID NO: 49 - protein, 17 aa, synthetic construct
  REGION 1..17: note = synthetic peptide
  MOD_RES 1: note = Glu has side chain protecting group
  MOD_RES 3: note = Gln has side chain protecting group
  MOD_RES 6: note = Lys(N-(17-carboxy-1-oxoheptadecyl)-L-gamma-glutamyl -2-[2-(2-amino ethoxy)ethoxy]acetyl-2-[2-(2-aminoethoxy)ethoxy]acetyl)
  MOD_RES 7: note = Glu has side chain protecting group
  MOD_RES 11: note = Trp has side chain protecting group
  MOD_RES 14: note = Arg has side chain protecting group
  MOD_RES 16: note = Arg has side chain protecting group
  MOD_RES 17: note = Gly-O-carboxy protecting group
EGQAAKEFIAWLVRGRG

SEQ ID NO: 50 - protein, 16 aa, synthetic construct
  REGION 1..16: note = synthetic peptide
  MOD_RES 1: note = (S)-2-amino-5-(tert-butoxy)-5-oxopentanoic acid
  MOD_RES 3: note = N5-trityl-L-glutamine
  MOD_RES 5: note = Lys(N-(17-carboxy-1-oxoheptadecyl)-L-gamma-glutamyl -2-[2-(2-amino ethoxy)ethoxy]acetyl-2-[2-(2-aminoethoxy)ethoxy]acetyl)
  MOD_RES 6: note = (S)-2-amino-5-(tert-butoxy)-5-oxopentanoic acid
  MOD_RES 10: note = 1-(tert-butoxycarbonyl)-L-tryptophan
  MOD_RES 13: note = N-omega-((2,2,4,6,7-pentamethyl-2,3-dihydrobenzofur an-5-yl)sulfonyl)-L-arginine
  MOD_RES 15: note = N-omega-((2,2,4,6,7-pentamethyl-2,3-dihydrobenzofur an-5-yl)sulfonyl)-L-arginine
EGQAKEFIAWLVRGRG

SEQ ID NO: 51 - protein, 4 aa, synthetic construct
  REGION 1..4: note = synthetic peptide
HAEG

SEQ ID NO: 52 - protein, 4 aa, synthetic construct
  REGION 1..4: note = synthetic peptide
  MOD_RES 1: note = His has N-terminal protecting group
HAEG

SEQ ID NO: 53 - protein, 15 aa, synthetic construct
  REGION 1..15: note = synthetic peptide
  MOD_RES 15: note = Ala has C-terminal protecting group
TFTSDVSSYLEGQAA

SEQ ID NO: 54-55 - skipped (2 entries)

SEQ ID NO: 56 - protein, 15 aa, synthetic construct
  REGION 1..15: note = synthetic peptide
  MOD_RES 15: note = Ala has C-terminal protecting group
TFTSDVSSYLEGQAA

SEQ ID NO: 57-58 - skipped (2 entries)

SEQ ID NO: 59 - protein, 19 aa, synthetic construct
  REGION 1..19: note = synthetic peptide
  MOD_RES 1: note = N-alpha-(tert-butoxycarbonyl)-N-tau-trityl-L-histid ine
  MOD_RES 3: note = (S)-2-amino-5-(tert-butoxy)-5-oxopentanoic acid
  MOD_RES 5: note = O-(tert-butyl)-L-threonine
  MOD_RES 7: note = O-(tert-butyl)-L-threonine
  MOD_RES 8: note = O-(tert-butyl)-L-serine
  MOD_RES 9: note = (S)-2-amino-4-(tert-butoxy)-4-oxobutanoic acid
  MOD_RES 11: note = O-(tert-butyl)-L-serine
  MOD_RES 12: note = Serine pseudoproline
  MOD_RES 13: note = (S)-2-amino-3-(4-(tert-butoxy)phenyl)propanoic acid
  MOD_RES 15: note = (S)-2-amino-5-(tert-butoxy)-5-oxopentanoic acid
  MOD_RES 17: note = N5-trityl-L-glutamine
HAEGTFTSDVSSYLEGQAA

SEQ ID NO: 60 - protein, 19 aa, synthetic construct
  REGION 1..19: note = synthetic peptide
  MOD_RES 1: note = N-alpha-(tert-butoxycarbonyl)-N-tau-trityl-L-histid ine
  MOD_RES 2: note = Aib
  MOD_RES 3: note = (S)-2-amino-5-(tert-butoxy)-5-oxopentanoic acid
  MOD_RES 5: note = O-(tert-butyl)-L-threonine
  MOD_RES 7: note = O-(tert-butyl)-L-threonine
  MOD_RES 8: note = O-(tert-butyl)-L-serine
  MOD_RES 9: note = (S)-2-amino-4-(tert-butoxy)-4-oxobutanoic acid
  MOD_RES 11: note = O-(tert-butyl)-L-serine
  MOD_RES 12: note = Serine pseudoproline
  MOD_RES 13: note = (S)-2-amino-3-(4-(tert-butoxy)phenyl)propanoic acid
  MOD_RES 15: note = (S)-2-amino-5-(tert-butoxy)-5-oxopentanoic acid
  MOD_RES 17: note = N5-trityl-L-glutamine
HXEGTFTSDVSSYLEGQAA

SEQ ID NO: 61 - protein, 19 aa, synthetic construct
  REGION 1..19: note = synthetic peptide
  MOD_RES 1: note = N-alpha-(tert-butoxycarbonyl)-N-tau-trityl-L-histid ine
  MOD_RES 2: note = Aib
  MOD_RES 3: note = (S)-2-amino-5-(tert-butoxy)-5-oxopentanoic acid
  MOD_RES 5: note = O-(tert-butyl)-L-threonine
  MOD_RES 7: note = Threonine pseudoproline
  MOD_RES 8: note = O-(tert-butyl)-L-serine
  MOD_RES 9: note = (S)-2-amino-4-(tert-butoxy)-4-oxobutanoic acid
  MOD_RES 11: note = O-(tert-butyl)-L-serine
  MOD_RES 12: note = Serine pseudoproline
  MOD_RES 13: note = (S)-2-amino-3-(4-(tert-butoxy)phenyl)propanoic acid
  MOD_RES 15: note = (S)-2-amino-5-(tert-butoxy)-5-oxopentanoic acid
  MOD_RES 17: note = N5-trityl-L-glutamine
HXEGTFTSDVSSYLEGQAA

SEQ ID NO: 62 - protein, 19 aa, synthetic construct
  REGION 1..19: note = synthetic peptide
  MOD_RES 1: note = N-alpha-(tert-butoxycarbonyl)-N-tau-trityl-L-histid ine
  MOD_RES 3: note = (S)-2-amino-5-(tert-butoxy)-5-oxopentanoic acid
  MOD_RES 5: note = O-(tert-butyl)-L-threonine
  MOD_RES 7: note = Threonine pseudoproline
  MOD_RES 8: note = O-(tert-butyl)-L-serine
  MOD_RES 9: note = (S)-2-amino-4-(tert-butoxy)-4-oxobutanoic acid
  MOD_RES 11: note = O-(tert-butyl)-L-serine
  MOD_RES 12: note = Serine pseudoproline
  MOD_RES 13: note = (S)-2-amino-3-(4-(tert-butoxy)phenyl)propanoic acid
  MOD_RES 15: note = (S)-2-amino-5-(tert-butoxy)-5-oxopentanoic acid
  MOD_RES 17: note = N5-trityl-L-glutamine
HAEGTFTSDVSSYLEGQAA

SEQ ID NO: 63 - protein, 12 aa, synthetic construct
  REGION 1..12: note = synthetic peptide
  MOD_RES 1: note = N6-(4-carboxy-4-palmitamidobutanoyl)lysine
  MOD_RES 2: note = (S)-2-amino-5-(tert-butoxy)-5-oxopentanoic acid
  MOD_RES 6: note = 1-(tert-butoxycarbonyl)-L-tryptophan
  MOD_RES 9: note = N-omega-((2,2,4,6,7-pentamethyl-2,3-dihydrobenzofur an-5-yl)sulfonyl)-L-arginine
  MOD_RES 11: note = N-omega-((2,2,4,6,7-pentamethyl-2,3-dihydrobenzofur an-5-yl)sulfonyl)-L-arginine
KEFIAWLVRGRG

SEQ ID NO: 64 - protein, 12 aa, synthetic construct
  REGION 1..12: note = synthetic peptide
  MOD_RES 1: note = N6-(4-carboxy-4-palmitamidobutanoyl)lysine
  MOD_RES 2: note = (S)-2-amino-5-(tert-butoxy)-5-oxopentanoic acid
  MOD_RES 6: note = 1-(tert-butoxycarbonyl)-L-tryptophan
  MOD_RES 9: note = N-omega-((2,2,4,6,7-pentamethyl-2,3-dihydrobenzofur an-5-yl)sulfonyl)-L-arginine
  MOD_RES 11: note = N-omega-((2,2,4,6,7-pentamethyl-2,3-dihydrobenzofur an-5-yl)sulfonyl)-L-arginine
KEFIAWLVRGRG

SEQ ID NO: 65 - protein, 18 aa, synthetic construct
  REGION 1..18: note = synthetic peptide
  MOD_RES 1: note = N-alpha-(tert-butoxycarbonyl)-N-tau-trityl-L-histid ine
  MOD_RES 3: note = (S)-2-amino-5-(tert-butoxy)-5-oxopentanoic acid
  MOD_RES 5: note = O-(tert-butyl)-L-threonine
  MOD_RES 7: note = O-(tert-butyl)-L-threonine
  MOD_RES 8: note = O-(tert-butyl)-L-serine
  MOD_RES 9: note = (S)-2-amino-4-(tert-butoxy)-4-oxobutanoic acid
  MOD_RES 11: note = O-(tert-butyl)-L-serine
  MOD_RES 12: note = Serine pseudoproline
  MOD_RES 13: note = (S)-2-amino-3-(4-(tert-butoxy)phenyl)propanoic acid
  MOD_RES 15: note = (S)-2-amino-5-(tert-butoxy)-5-oxopentanoic acid
  MOD_RES 17: note = N5-trityl-L-glutamine
HAEGTFTSDVSSYLEGQA

SEQ ID NO: 66 - protein, 18 aa, synthetic construct
  REGION 1..18: note = synthetic peptide
  MOD_RES 1: note = N-alpha-(tert-butoxycarbonyl)-N-tau-trityl-L-histid ine
  MOD_RES 3: note = (S)-2-amino-5-(tert-butoxy)-5-oxopentanoic acid
  MOD_RES 5: note = O-(tert-butyl)-L-threonine
  MOD_RES 7: note = Threonine pseudoproline
  MOD_RES 8: note = O-(tert-butyl)-L-serine
  MOD_RES 9: note = (S)-2-amino-4-(tert-butoxy)-4-oxobutanoic acid
  MOD_RES 11: note = O-(tert-butyl)-L-serine
  MOD_RES 12: note = Serine pseudoproline
  MOD_RES 13: note = (S)-2-amino-3-(4-(tert-butoxy)phenyl)propanoic acid
  MOD_RES 15: note = (S)-2-amino-5-(tert-butoxy)-5-oxopentanoic acid
  MOD_RES 17: note = N5-trityl-L-glutamine
HAEGTFTSDVSSYLEGQA

SEQ ID NO: 67 - protein, 13 aa, synthetic construct
  REGION 1..13: note = synthetic peptide
  MOD_RES 2: note = N6-(4-carboxy-4-palmitamidobutanoyl)lysine
  MOD_RES 3: note = (S)-2-amino-5-(tert-butoxy)-5-oxopentanoic acid
  MOD_RES 7: note = 1-(tert-butoxycarbonyl)-L-tryptophan
  MOD_RES 10: note = N-omega-((2,2,4,6,7-pentamethyl-2,3-dihydrobenzofur an-5-yl)sulfonyl)-L-arginine
  MOD_RES 12: note = N-omega-((2,2,4,6,7-pentamethyl-2,3-dihydrobenzofur an-5-yl)sulfonyl)-L-arginine
AKEFIAWLVRGRG

SEQ ID NO: 68 - protein, 19 aa, synthetic construct
  REGION 1..19: note = synthetic peptide
  MOD_RES 1: note = N-alpha-(tert-butoxycarbonyl)-N-tau-trityl-L-histid ine
  MOD_RES 2: note = Aib
  MOD_RES 3: note = (S)-2-amino-5-(tert-butoxy)-5-oxopentanoic acid
  MOD_RES 5: note = O-(tert-butyl)-L-threonine
  MOD_RES 7: note = O-(tert-butyl)-L-threonine
  MOD_RES 8: note = O-(tert-butyl)-L-serine
  MOD_RES 9: note = (S)-2-amino-4-(tert-butoxy)-4-oxobutanoic acid
  MOD_RES 11: note = O-(tert-butyl)-L-serine
  MOD_RES 12: note = Serine pseudoproline
  MOD_RES 13: note = (S)-2-amino-3-(4-(tert-butoxy)phenyl)propanoic acid
  MOD_RES 15: note = (S)-2-amino-5-(tert-butoxy)-5-oxopentanoic acid
  MOD_RES 17: note = N5-trityl-L-glutamine
HXEGTFTSDVSSYLEGQAA

SEQ ID NO: 69 - protein, 19 aa, synthetic construct
  REGION 1..19: note = synthetic peptide
  MOD_RES 1: note = N-alpha-(tert-butoxycarbonyl)-N-tau-trityl-L-histid ine
  MOD_RES 2: note = Aib
  MOD_RES 3: note = (S)-2-amino-5-(tert-butoxy)-5-oxopentanoic acid
  MOD_RES 5: note = O-(tert-butyl)-L-threonine
  MOD_RES 7: note = Threonine pseudoproline
  MOD_RES 8: note = O-(tert-butyl)-L-serine
  MOD_RES 9: note = (S)-2-amino-4-(tert-butoxy)-4-oxobutanoic acid
  MOD_RES 11: note = O-(tert-butyl)-L-serine
  MOD_RES 12: note = Serine pseudoproline
  MOD_RES 13: note = (S)-2-amino-3-(4-(tert-butoxy)phenyl)propanoic acid
  MOD_RES 15: note = (S)-2-amino-5-(tert-butoxy)-5-oxopentanoic acid
  MOD_RES 17: note = N5-trityl-L-glutamine
HXEGTFTSDVSSYLEGQAA

SEQ ID NO: 70 - protein, 13 aa, synthetic construct
  REGION 1..13: note = synthetic peptide
  MOD_RES 2: note = Lys(N-(17-carboxy-1-oxoheptadecyl)-L-gamma-glutamyl -2-[2-(2-amino ethoxy)ethoxy]acetyl-2-[2-(2-aminoethoxy)ethoxy]acetyl)
  MOD_RES 3: note = (S)-2-amino-5-(tert-butoxy)-5-oxopentanoic acid
  MOD_RES 7: note = 1-(tert-butoxycarbonyl)-L-tryptophan
  MOD_RES 10: note = N-omega-((2,2,4,6,7-pentamethyl-2,3-dihydrobenzofur an-5-yl)sulfonyl)-L-arginine
  MOD_RES 12: note = N-omega-((2,2,4,6,7-pentamethyl-2,3-dihydrobenzofur an-5-yl)sulfonyl)-L-arginine
AKEFIAWLVRGRG

SEQ ID NO: 71 - protein, 17 aa, synthetic construct
  REGION 1..17: note = synthetic peptide
  MOD_RES 1: note = N-alpha-(tert-butoxycarbonyl)-N-tau-trityl-L-histid ine
  MOD_RES 3: note = (S)-2-amino-5-(tert-butoxy)-5-oxopentanoic acid
  MOD_RES 5: note = O-(tert-butyl)-L-threonine
  MOD_RES 7: note = O-(tert-butyl)-L-threonine
  MOD_RES 8: note = O-(tert-butyl)-L-serine
  MOD_RES 9: note = (S)-2-amino-4-(tert-butoxy)-4-oxobutanoic acid
  MOD_RES 11: note = O-(tert-butyl)-L-serine
  MOD_RES 12: note = Serine pseudoproline
  MOD_RES 13: note = (S)-2-amino-3-(4-(tert-butoxy)phenyl)propanoic acid
  MOD_RES 15: note = (S)-2-amino-5-(tert-butoxy)-5-oxopentanoic acid
HAEGTFTSDVSSYLEGQ

SEQ ID NO: 72 - protein, 17 aa, synthetic construct
  REGION 1..17: note = synthetic peptide
  MOD_RES 1: note = N-alpha-(tert-butoxycarbonyl)-N-tau-trityl-L-histid ine
  MOD_RES 3: note = (S)-2-amino-5-(tert-butoxy)-5-oxopentanoic acid
  MOD_RES 5: note = O-(tert-butyl)-L-threonine
  MOD_RES 7: note = Threonine pseudoproline
  MOD_RES 8: note = O-(tert-butyl)-L-serine
  MOD_RES 9: note = (S)-2-amino-4-(tert-butoxy)-4-oxobutanoic acid
  MOD_RES 11: note = O-(tert-butyl)-L-serine
  MOD_RES 12: note = Serine pseudoproline
  MOD_RES 13: note = (S)-2-amino-3-(4-(tert-butoxy)phenyl)propanoic acid
  MOD_RES 15: note = (S)-2-amino-5-(tert-butoxy)-5-oxopentanoic acid
HAEGTFTSDVSSYLEGQ

SEQ ID NO: 73 - protein, 14 aa, synthetic construct
  REGION 1..14: note = synthetic peptide
  MOD_RES 3: note = N6-(4-carboxy-4-palmitamidobutanoyl)lysine
  MOD_RES 4: note = (S)-2-amino-5-(tert-butoxy)-5-oxopentanoic acid
  MOD_RES 8: note = 1-(tert-butoxycarbonyl)-L-tryptophan
  MOD_RES 11: note = N-omega-((2,2,4,6,7-pentamethyl-2,3-dihydrobenzofur an-5-yl)sulfonyl)-L-arginine
  MOD_RES 13: note = N-omega-((2,2,4,6,7-pentamethyl-2,3-dihydrobenzofur an-5-yl)sulfonyl)-L-arginine
AAKEFIAWLVRGRG

SEQ ID NO: 74 - protein, 17 aa, synthetic construct
  REGION 1..17: note = synthetic peptide
  MOD_RES 1: note = N-alpha-(tert-butoxycarbonyl)-N-tau-trityl-L-histid ine
  MOD_RES 2: note = Aib
  MOD_RES 3: note = (S)-2-amino-5-(tert-butoxy)-5-oxopentanoic acid
  MOD_RES 5: note = O-(tert-butyl)-L-threonine
  MOD_RES 7: note = O-(tert-butyl)-L-threonine
  MOD_RES 8: note = O-(tert-butyl)-L-serine
  MOD_RES 9: note = (S)-2-amino-4-(tert-butoxy)-4-oxobutanoic acid
  MOD_RES 11: note = O-(tert-butyl)-L-serine
  MOD_RES 12: note = Serine pseudoproline
  MOD_RES 13: note = (S)-2-amino-3-(4-(tert-butoxy)phenyl)propanoic acid
  MOD_RES 15: note = (S)-2-amino-5-(tert-butoxy)-5-oxopentanoic acid
HXEGTFTSDVSSYLEGQ

SEQ ID NO: 75 - protein, 17 aa, synthetic construct
  REGION 1..17: note = synthetic peptide
  MOD_RES 1: note = N-alpha-(tert-butoxycarbonyl)-N-tau-trityl-L-histid ine
  MOD_RES 2: note = Aib
  MOD_RES 3: note = (S)-2-amino-5-(tert-butoxy)-5-oxopentanoic acid
  MOD_RES 5: note = O-(tert-butyl)-L-threonine
  MOD_RES 7: note = Threonine pseudoproline
  MOD_RES 8: note = O-(tert-butyl)-L-serine
  MOD_RES 9: note = (S)-2-amino-4-(tert-butoxy)-4-oxobutanoic acid
  MOD_RES 11: note = O-(tert-butyl)-L-serine
  MOD_RES 12: note = Serine pseudoproline
  MOD_RES 13: note = (S)-2-amino-3-(4-(tert-butoxy)phenyl)propanoic acid
  MOD_RES 15: note = (S)-2-amino-5-(tert-butoxy)-5-oxopentanoic acid
HXEGTFTSDVSSYLEGQ

SEQ ID NO: 76 - protein, 14 aa, synthetic construct
  REGION 1..14: note = synthetic peptide
  MOD_RES 3: note = Lys(N-(17-carboxy-1-oxoheptadecyl)-L-gamma-glutamyl -2-[2-(2-amino ethoxy)ethoxy]acetyl-2-[2-(2-aminoethoxy)ethoxy]acetyl)
  MOD_RES 4: note = (S)-2-amino-5-(tert-butoxy)-5-oxopentanoic acid
  MOD_RES 8: note = 1-(tert-butoxycarbonyl)-L-tryptophan
  MOD_RES 11: note = N-omega-((2,2,4,6,7-pentamethyl-2,3-dihydrobenzofur an-5-yl)sulfonyl)-L-arginine
  MOD_RES 13: note = N-omega-((2,2,4,6,7-pentamethyl-2,3-dihydrobenzofur an-5-yl)sulfonyl)-L-arginine
AAKEFIAWLVRGRG

SEQ ID NO: 77 - protein, 14 aa, synthetic construct
  REGION 1..14: note = synthetic peptide
  MOD_RES 1: note = N-alpha-(tert-butoxycarbonyl)-N-tau-trityl-L-histid ine
  MOD_RES 3: note = (S)-2-amino-5-(tert-butoxy)-5-oxopentanoic acid
  MOD_RES 5: note = O-(tert-butyl)-L-threonine
  MOD_RES 7: note = O-(tert-butyl)-L-threonine
  MOD_RES 8: note = O-(tert-butyl)-L-serine
  MOD_RES 9: note = (S)-2-amino-4-(tert-butoxy)-4-oxobutanoic acid
  MOD_RES 11: note = O-(tert-butyl)-L-serine
  MOD_RES 12: note = Serine pseudoproline
  MOD_RES 13: note = (S)-2-amino-3-(4-(tert-butoxy)phenyl)propanoic acid
HAEGTFTSDVSSYL

SEQ ID NO: 78 - protein, 14 aa, synthetic construct
  REGION 1..14: note = synthetic peptide
  MOD_RES 1: note = N-alpha-(tert-butoxycarbonyl)-N-tau-trityl-L-histid ine
  MOD_RES 3: note = (S)-2-amino-5-(tert-butoxy)-5-oxopentanoic acid
  MOD_RES 5: note = O-(tert-butyl)-L-threonine
  MOD_RES 7: note = Threonine pseudoproline
  MOD_RES 8: note = O-(tert-butyl)-L-serine
  MOD_RES 9: note = (S)-2-amino-4-(tert-butoxy)-4-oxobutanoic acid
  MOD_RES 11: note = O-(tert-butyl)-L-serine
  MOD_RES 12: note = Serine pseudoproline
  MOD_RES 13: note = (S)-2-amino-3-(4-(tert-butoxy)phenyl)propanoic acid
HAEGTFTSDVSSYL

SEQ ID NO: 79 - protein, 18 aa, synthetic construct
  REGION 1..18: note = synthetic peptide
  MOD_RES 1: note = (S)-2-amino-5-(tert-butoxy)-5-oxopentanoic acid
  MOD_RES 3: note = N5-trityl-L-glutamine
  MOD_RES 7: note = N6-(4-carboxy-4-palmitamidobutanoyl)lysine
  MOD_RES 8: note = (S)-2-amino-5-(tert-butoxy)-5-oxopentanoic acid
  MOD_RES 12: note = 1-(tert-butoxycarbonyl)-L-tryptophan
  MOD_RES 15: note = N-omega-((2,2,4,6,7-pentamethyl-2,3-dihydrobenzofur an-5-yl)sulfonyl)-L-arginine
  MOD_RES 17: note = N-omega-((2,2,4,6,7-pentamethyl-2,3-dihydrobenzofur an-5-yl)sulfonyl)-L-arginine
EGQAAAKEFIAWLVRGRG

SEQ ID NO: 80 - protein, 14 aa, synthetic construct
  REGION 1..14: note = synthetic peptide
  MOD_RES 1: note = N-alpha-(tert-butoxycarbonyl)-N-tau-trityl-L-histid ine
  MOD_RES 2: note = Aib
  MOD_RES 3: note = (S)-2-amino-5-(tert-butoxy)-5-oxopentanoic acid
  MOD_RES 5: note = O-(tert-butyl)-L-threonine
  MOD_RES 7: note = O-(tert-butyl)-L-threonine
  MOD_RES 8: note = O-(tert-butyl)-L-serine
  MOD_RES 9: note = (S)-2-amino-4-(tert-butoxy)-4-oxobutanoic acid
  MOD_RES 11: note = O-(tert-butyl)-L-serine
  MOD_RES 12: note = Serine pseudoproline
  MOD_RES 13: note = (S)-2-amino-3-(4-(tert-butoxy)phenyl)propanoic acid
HXEGTFTSDVSSYL

SEQ ID NO: 81 - protein, 14 aa, synthetic construct
  REGION 1..14: note = synthetic peptide
  MOD_RES 1: note = N-alpha-(tert-butoxycarbonyl)-N-tau-trityl-L-histid ine
  MOD_RES 2: note = Aib
  MOD_RES 3: note = (S)-2-amino-5-(tert-butoxy)-5-oxopentanoic acid
  MOD_RES 5: note = O-(tert-butyl)-L-threonine
  MOD_RES 7: note = Threonine pseudoproline
  MOD_RES 8: note = O-(tert-butyl)-L-serine
  MOD_RES 9: note = (S)-2-amino-4-(tert-butoxy)-4-oxobutanoic acid
  MOD_RES 11: note = O-(tert-butyl)-L-serine
  MOD_RES 12: note = Serine pseudoproline
  MOD_RES 13: note = (S)-2-amino-3-(4-(tert-butoxy)phenyl)propanoic acid
HXEGTFTSDVSSYL

SEQ ID NO: 82 - protein, 16 aa, synthetic construct
  REGION 1..16: note = synthetic peptide
  MOD_RES 1: note = (S)-2-amino-5-(tert-butoxy)-5-oxopentanoic acid
  MOD_RES 3: note = N5-trityl-L-glutamine
  MOD_RES 5: note = Lys(N-(17-carboxy-1-oxoheptadecyl)-L-gamma-glutamyl -2-[2-(2-amino ethoxy)ethoxy]acetyl-2-[2-(2-aminoethoxy)ethoxy]acetyl)
  MOD_RES 6: note = (S)-2-amino-5-(tert-butoxy)-5-oxopentanoic acid
  MOD_RES 10: note = 1-(tert-butoxycarbonyl)-L-tryptophan
  MOD_RES 13: note = N-omega-((2,2,4,6,7-pentamethyl-2,3-dihydrobenzofur an-5-yl)sulfonyl)-L-arginine
  MOD_RES 15: note = N-omega-((2,2,4,6,7-pentamethyl-2,3-dihydrobenzofur an-5-yl)sulfonyl)-L-arginine
EGQAKEFIAWLVRGRG

SEQ ID NO: 83 - protein, 33 aa, synthetic construct
  REGION 1..33: note = GLP-2 peptide
HGDGSFSDEMNTILDNLAARDFINWLIQTKITD

SEQ ID NO: 84 - protein, 14 aa, synthetic construct
  REGION 1..14: note = synthetic peptide
HGDGSFSDEMNTIL

SEQ ID NO: 85 - protein, 19 aa, synthetic construct
  REGION 1..19: note = synthetic peptide
DNLAARDFINWLIQTKITD

SEQ ID NO: 86 - protein, 14 aa, synthetic construct
  REGION 1..14: note = synthetic peptide
  MOD_RES 1: note = His has N-terminal and side chain protecting groups
  MOD_RES 3: note = Asp has side chain protecting group
  MOD_RES 5: note = Ser has side chain protecting group
  MOD_RES 7: note = Ser has side chain protecting group
  MOD_RES 8: note = Asp has side chain protecting group
  MOD_RES 9: note = Glu has side chain protecting group
  MOD_RES 11: note = Asn has side chain protecting group
  MOD_RES 12: note = Thr has side chain protecting group
  MOD_RES 14: note = Leu-OH or activated carboxylic ester of Leu
  VARIANT 14: note = Leu-OH or activated carboxylic ester of Leu
HGDGSFSDEMNTIL

SEQ ID NO: 87 - protein, 14 aa, synthetic construct
  REGION 1..14: note = synthetic peptide
  MOD_RES 1: note = N-alpha-(tert-butoxycarbonyl)-N-tau-trityl-L-histid ine
  MOD_RES 3: note = (S)-2-amino-4-(tert-butoxy)-4-oxobutanoic acid
  MOD_RES 5: note = O-(tert-butyl)-L-serine
  MOD_RES 7: note = O-(tert-butyl)-L-serine or Serine pseudoproline
  MOD_RES 8: note = (S)-2-amino-4-(tert-butoxy)-4-oxobutanoic acid
  MOD_RES 9: note = (S)-2-amino-5-(tert-butoxy)-5-oxopentanoic acid
  MOD_RES 11: note = N4-trityl-L-asparagine
  MOD_RES 12: note = O-(tert-butyl)-L-threonine
  VARIANT 7: note = O-(tert-butyl)-L-serine or Serine pseudoproline
HGDGSFSDEMNTIL

SEQ ID NO: 88 - protein, 19 aa, synthetic construct
  REGION 1..19: note = synthetic peptide
  MOD_RES 1: note = Asp has side chain protecting group
  MOD_RES 2: note = Asn has side chain protecting group
  MOD_RES 6: note = Arg has side chain protecting group
  MOD_RES 7: note = Asp has side chain protecting group
  MOD_RES 10: note = Asn has side chain protecting group
  MOD_RES 11: note = Trp has side chain protecting group
  MOD_RES 14: note = Gln has side chain protecting group
  MOD_RES 15: note = Thr has side chain protecting group
  MOD_RES 16: note = Lys has side chain protecting group
  MOD_RES 18: note = Thr has side chain protecting group
  MOD_RES 19: note = Asp has side chain protecting group, and Asp-OH or a carboxy protecting group
  VARIANT 19: note = Asp has side chain protecting group, and Asp-OH or a carboxy protecting group
DNLAARDFINWLIQTKITD

SEQ ID NO: 89 - protein, 19 aa, synthetic construct
  REGION 1..19: note = synthetic peptide
  MOD_RES 1: note = (S)-2-amino-4-(tert-butoxy)-4-oxobutanoic acid
  MOD_RES 2: note = N4-trityl-L-asparagine
  MOD_RES 6: note = N-omega-((2,2,4,6,7-pentamethyl-2,3-dihydrobenzofur an-5-yl)sulfonyl)-L-arginine
  MOD_RES 7: note = (S)-2-amino-4-(tert-butoxy)-4-oxobutanoic acid
  MOD_RES 10: note = N4-trityl-L-asparagine
  MOD_RES 11: note = 1-(tert-butoxycarbonyl)-L-tryptophan
  MOD_RES 14: note = N5-trityl-L-glutamine
  MOD_RES 15: note = O-(tert-butyl)-L-threonine
  MOD_RES 16: note = N6-(tert-butoxycarbonyl)-L-lysine
  MOD_RES 18: note = O-(tert-butyl)-L-threonine
  MOD_RES 19: note = (S)-2-amino-4-(tert-butoxy)-4-oxobutanoic acid
DNLAARDFINWLIQTKITD

SEQ ID NO: 90 - protein, 17 aa, synthetic construct
  REGION 1..17: note = synthetic peptide
HGDGSFSDEMNTILDNL

SEQ ID NO: 91 - protein, 16 aa, synthetic construct
  REGION 1..16: note = synthetic peptide
AARDFINWLIQTKITD

SEQ ID NO: 92 - protein, 17 aa, synthetic construct
  REGION 1..17: note = synthetic peptide
  MOD_RES 1: note = His has N-terminal and side chain protecting groups
  MOD_RES 3: note = Asp has side chain protecting group
  MOD_RES 5: note = Ser has side chain protecting group
  MOD_RES 7: note = Ser has side chain protecting group
  MOD_RES 8: note = Asp has side chain protecting group
  MOD_RES 9: note = Glu has side chain protecting group
  MOD_RES 11: note = Asn has side chain protecting group
  MOD_RES 12: note = Thr has side chain protecting group
  MOD_RES 15: note = Asp has side chain protecting group
  MOD_RES 16: note = Asn has side chain protecting group
  MOD_RES 17: note = Leu-OH or activated carboxylic ester of Leu
  VARIANT 17: note = Leu-OH or activated carboxylic ester of Leu
HGDGSFSDEMNTILDNL

SEQ ID NO: 93 - protein, 17 aa, synthetic construct
  REGION 1..17: note = synthetic peptide
  MOD_RES 1: note = N-alpha-(tert-butoxycarbonyl)-N-tau-trityl-L-histid ine
  MOD_RES 3: note = (S)-2-amino-4-(tert-butoxy)-4-oxobutanoic acid
  MOD_RES 5: note = O-(tert-butyl)-L-serine
  MOD_RES 7: note = O-(tert-butyl)-L-serine or Serine pseudoproline
  MOD_RES 8: note = (S)-2-amino-4-(tert-butoxy)-4-oxobutanoic acid
  MOD_RES 9: note = (S)-2-amino-5-(tert-butoxy)-5-oxopentanoic acid
  MOD_RES 11: note = N4-trityl-L-asparagine
  MOD_RES 12: note = O-(tert-butyl)-L-threonine
  MOD_RES 15: note = (S)-2-amino-4-(tert-butoxy)-4-oxobutanoic acid
  MOD_RES 16: note = N4-trityl-L-asparagine
  VARIANT 7: note = O-(tert-butyl)-L-serine or Serine pseudoproline
HGDGSFSDEMNTILDNL

SEQ ID NO: 94 - protein, 16 aa, synthetic construct
  REGION 1..16: note = synthetic peptide
  MOD_RES 3: note = Arg has side chain protecting group
  MOD_RES 4: note = Asp has side chain protecting group
  MOD_RES 7: note = Asn has side chain protecting group
  MOD_RES 8: note = Trp has side chain protecting group
  MOD_RES 11: note = Gln has side chain protecting group
  MOD_RES 12: note = Thr has side chain protecting group
  MOD_RES 13: note = Lys has side chain protecting group
  MOD_RES 15: note = Thr has side chain protecting group
  MOD_RES 16: note = Asp-OH or a carboxy protecting group
  VARIANT 16: note = Asp-OH or a carboxy protecting group
AARDFINWLIQTKITD

SEQ ID NO: 95 - protein, 16 aa, synthetic construct
  REGION 1..16: note = synthetic peptide
  MOD_RES 3: note = N-omega-((2,2,4,6,7-pentamethyl-2,3-dihydrobenzofur an-5-yl)sulfonyl)-L-arginine
  MOD_RES 4: note = (S)-2-amino-4-(tert-butoxy)-4-oxobutanoic acid
  MOD_RES 7: note = N4-trityl-L-asparagine
  MOD_RES 8: note = 1-(tert-butoxycarbonyl)-L-tryptophan
  MOD_RES 11: note = N5-trityl-L-glutamine
  MOD_RES 12: note = O-(tert-butyl)-L-threonine
  MOD_RES 13: note = N6-(tert-butoxycarbonyl)-L-lysine
  MOD_RES 15: note = O-(tert-butyl)-L-threonine
  MOD_RES 16: note = (S)-2-amino-4-(tert-butoxy)-4-oxobutanoic acid
AARDFINWLIQTKITD

SEQ ID NO: 96 - protein, 18 aa, synthetic construct
  REGION 1..18: note = synthetic peptide
HGDGSFSDEMNTILDNLA

SEQ ID NO: 97 - protein, 15 aa, synthetic construct
  REGION 1..15: note = synthetic peptide
ARDFINWLIQTKITD

SEQ ID NO: 98 - protein, 18 aa, synthetic construct
  REGION 1..18: note = synthetic peptide
  MOD_RES 1: note = His has N-terminal and side chain protecting groups
  MOD_RES 3: note = Asp has side chain protecting group
  MOD_RES 5: note = Ser has side chain protecting group
  MOD_RES 7: note = Ser has side chain protecting group
  MOD_RES 8: note = Asp has side chain protecting group
  MOD_RES 9: note = Glu has side chain protecting group
  MOD_RES 11: note = Asn has side chain protecting group
  MOD_RES 12: note = Thr has side chain protecting group
  MOD_RES 15: note = Asp has side chain protecting group
  MOD_RES 16: note = Asn has side chain protecting group
  MOD_RES 18: note = Ala-OH or activated carboxylic ester of Ala
  VARIANT 18: note = Ala-OH or activated carboxylic ester of Ala
HGDGSFSDEMNTILDNLA

SEQ ID NO: 99 - protein, 18 aa, synthetic construct
  REGION 1..18: note = synthetic peptide
  MOD_RES 1: note = N-alpha-(tert-butoxycarbonyl)-N-tau-trityl-L-histid ine
  MOD_RES 3: note = (S)-2-amino-4-(tert-butoxy)-4-oxobutanoic acid
  MOD_RES 5: note = O-(tert-butyl)-L-serine
  MOD_RES 7: note = O-(tert-butyl)-L-serine or Serine pseudoproline
  MOD_RES 8: note = (S)-2-amino-4-(tert-butoxy)-4-oxobutanoic acid
  MOD_RES 9: note = (S)-2-amino-5-(tert-butoxy)-5-oxopentanoic acid
  MOD_RES 11: note = N4-trityl-L-asparagine
  MOD_RES 12: note = O-(tert-butyl)-L-threonine
  MOD_RES 15: note = (S)-2-amino-4-(tert-butoxy)-4-oxobutanoic acid
  MOD_RES 16: note = N4-trityl-L-asparagine
  VARIANT 7: note = O-(tert-butyl)-L-serine or Serine pseudoproline
HGDGSFSDEMNTILDNLA

SEQ ID NO: 100 - protein, 15 aa, synthetic construct
  REGION 1..15: note = synthetic peptide
  MOD_RES 2: note = Arg has side chain protecting group
  MOD_RES 3: note = Asp has side chain protecting group
  MOD_RES 6: note = Asn has side chain protecting group
  MOD_RES 7: note = Trp has side chain protecting group
  MOD_RES 10: note = Gln has side chain protecting group
  MOD_RES 11: note = Thr has side chain protecting group
  MOD_RES 12: note = Lys has side chain protecting group
  MOD_RES 14: note = Thr has side chain protecting group
  MOD_RES 15: note = Asp-OH or a carboxy protecting group
  VARIANT 15: note = Asp-OH or a carboxy protecting group
ARDFINWLIQTKITD

SEQ ID NO: 101 - protein, 15 aa, synthetic construct
  REGION 1..15: note = synthetic peptide
  MOD_RES 2: note = N-omega-((2,2,4,6,7-pentamethyl-2,3-dihydrobenzofur an-5-yl)sulfonyl)-L-arginine
  MOD_RES 3: note = (S)-2-amino-4-(tert-butoxy)-4-oxobutanoic acid
  MOD_RES 6: note = N4-trityl-L-asparagine
  MOD_RES 7: note = 1-(tert-butoxycarbonyl)-L-tryptophan
  MOD_RES 10: note = N5-trityl-L-glutamine
  MOD_RES 11: note = O-(tert-butyl)-L-threonine
  MOD_RES 12: note = N6-(tert-butoxycarbonyl)-L-lysine
  MOD_RES 14: note = O-(tert-butyl)-L-threonine
  MOD_RES 15: note = (S)-2-amino-4-(tert-butoxy)-4-oxobutanoic acid
ARDFINWLIQTKITD

SEQ ID NO: 102 - protein, 19 aa, synthetic construct
  REGION 1..19: note = synthetic peptide
HGDGSFSDEMNTILDNLAA

SEQ ID NO: 103 - protein, 14 aa, synthetic construct
  REGION 1..14: note = synthetic peptide
RDFINWLIQTKITD

SEQ ID NO: 104 - protein, 19 aa, synthetic construct
  REGION 1..19: note = synthetic peptide
  MOD_RES 1: note = His has N-terminal and side chain protecting groups
  MOD_RES 3: note = Asp has side chain protecting group
  MOD_RES 5: note = Ser has side chain protecting group
  MOD_RES 7: note = Ser has side chain protecting group
  MOD_RES 8: note = Asp has side chain protecting group
  MOD_RES 9: note = Glu has side chain protecting group
  MOD_RES 11: note = Asn has side chain protecting group
  MOD_RES 12: note = Thr has side chain protecting group
  MOD_RES 15: note = Asp has side chain protecting group
  MOD_RES 16: note = Asn has side chain protecting group
  MOD_RES 19: note = Ala-OH or activated carboxylic ester of Ala
  VARIANT 19: note = Ala-OH or activated carboxylic ester of Ala
HGDGSFSDEMNTILDNLAA

SEQ ID NO: 105 - protein, 19 aa, synthetic construct
  REGION 1..19: note = synthetic peptide
  MOD_RES 1: note = N-alpha-(tert-butoxycarbonyl)-N-tau-trityl-L-histid ine
  MOD_RES 3: note = (S)-2-amino-4-(tert-butoxy)-4-oxobutanoic acid
  MOD_RES 5: note = O-(tert-butyl)-L-serine
  MOD_RES 7: note = O-(tert-butyl)-L-serine or Serine pseudoproline
  MOD_RES 8: note = (S)-2-amino-4-(tert-butoxy)-4-oxobutanoic acid
  MOD_RES 9: note = (S)-2-amino-5-(tert-butoxy)-5-oxopentanoic acid
  MOD_RES 11: note = N4-trityl-L-asparagine
  MOD_RES 12: note = O-(tert-butyl)-L-threonine
  MOD_RES 15: note = (S)-2-amino-4-(tert-butoxy)-4-oxobutanoic acid
  MOD_RES 16: note = N4-trityl-L-asparagine
  VARIANT 7: note = O-(tert-butyl)-L-serine or Serine pseudoproline
HGDGSFSDEMNTILDNLAA

SEQ ID NO: 106 - protein, 14 aa, synthetic construct
  REGION 1..14: note = synthetic peptide
  MOD_RES 1: note = Arg has side chain protecting group
  MOD_RES 2: note = Asp has side chain protecting group
  MOD_RES 5: note = Asn has side chain protecting group
  MOD_RES 6: note = Trp has side chain protecting group
  MOD_RES 9: note = Gln has side chain protecting group
  MOD_RES 10: note = Thr has side chain protecting group
  MOD_RES 11: note = Lys has side chain protecting group
  MOD_RES 13: note = Thr has side chain protecting group
  MOD_RES 14: note = Asp-OH or a carboxy protecting group
  VARIANT 14: note = Asp-OH or a carboxy protecting group
RDFINWLIQTKITD

SEQ ID NO: 107 - protein, 14 aa, synthetic construct
  REGION 1..14: note = synthetic peptide
  MOD_RES 1: note = N-omega-((2,2,4,6,7-pentamethyl-2,3-dihydrobenzofur an-5-yl)sulfonyl)-L-arginine
  MOD_RES 2: note = (S)-2-amino-4-(tert-butoxy)-4-oxobutanoic acid
  MOD_RES 5: note = N4-trityl-L-asparagine
  MOD_RES 6: note = 1-(tert-butoxycarbonyl)-L-tryptophan
  MOD_RES 9: note = N5-trityl-L-glutamine
  MOD_RES 10: note = O-(tert-butyl)-L-threonine
  MOD_RES 11: note = N6-(tert-butoxycarbonyl)-L-lysine
  MOD_RES 13: note = O-(tert-butyl)-L-threonine
  MOD_RES 14: note = (S)-2-amino-4-(tert-butoxy)-4-oxobutanoic acid
RDFINWLIQTKITD

SEQ ID NO: 108 - protein, 33 aa, synthetic construct
  REGION 1..33: note = GLP-2 peptide
  MOD_RES 2: note = Aib
HXDGSFSDEMNTILDNLAARDFINWLIQTKITD

SEQ ID NO: 109 - protein, 33 aa, synthetic construct
  REGION 1..33: note = GLP-2 peptide
HADGSFSDEMNTILDNLAARDFINWLIQTKITD

SEQ ID NO: 110 - protein, 30 aa, synthetic construct
  REGION 1..30: note = GLP-1 peptide
HAEGTFTSDVSSYLEGQAAKEFIAWLVKGR

SEQ ID NO: 111 - protein, 19 aa, synthetic construct
  REGION 1..19: note = Liraglutide (GLP-1 analogue) sequence
HAEGTFTSDVSSYLEGQAA

SEQ ID NO: 112 - protein, 11 aa, synthetic construct
  REGION 1..11: note = Liraglutide (GLP-1 analogue) sequence
EFIAWLVRGRG

SEQ ID NO: 113 - protein, 19 aa, synthetic construct
  REGION 1..19: note = Semaglutide (GLP-1 analogue) sequence
  MOD_RES 2: note = Aib
HXEGTFTSDVSSYLEGQAA

SEQ ID NO: 114 - protein, 14 aa, synthetic construct
  REGION 1..14: note = synthetic peptide
  MOD_RES 1: note = N-alpha-(tert-butoxycarbonyl)-N-tau-trityl-L-histid ine
  MOD_RES 2: note = Aib
  MOD_RES 3: note = (S)-2-amino-4-(tert-butoxy)-4-oxobutanoic acid
  MOD_RES 5: note = O-(tert-butyl)-L-serine
  MOD_RES 7: note = O-(tert-butyl)-L-serine or Serine pseudoproline
  MOD_RES 8: note = (S)-2-amino-4-(tert-butoxy)-4-oxobutanoic acid
  MOD_RES 9: note = (S)-2-amino-5-(tert-butoxy)-5-oxopentanoic acid
  MOD_RES 11: note = N4-trityl-L-asparagine
  MOD_RES 12: note = O-(tert-butyl)-L-threonine
  VARIANT 7: note = O-(tert-butyl)-L-serine or Serine pseudoproline
HXDGSFSDEMNTIL

SEQ ID NO: 115 - protein, 19 aa, synthetic construct
  REGION 1..19: note = synthetic peptide
  MOD_RES 1: note = (S)-2-amino-4-(tert-butoxy)-4-oxobutanoic acid
  MOD_RES 2: note = N4-trityl-L-asparagine
  MOD_RES 6: note = N-omega-((2,2,4,6,7-pentamethyl-2,3-dihydrobenzofur an-5-yl)sulfonyl)-L-arginine
  MOD_RES 7: note = (S)-2-amino-4-(tert-butoxy)-4-oxobutanoic acid
  MOD_RES 10: note = N4-trityl-L-asparagine
  MOD_RES 11: note = 1-(tert-butoxycarbonyl)-L-tryptophan
  MOD_RES 14: note = N5-trityl-L-glutamine
  MOD_RES 15: note = O-(tert-butyl)-L-threonine or Threonine pseudoproline
  MOD_RES 16: note = N6-(tert-butoxycarbonyl)-L-lysine
  MOD_RES 18: note = O-(tert-butyl)-L-threonine
  MOD_RES 19: note = (S)-2-amino-4-(tert-butoxy)-4-oxobutanoic acid and Asp-OH or a carboxy protecting group
  VARIANT 15: note = O-(tert-butyl)-L-threonine or Threonine pseudoproline
  VARIANT 19: note = (S)-2-amino-4-(tert-butoxy)-4-oxobutanoic acid and Asp-OH or a carboxy protecting group
DNLAARDFINWLIQTKITD

SEQ ID NO: 116 - protein, 17 aa, synthetic construct
  REGION 1..17: note = synthetic peptide
  MOD_RES 1: note = N-alpha-(tert-butoxycarbonyl)-N-tau-trityl-L-histid ine
  MOD_RES 2: note = Aib
  MOD_RES 3: note = (S)-2-amino-4-(tert-butoxy)-4-oxobutanoic acid
  MOD_RES 5: note = O-(tert-butyl)-L-serine
  MOD_RES 7: note = O-(tert-butyl)-L-serine or Serine pseudoproline
  MOD_RES 8: note = (S)-2-amino-4-(tert-butoxy)-4-oxobutanoic acid
  MOD_RES 9: note = (S)-2-amino-5-(tert-butoxy)-5-oxopentanoic acid
  MOD_RES 11: note = N4-trityl-L-asparagine
  MOD_RES 12: note = O-(tert-butyl)-L-threonine
  MOD_RES 15: note = (S)-2-amino-4-(tert-butoxy)-4-oxobutanoic acid
  MOD_RES 16: note = N4-trityl-L-asparagine
  VARIANT 7: note = O-(tert-butyl)-L-serine or Serine pseudoproline
HXDGSFSDEMNTILDNL

SEQ ID NO: 117 - protein, 16 aa, synthetic construct
  REGION 1..16: note = synthetic peptide
  MOD_RES 3: note = N-omega-((2,2,4,6,7-pentamethyl-2,3-dihydrobenzofur an-5-yl)sulfonyl)-L-arginine
  MOD_RES 4: note = (S)-2-amino-4-(tert-butoxy)-4-oxobutanoic acid
  MOD_RES 7: note = N4-trityl-L-asparagine
  MOD_RES 8: note = 1-(tert-butoxycarbonyl)-L-tryptophan
  MOD_RES 11: note = N5-trityl-L-glutamine
  MOD_RES 12: note = O-(tert-butyl)-L-threonine or Threonine pseudoproline
  MOD_RES 13: note = N6-(tert-butoxycarbonyl)-L-lysine
  MOD_RES 15: note = O-(tert-butyl)-L-threonine
  MOD_RES 16: note = (S)-2-amino-4-(tert-butoxy)-4-oxobutanoic acid and Asp-OH or a carboxy protecting group
  VARIANT 12: note = O-(tert-butyl)-L-threonine or Threonine pseudoproline
  VARIANT 16: note = (S)-2-amino-4-(tert-butoxy)-4-oxobutanoic acid and Asp-OH or a carboxy protecting group
AARDFINWLIQTKITD

SEQ ID NO: 118 - protein, 18 aa, synthetic construct
  REGION 1..18: note = synthetic peptide
  MOD_RES 1: note = N-alpha-(tert-butoxycarbonyl)-N-tau-trityl-L-histid ine
  MOD_RES 2: note = Aib
  MOD_RES 3: note = (S)-2-amino-4-(tert-butoxy)-4-oxobutanoic acid
  MOD_RES 5: note = O-(tert-butyl)-L-serine
  MOD_RES 7: note = O-(tert-butyl)-L-serine or Serine pseudoproline
  MOD_RES 8: note = (S)-2-amino-4-(tert-butoxy)-4-oxobutanoic acid
  MOD_RES 9: note = (S)-2-amino-5-(tert-butoxy)-5-oxopentanoic acid
  MOD_RES 11: note = N4-trityl-L-asparagine
  MOD_RES 12: note = O-(tert-butyl)-L-threonine
  MOD_RES 15: note = (S)-2-amino-4-(tert-butoxy)-4-oxobutanoic acid
  MOD_RES 16: note = N4-trityl-L-asparagine
  VARIANT 7: note = O-(tert-butyl)-L-serine or Serine pseudoproline
HXDGSFSDEMNTILDNLA

SEQ ID NO: 119 - protein, 15 aa, synthetic construct
  REGION 1..15: note = synthetic peptide
  MOD_RES 2: note = N-omega-((2,2,4,6,7-pentamethyl-2,3-dihydrobenzofur an-5-yl)sulfonyl)-L-arginine
  MOD_RES 3: note = (S)-2-amino-4-(tert-butoxy)-4-oxobutanoic acid
  MOD_RES 6: note = N4-trityl-L-asparagine
  MOD_RES 7: note = 1-(tert-butoxycarbonyl)-L-tryptophan
  MOD_RES 10: note = N5-trityl-L-glutamine
  MOD_RES 11: note = O-(tert-butyl)-L-threonine or Threonine pseudoproline
  MOD_RES 12: note = N6-(tert-butoxycarbonyl)-L-lysine
  MOD_RES 14: note = O-(tert-butyl)-L-threonine
  VARIANT 11: note = O-(tert-butyl)-L-threonine or Threonine pseudoproline
ARDFINWLIQTKITD

SEQ ID NO: 120 - protein, 19 aa, synthetic construct
  REGION 1..19: note = synthetic peptide
  MOD_RES 1: note = N-alpha-(tert-butoxycarbonyl)-N-tau-trityl-L-histid ine
  MOD_RES 2: note = Aib
  MOD_RES 3: note = (S)-2-amino-4-(tert-butoxy)-4-oxobutanoic acid
  MOD_RES 5: note = O-(tert-butyl)-L-serine
  MOD_RES 7: note = O-(tert-butyl)-L-serine or Serine pseudoproline
  MOD_RES 8: note = (S)-2-amino-4-(tert-butoxy)-4-oxobutanoic acid
  MOD_RES 9: note = (S)-2-amino-5-(tert-butoxy)-5-oxopentanoic acid
  MOD_RES 11: note = N4-trityl-L-asparagine
  MOD_RES 12: note = O-(tert-butyl)-L-threonine
  MOD_RES 15: note = (S)-2-amino-4-(tert-butoxy)-4-oxobutanoic acid
  MOD_RES 16: note = N4-trityl-L-asparagine
  VARIANT 7: note = O-(tert-butyl)-L-serine or Serine pseudoproline
HXDGSFSDEMNTILDNLAA

SEQ ID NO: 121 - protein, 14 aa, synthetic construct
  REGION 1..14: note = synthetic peptide
  MOD_RES 1: note = N-omega-((2,2,4,6,7-pentamethyl-2,3-dihydrobenzofur an-5-yl)sulfonyl)-L-arginine
  MOD_RES 2: note = (S)-2-amino-4-(tert-butoxy)-4-oxobutanoic acid
  MOD_RES 5: note = N4-trityl-L-asparagine
  MOD_RES 6: note = 1-(tert-butoxycarbonyl)-L-tryptophan
  MOD_RES 9: note = N5-trityl-L-glutamine
  MOD_RES 10: note = O-(tert-butyl)-L-threonine or Threonine pseudoproline
  MOD_RES 11: note = N6-(tert-butoxycarbonyl)-L-lysine
  MOD_RES 13: note = O-(tert-butyl)-L-threonine
  MOD_RES 14: note = (S)-2-amino-4-(tert-butoxy)-4-oxobutanoic acid and Asp-OH or a carboxy protecting group
  VARIANT 10: note = O-(tert-butyl)-L-threonine or Threonine pseudoproline
  VARIANT 14: note = (S)-2-amino-4-(tert-butoxy)-4-oxobutanoic acid and Asp-OH or a carboxy protecting group
RDFINWLIQTKITD

SEQ ID NO: 122 - protein, 4 aa, synthetic construct
  REGION 1..4: note = synthetic peptide
  MOD_RES 1: note = N-alpha-(tert-butoxycarbonyl)-N-tau-trityl-L-histid ine
  MOD_RES 3: note = (S)-2-amino-4-(tert-butoxy)-4-oxobutanoic acid
HGDG

SEQ ID NO: 123 - skipped

SEQ ID NO: 124 - protein, 4 aa, synthetic construct
  REGION 1..4: note = synthetic peptide
  MOD_RES 1: note = N-alpha-(tert-butoxycarbonyl)-N-tau-trityl-L-histid ine
  MOD_RES 3: note = (S)-2-amino-4-(tert-butoxy)-4-oxobutanoic acid
HGDG

SEQ ID NO: 125 - skipped

SEQ ID NO: 126 - protein, 10 aa, synthetic construct
  REGION 1..10: note = synthetic peptide
  MOD_RES 1: note = O-(tert-butyl)-L-serine
  MOD_RES 3: note = O-(tert-butyl)-L-serine
  MOD_RES 4: note = (S)-2-amino-4-(tert-butoxy)-4-oxobutanoic acid
  MOD_RES 5: note = (S)-2-amino-5-(tert-butoxy)-5-oxopentanoic acid
  MOD_RES 7: note = N4-trityl-L-asparagine
  MOD_RES 8: note = O-(tert-butyl)-L-threonine
  MOD_RES 10: note = Leu-O-2-chlorotrityl resin or Leu-OH
  VARIANT 10: note = Leu-O-2-chlorotrityl resin or Leu-OH
SFSDEMNTIL

SEQ ID NO: 127 - protein, 13 aa, synthetic construct
  REGION 1..13: note = synthetic peptide
  MOD_RES 1: note = O-(tert-butyl)-L-serine
  MOD_RES 3: note = O-(tert-butyl)-L-serine
  MOD_RES 4: note = (S)-2-amino-4-(tert-butoxy)-4-oxobutanoic acid
  MOD_RES 5: note = (S)-2-amino-5-(tert-butoxy)-5-oxopentanoic acid
  MOD_RES 7: note = N4-trityl-L-asparagine
  MOD_RES 8: note = O-(tert-butyl)-L-threonine
  MOD_RES 11: note = (S)-2-amino-4-(tert-butoxy)-4-oxobutanoic acid
  MOD_RES 12: note = N4-trityl-L-asparagine
  MOD_RES 13: note = Leu-O-2-chlorotrityl resin or Leu-OH
  VARIANT 13: note = Leu-O-2-chlorotrityl resin or Leu-OH
SFSDEMNTILDNL

SEQ ID NO: 128 - protein, 14 aa, synthetic construct
  REGION 1..14: note = synthetic peptide
  MOD_RES 1: note = O-(tert-butyl)-L-serine
  MOD_RES 3: note = O-(tert-butyl)-L-serine
  MOD_RES 4: note = (S)-2-amino-4-(tert-butoxy)-4-oxobutanoic acid
  MOD_RES 5: note = (S)-2-amino-5-(tert-butoxy)-5-oxopentanoic acid
  MOD_RES 7: note = N4-trityl-L-asparagine
  MOD_RES 8: note = O-(tert-butyl)-L-threonine
  MOD_RES 11: note = (S)-2-amino-4-(tert-butoxy)-4-oxobutanoic acid
  MOD_RES 12: note = N4-trityl-L-asparagine
  MOD_RES 14: note = Ala-O-2-chlorotrityl resin or Ala-OH
  VARIANT 14: note = Ala-O-2-chlorotrityl resin or Ala-OH
SFSDEMNTILDNLA

SEQ ID NO: 129 - protein, 15 aa, synthetic construct
  REGION 1..15: note = synthetic peptide
  MOD_RES 1: note = O-(tert-butyl)-L-serine
  MOD_RES 3: note = O-(tert-butyl)-L-serine
  MOD_RES 4: note = (S)-2-amino-4-(tert-butoxy)-4-oxobutanoic acid
  MOD_RES 5: note = (S)-2-amino-5-(tert-butoxy)-5-oxopentanoic acid
  MOD_RES 7: note = N4-trityl-L-asparagine
  MOD_RES 8: note = O-(tert-butyl)-L-threonine
  MOD_RES 11: note = (S)-2-amino-4-(tert-butoxy)-4-oxobutanoic acid
  MOD_RES 12: note = N4-trityl-L-asparagine
  MOD_RES 15: note = Ala-O-2-chlorotrityl resin or Ala-OH
  VARIANT 15: note = Ala-O-2-chlorotrityl resin or Ala-OH
SFSDEMNTILDNLAA

SEQ ID NO: 130 - protein, 14 aa, synthetic construct
  REGION 1..14: note = synthetic peptide
  MOD_RES 1: note = N-alpha-(tert-butoxycarbonyl)-N-tau-trityl-L-histid ine
  MOD_RES 3: note = (S)-2-amino-4-(tert-butoxy)-4-oxobutanoic acid
  MOD_RES 5: note = O-(tert-butyl)-L-serine
  MOD_RES 7: note = O-(tert-butyl)-L-serine or Serine pseudoproline
  MOD_RES 8: note = (S)-2-amino-4-(tert-butoxy)-4-oxobutanoic acid
  MOD_RES 9: note = (S)-2-amino-5-(tert-butoxy)-5-oxopentanoic acid
  MOD_RES 11: note = N4-trityl-L-asparagine
  MOD_RES 12: note = O-(tert-butyl)-L-threonine
  VARIANT 7: note = O-(tert-butyl)-L-serine or Serine pseudoproline
HGDGSFSDEMNTIL

SEQ ID NO: 131 - protein, 17 aa, synthetic construct
  REGION 1..17: note = synthetic peptide
  MOD_RES 1: note = N-alpha-(tert-butoxycarbonyl)-N-tau-trityl-L-histid ine
  MOD_RES 3: note = (S)-2-amino-4-(tert-butoxy)-4-oxobutanoic acid
  MOD_RES 5: note = O-(tert-butyl)-L-serine
  MOD_RES 7: note = O-(tert-butyl)-L-serine or Serine pseudoproline
  MOD_RES 8: note = (S)-2-amino-4-(tert-butoxy)-4-oxobutanoic acid
  MOD_RES 9: note = (S)-2-amino-5-(tert-butoxy)-5-oxopentanoic acid
  MOD_RES 11: note = N4-trityl-L-asparagine
  MOD_RES 12: note = O-(tert-butyl)-L-threonine
  MOD_RES 15: note = (S)-2-amino-4-(tert-butoxy)-4-oxobutanoic acid
  MOD_RES 16: note = N4-trityl-L-asparagine
  VARIANT 7: note = O-(tert-butyl)-L-serine or Serine pseudoproline
HGDGSFSDEMNTILDNL

SEQ ID NO: 132 - protein, 18 aa, synthetic construct
  REGION 1..18: note = synthetic peptide
  MOD_RES 1: note = N-alpha-(tert-butoxycarbonyl)-N-tau-trityl-L-histid ine
  MOD_RES 3: note = (S)-2-amino-4-(tert-butoxy)-4-oxobutanoic acid
  MOD_RES 5: note = O-(tert-butyl)-L-serine
  MOD_RES 7: note = O-(tert-butyl)-L-serine or Serine pseudoproline
  MOD_RES 8: note = (S)-2-amino-4-(tert-butoxy)-4-oxobutanoic acid
  MOD_RES 9: note = (S)-2-amino-5-(tert-butoxy)-5-oxopentanoic acid
  MOD_RES 11: note = N4-trityl-L-asparagine
  MOD_RES 12: note = O-(tert-butyl)-L-threonine
  MOD_RES 15: note = (S)-2-amino-4-(tert-butoxy)-4-oxobutanoic acid
  MOD_RES 16: note = N4-trityl-L-asparagine
  VARIANT 7: note = O-(tert-butyl)-L-serine or Serine pseudoproline
HGDGSFSDEMNTILDNLA

SEQ ID NO: 133 - protein, 19 aa, synthetic construct
  REGION 1..19: note = synthetic peptide
  MOD_RES 1: note = N-alpha-(tert-butoxycarbonyl)-N-tau-trityl-L-histid ine
  MOD_RES 3: note = (S)-2-amino-4-(tert-butoxy)-4-oxobutanoic acid
  MOD_RES 5: note = O-(tert-butyl)-L-serine
  MOD_RES 7: note = O-(tert-butyl)-L-serine or Serine pseudoproline
  MOD_RES 8: note = (S)-2-amino-4-(tert-butoxy)-4-oxobutanoic acid
  MOD_RES 9: note = (S)-2-amino-5-(tert-butoxy)-5-oxopentanoic acid
  MOD_RES 11: note = N4-trityl-L-asparagine
  MOD_RES 12: note = O-(tert-butyl)-L-threonine
  MOD_RES 15: note = (S)-2-amino-4-(tert-butoxy)-4-oxobutanoic acid
  MOD_RES 16: note = N4-trityl-L-asparagine
  VARIANT 7: note = O-(tert-butyl)-L-serine or Serine pseudoproline
HGDGSFSDEMNTILDNLAA

SEQ ID NO: 134 - protein, 14 aa, synthetic construct
  REGION 1..14: note = synthetic peptide
  MOD_RES 1: note = N-alpha-(tert-butoxycarbonyl)-N-tau-trityl-L-histid ine
  MOD_RES 2: note = Aib
  MOD_RES 3: note = (S)-2-amino-4-(tert-butoxy)-4-oxobutanoic acid
  MOD_RES 5: note = O-(tert-butyl)-L-serine
  MOD_RES 7: note = O-(tert-butyl)-L-serine or Serine pseudoproline
  MOD_RES 8: note = (S)-2-amino-4-(tert-butoxy)-4-oxobutanoic acid
  MOD_RES 9: note = (S)-2-amino-5-(tert-butoxy)-5-oxopentanoic acid
  MOD_RES 11: note = N4-trityl-L-asparagine
  MOD_RES 12: note = O-(tert-butyl)-L-threonine
  VARIANT 7: note = O-(tert-butyl)-L-serine or Serine pseudoproline
HXDGSFSDEMNTIL

SEQ ID NO: 135 - protein, 17 aa, synthetic construct
  REGION 1..17: note = synthetic peptide
  MOD_RES 1: note = N-alpha-(tert-butoxycarbonyl)-N-tau-trityl-L-histid ine
  MOD_RES 2: note = Aib
  MOD_RES 3: note = (S)-2-amino-4-(tert-butoxy)-4-oxobutanoic acid
  MOD_RES 5: note = O-(tert-butyl)-L-serine
  MOD_RES 7: note = O-(tert-butyl)-L-serine or Serine pseudoproline
  MOD_RES 8: note = (S)-2-amino-4-(tert-butoxy)-4-oxobutanoic acid
  MOD_RES 9: note = (S)-2-amino-5-(tert-butoxy)-5-oxopentanoic acid
  MOD_RES 11: note = N4-trityl-L-asparagine
  MOD_RES 12: note = O-(tert-butyl)-L-threonine
  MOD_RES 15: note = (S)-2-amino-4-(tert-butoxy)-4-oxobutanoic acid
  MOD_RES 16: note = N4-trityl-L-asparagine
  VARIANT 7: note = O-(tert-butyl)-L-serine or Serine pseudoproline
HXDGSFSDEMNTILDNL

SEQ ID NO: 136 - protein, 18 aa, synthetic construct
  REGION 1..18: note = synthetic peptide
  MOD_RES 1: note = N-alpha-(tert-butoxycarbonyl)-N-tau-trityl-L-histid ine
  MOD_RES 2: note = Aib
  MOD_RES 3: note = (S)-2-amino-4-(tert-butoxy)-4-oxobutanoic acid
  MOD_RES 5: note = O-(tert-butyl)-L-serine
  MOD_RES 7: note = O-(tert-butyl)-L-serine or Serine pseudoproline
  MOD_RES 8: note = (S)-2-amino-4-(tert-butoxy)-4-oxobutanoic acid
  MOD_RES 9: note = (S)-2-amino-5-(tert-butoxy)-5-oxopentanoic acid
  MOD_RES 11: note = N4-trityl-L-asparagine
  MOD_RES 12: note = O-(tert-butyl)-L-threonine
  MOD_RES 15: note = (S)-2-amino-4-(tert-butoxy)-4-oxobutanoic acid
  MOD_RES 16: note = N4-trityl-L-asparagine
  VARIANT 7: note = O-(tert-butyl)-L-serine or Serine pseudoproline
HXDGSFSDEMNTILDNLA

SEQ ID NO: 137 - protein, 19 aa, synthetic construct
  REGION 1..19: note = synthetic peptide
  MOD_RES 1: note = N-alpha-(tert-butoxycarbonyl)-N-tau-trityl-L-histid ine
  MOD_RES 2: note = Aib
  MOD_RES 3: note = (S)-2-amino-4-(tert-butoxy)-4-oxobutanoic acid
  MOD_RES 5: note = O-(tert-butyl)-L-serine
  MOD_RES 7: note = O-(tert-butyl)-L-serine or Serine pseudoproline
  MOD_RES 8: note = (S)-2-amino-4-(tert-butoxy)-4-oxobutanoic acid
  MOD_RES 9: note = (S)-2-amino-5-(tert-butoxy)-5-oxopentanoic acid
  MOD_RES 11: note = N4-trityl-L-asparagine
  MOD_RES 12: note = O-(tert-butyl)-L-threonine
  MOD_RES 15: note = (S)-2-amino-4-(tert-butoxy)-4-oxobutanoic acid
  MOD_RES 16: note = N4-trityl-L-asparagine
  VARIANT 7: note = O-(tert-butyl)-L-serine or Serine pseudoproline
HXDGSFSDEMNTILDNLAA

SEQ ID NO: 138 - protein, 19 aa, synthetic construct
  REGION 1..19: note = synthetic peptide
  MOD_RES 1: note = (S)-2-amino-4-(tert-butoxy)-4-oxobutanoic acid
  MOD_RES 2: note = N4-trityl-L-asparagine
  MOD_RES 6: note = N-omega-((2,2,4,6,7-pentamethyl-2,3-dihydrobenzofur an-5-yl)sulfonyl)-L-arginine
  MOD_RES 7: note = (S)-2-amino-4-(tert-butoxy)-4-oxobutanoic acid
  MOD_RES 10: note = N4-trityl-L-asparagine
  MOD_RES 11: note = 1-(tert-butoxycarbonyl)-L-tryptophan
  MOD_RES 14: note = N5-trityl-L-glutamine
  MOD_RES 15: note = O-(tert-butyl)-L-threonine or Threonine pseudoproline
  MOD_RES 16: note = N6-(tert-butoxycarbonyl)-L-lysine
  MOD_RES 18: note = O-(tert-butyl)-L-threonine
  MOD_RES 19: note = (S)-2-amino-4-(tert-butoxy)-4-oxobutanoic acid and Asp-OH or a carboxy protecting group
  VARIANT 15: note = O-(tert-butyl)-L-threonine or Threonine pseudoproline
  VARIANT 19: note = (S)-2-amino-4-(tert-butoxy)-4-oxobutanoic acid and Asp-OH or a carboxy protecting group
DNLAARDFINWLIQTKITD

SEQ ID NO: 139 - protein, 16 aa, synthetic construct
  REGION 1..16: note = synthetic peptide
  MOD_RES 3: note = N-omega-((2,2,4,6,7-pentamethyl-2,3-dihydrobenzofur an-5-yl)sulfonyl)-L-arginine
  MOD_RES 4: note = (S)-2-amino-4-(tert-butoxy)-4-oxobutanoic acid
  MOD_RES 7: note = N4-trityl-L-asparagine
  MOD_RES 8: note = 1-(tert-butoxycarbonyl)-L-tryptophan
  MOD_RES 11: note = N5-trityl-L-glutamine
  MOD_RES 12: note = O-(tert-butyl)-L-threonine or Threonine pseudoproline
  MOD_RES 13: note = N6-(tert-butoxycarbonyl)-L-lysine
  MOD_RES 15: note = O-(tert-butyl)-L-threonine
  MOD_RES 16: note = (S)-2-amino-4-(tert-butoxy)-4-oxobutanoic acid and Asp-O-2-chlorotrityl resin or Asp-OH
  VARIANT 12: note = O-(tert-butyl)-L-threonine or Threonine pseudoproline
  VARIANT 16: note = (S)-2-amino-4-(tert-butoxy)-4-oxobutanoic acid and Asp-O-2-chlorotrityl resin or Asp-OH
AARDFINWLIQTKITD

SEQ ID NO: 140 - protein, 15 aa, synthetic construct
  REGION 1..15: note = synthetic peptide
  MOD_RES 2: note = N-omega-((2,2,4,6,7-pentamethyl-2,3-dihydrobenzofur an-5-yl)sulfonyl)-L-arginine
  MOD_RES 3: note = (S)-2-amino-4-(tert-butoxy)-4-oxobutanoic acid
  MOD_RES 6: note = N4-trityl-L-asparagine
  MOD_RES 7: note = 1-(tert-butoxycarbonyl)-L-tryptophan
  MOD_RES 10: note = N5-trityl-L-glutamine
  MOD_RES 11: note = O-(tert-butyl)-L-threonine or Threonine pseudoproline
  MOD_RES 12: note = N6-(tert-butoxycarbonyl)-L-lysine
  MOD_RES 14: note = O-(tert-butyl)-L-threonine
  MOD_RES 15: note = (S)-2-amino-4-(tert-butoxy)-4-oxobutanoic acid and Asp-O-2-chlorotrityl resin or Asp-OH
  VARIANT 11: note = O-(tert-butyl)-L-threonine or Threonine pseudoproline
  VARIANT 15: note = (S)-2-amino-4-(tert-butoxy)-4-oxobutanoic acid and Asp-O-2-chlorotrityl resin or Asp-OH
ARDFINWLIQTKITD

SEQ ID NO: 141 - protein, 14 aa, synthetic construct
  REGION 1..14: note = synthetic peptide
  MOD_RES 1: note = N-omega-((2,2,4,6,7-pentamethyl-2,3-dihydrobenzofur an-5-yl)sulfonyl)-L-arginine
  MOD_RES 2: note = (S)-2-amino-4-(tert-butoxy)-4-oxobutanoic acid
  MOD_RES 5: note = N4-trityl-L-asparagine
  MOD_RES 6: note = 1-(tert-butoxycarbonyl)-L-tryptophan
  MOD_RES 9: note = N5-trityl-L-glutamine
  MOD_RES 10: note = O-(tert-butyl)-L-threonine or Threonine pseudoproline
  MOD_RES 11: note = N6-(tert-butoxycarbonyl)-L-lysine
  MOD_RES 13: note = O-(tert-butyl)-L-threonine
  MOD_RES 14: note = (S)-2-amino-4-(tert-butoxy)-4-oxobutanoic acid and Asp-O-2-chlorotrityl resin or Asp-OH
  VARIANT 10: note = O-(tert-butyl)-L-threonine or Threonine pseudoproline
  VARIANT 14: note = (S)-2-amino-4-(tert-butoxy)-4-oxobutanoic acid and Asp-O-2-chlorotrityl resin or Asp-OH
RDFINWLIQTKITD

SEQ ID NO: 142 - protein, 10 aa, synthetic construct
  REGION 1..10: note = synthetic peptide
  MOD_RES 1: note = N6-(4-carboxy-4-palmitamidobutanoyl)lysine
  MOD_RES 2: note = (S)-2-amino-5-(tert-butoxy)-5-oxopentanoic acid
  MOD_RES 6: note = 1-(tert-butoxycarbonyl)-L-tryptophan
  MOD_RES 9: note = N-omega-((2,2,4,6,7-pentamethyl-2,3-dihydrobenzofur an-5-yl)sulfonyl)-L-arginine
KEFIAWLVRG

SEQ ID NO: 143 - protein, 10 aa, synthetic construct
  REGION 1..10: note = synthetic peptide
  MOD_RES 1: note = Fmoc-Lys(N-(17-carboxy-1-oxoheptadecyl)-L-gamma-glu tamyl-2-[2-(2- aminoethoxy)ethoxy]acetyl-2-[2-(2-aminoetho xy)ethoxy]acetyl)
  MOD_RES 2: note = (S)-2-amino-5-(tert-butoxy)-5-oxopentanoic acid
  MOD_RES 6: note = 1-(tert-butoxycarbonyl)-L-tryptophan
  MOD_RES 9: note = N-omega-((2,2,4,6,7-pentamethyl-2,3-dihydrobenzofur an-5-yl)sulfonyl)-L-arginine
KEFIAWLVRG

SEQ ID NO: 144 - protein, 31 aa, synthetic construct; identical to SEQ ID NO: 1
  REGION 1..31: note = synthetic peptide
  MOD_RES 1: note = N-alpha-(tert-butoxycarbonyl)-N-tau-trityl-L-histid ine
  MOD_RES 3: note = (S)-2-amino-5-(tert-butoxy)-5-oxopentanoic acid
  MOD_RES 5: note = O-(tert-butyl)-L-threonine
  MOD_RES 7: note = O-(tert-butyl)-L-threonine
  MOD_RES 8: note = O-(tert-butyl)-L-serine
  MOD_RES 9: note = (S)-2-amino-4-(tert-butoxy)-4-oxobutanoic acid
  MOD_RES 11: note = O-(tert-butyl)-L-serine
  MOD_RES 12: note = O-(tert-butyl)-L-serine
  MOD_RES 13: note = (S)-2-amino-3-(4-(tert-butoxy)phenyl)propanoic acid
  MOD_RES 15: note = (S)-2-amino-5-(tert-butoxy)-5-oxopentanoic acid
  MOD_RES 17: note = N5-trityl-L-glutamine
  MOD_RES 20: note = Lys(N-(17-carboxy-1-oxoheptadecyl)-L-gamma-glutamyl -2-[2-(2-amino ethoxy)ethoxy]acetyl-2-[2-(2-aminoethoxy)ethoxy]acetyl)
  MOD_RES 21: note = (S)-2-amino-5-(tert-butoxy)-5-oxopentanoic acid
  MOD_RES 25: note = 1-(tert-butoxycarbonyl)-L-tryptophan
  MOD_RES 28: note = N-omega-((2,2,4,6,7-pentamethyl-2,3-dihydrobenzofur an-5-yl)sulfonyl)-L-arginine
  MOD_RES 30: note = N-omega-((2,2,4,6,7-pentamethyl-2,3-dihydrobenzofur an-5-yl)sulfonyl)-L-arginine

SEQ ID NO: 145 - protein, 4 aa, synthetic construct
  REGION 1..4: note = synthetic peptide
  MOD_RES 1: note = N-alpha-(tert-butoxycarbonyl)-N-tau-trityl-L-histid ine
  MOD_RES 3: note = (S)-2-amino-5-(tert-butoxy)-5-oxopentanoic acid
HAEG

SEQ ID NO: 146 - skipped

SEQ ID NO: 147 - protein, 19 aa, synthetic construct
  REGION 1..19: note = synthetic peptide
  MOD_RES 1: note = N-alpha-(tert-butoxycarbonyl)-N-tau-trityl-L-histid ine
  MOD_RES 3: note = (S)-2-amino-5-(tert-butoxy)-5-oxopentanoic acid
  MOD_RES 5: note = O-(tert-butyl)-L-threonine
  MOD_RES 7: note = O-(tert-butyl)-L-threonine
  MOD_RES 8: note = O-(tert-butyl)-L-serine
  MOD_RES 9: note = (S)-2-amino-4-(tert-butoxy)-4-oxobutanoic acid
  MOD_RES 11: note = O-(tert-butyl)-L-serine
  MOD_RES 12: note = O-(tert-butyl)-L-serine
  MOD_RES 13: note = (S)-2-amino-3-(4-(tert-butoxy)phenyl)propanoic acid
  MOD_RES 15: note = (S)-2-amino-5-(tert-butoxy)-5-oxopentanoic acid
  MOD_RES 17: note = N5-trityl-L-glutamine
HAEGTFTSDVSSYLEGQAA

SEQ ID NO: 148 - protein, 19 aa, synthetic construct
  REGION 1..19: note = synthetic peptide
  MOD_RES 1: note = N-alpha-(tert-butoxycarbonyl)-N-tau-trityl-L-histid ine
  MOD_RES 2: note = Aib
  MOD_RES 3: note = (S)-2-amino-5-(tert-butoxy)-5-oxopentanoic acid
  MOD_RES 5: note = O-(tert-butyl)-L-threonine
  MOD_RES 7: note = O-(tert-butyl)-L-threonine
  MOD_RES 8: note = O-(tert-butyl)-L-serine
  MOD_RES 9: note = (S)-2-amino-4-(tert-butoxy)-4-oxobutanoic acid
  MOD_RES 11: note = O-(tert-butyl)-L-serine
  MOD_RES 12: note = O-(tert-butyl)-L-serine
  MOD_RES 13: note = (S)-2-amino-3-(4-(tert-butoxy)phenyl)propanoic acid
  MOD_RES 15: note = (S)-2-amino-5-(tert-butoxy)-5-oxopentanoic acid
  MOD_RES 17: note = N5-trityl-L-glutamine
HXEGTFTSDVSSYLEGQAA

SEQ ID NO: 149 - protein, 18 aa, synthetic construct
  REGION 1..18: note = synthetic peptide
  MOD_RES 1: note = N-alpha-(tert-butoxycarbonyl)-N-tau-trityl-L-histid ine
  MOD_RES 3: note = (S)-2-amino-5-(tert-butoxy)-5-oxopentanoic acid
  MOD_RES 5: note = O-(tert-butyl)-L-threonine
  MOD_RES 7: note = O-(tert-butyl)-L-threonine
  MOD_RES 8: note = O-(tert-butyl)-L-serine
  MOD_RES 9: note = (S)-2-amino-4-(tert-butoxy)-4-oxobutanoic acid
  MOD_RES 11: note = O-(tert-butyl)-L-serine
  MOD_RES 12: note = O-(tert-butyl)-L-serine
  MOD_RES 13: note = (S)-2-amino-3-(4-(tert-butoxy)phenyl)propanoic acid
  MOD_RES 15: note = (S)-2-amino-5-(tert-butoxy)-5-oxopentanoic acid
  MOD_RES 17: note = N5-trityl-L-glutamine
HAEGTFTSDVSSYLEGQA

SEQ ID NO: 150 - protein, 18 aa, synthetic construct
  REGION 1..18: note = synthetic peptide
  MOD_RES 1: note = N-alpha-(tert-butoxycarbonyl)-N-tau-trityl-L-histid ine
  MOD_RES 2: note = Aib
  MOD_RES 3: note = (S)-2-amino-5-(tert-butoxy)-5-oxopentanoic acid
  MOD_RES 5: note = O-(tert-butyl)-L-threonine
  MOD_RES 7: note = O-(tert-butyl)-L-threonine
  MOD_RES 8: note = O-(tert-butyl)-L-serine
  MOD_RES 9: note = (S)-2-amino-4-(tert-butoxy)-4-oxobutanoic acid
  MOD_RES 11: note = O-(tert-butyl)-L-serine
  MOD_RES 12: note = O-(tert-butyl)-L-serine
  MOD_RES 13: note = (S)-2-amino-3-(4-(tert-butoxy)phenyl)propanoic acid
  MOD_RES 15: note = (S)-2-amino-5-(tert-butoxy)-5-oxopentanoic acid
  MOD_RES 17: note = N5-trityl-L-glutamine
HXEGTFTSDVSSYLEGQA

SEQ ID NO: 151 - protein, 17 aa, synthetic construct
  REGION 1..17: note = synthetic peptide
  MOD_RES 1: note = N-alpha-(tert-butoxycarbonyl)-N-tau-trityl-L-histid ine
  MOD_RES 3: note = (S)-2-amino-5-(tert-butoxy)-5-oxopentanoic acid
  MOD_RES 5: note = O-(tert-butyl)-L-threonine
  MOD_RES 7: note = O-(tert-butyl)-L-threonine
  MOD_RES 8: note = O-(tert-butyl)-L-serine
  MOD_RES 9: note = (S)-2-amino-4-(tert-butoxy)-4-oxobutanoic acid
  MOD_RES 11: note = O-(tert-butyl)-L-serine
  MOD_RES 12: note = O-(tert-butyl)-L-serine
  MOD_RES 13: note = (S)-2-amino-3-(4-(tert-butoxy)phenyl)propanoic acid
  MOD_RES 15: note = (S)-2-amino-5-(tert-butoxy)-5-oxopentanoic acid
HAEGTFTSDVSSYLEGQ

SEQ ID NO: 152 - protein, 17 aa, synthetic construct
  REGION 1..17: note = synthetic peptide
  MOD_RES 1: note = N-alpha-(tert-butoxycarbonyl)-N-tau-trityl-L-histid ine
  MOD_RES 2: note = Aib
  MOD_RES 3: note = (S)-2-amino-5-(tert-butoxy)-5-oxopentanoic acid
  MOD_RES 5: note = O-(tert-butyl)-L-threonine
  MOD_RES 7: note = O-(tert-butyl)-L-threonine
  MOD_RES 8: note = O-(tert-butyl)-L-serine
  MOD_RES 9: note = (S)-2-amino-4-(tert-butoxy)-4-oxobutanoic acid
  MOD_RES 11: note = O-(tert-butyl)-L-serine
  MOD_RES 12: note = O-(tert-butyl)-L-serine
  MOD_RES 13: note = (S)-2-amino-3-(4-(tert-butoxy)phenyl)propanoic acid
  MOD_RES 15: note = (S)-2-amino-5-(tert-butoxy)-5-oxopentanoic acid
  MOD_RES 17: note = N5-trityl-L-glutamine
HXEGTFTSDVSSYLEGQ

SEQ ID NO: 153 - protein, 17 aa, synthetic construct
  REGION 1..17: note = synthetic peptide
  MOD_RES 1: note = N-alpha-(tert-butoxycarbonyl)-N-tau-trityl-L-histid ine
  MOD_RES 2: note = Aib
  MOD_RES 3: note = (S)-2-amino-5-(tert-butoxy)-5-oxopentanoic acid
  MOD_RES 5: note = O-(tert-butyl)-L-threonine
  MOD_RES 7: note = O-(tert-butyl)-L-threonine
  MOD_RES 8: note = O-(tert-butyl)-L-serine
  MOD_RES 9: note = (S)-2-amino-4-(tert-butoxy)-4-oxobutanoic acid
  MOD_RES 11: note = O-(tert-butyl)-L-serine
  MOD_RES 12: note = O-(tert-butyl)-L-serine
  MOD_RES 13: note = (S)-2-amino-3-(4-(tert-butoxy)phenyl)propanoic acid
  MOD_RES 15: note = (S)-2-amino-5-(tert-butoxy)-5-oxopentanoic acid
HXEGTFTSDVSSYLEGQ

SEQ ID NO: 154 - skipped

SEQ ID NO: 155 - protein, 5 aa, synthetic construct
  REGION 1..5: note = synthetic peptide
  MOD_RES 1: note = (S)-2-amino-5-(tert-butoxy)-5-oxopentanoic acid
  MOD_RES 3: note = N5-trityl-L-glutamine
EGQAA

SEQ ID NO: 156 - protein, 14 aa, synthetic construct
  REGION 1..14: note = synthetic peptide
  MOD_RES 1: note = N-alpha-(tert-butoxycarbonyl)-N-tau-trityl-L-histid ine
  MOD_RES 3: note = (S)-2-amino-5-(tert-butoxy)-5-oxopentanoic acid
  MOD_RES 5: note = O-(tert-butyl)-L-threonine
  MOD_RES 7: note = O-(tert-butyl)-L-threonine
  MOD_RES 8: note = O-(tert-butyl)-L-serine
  MOD_RES 9: note = (S)-2-amino-4-(tert-butoxy)-4-oxobutanoic acid
  MOD_RES 11: note = O-(tert-butyl)-L-serine
  MOD_RES 12: note = O-(tert-butyl)-L-serine
  MOD_RES 13: note = (S)-2-amino-3-(4-(tert-butoxy)phenyl)propanoic acid
HAEGTFTSDVSSYL

SEQ ID NO: 157 - protein, 14 aa, synthetic construct
  REGION 1..14: note = synthetic peptide
  MOD_RES 1: note = N-alpha-(tert-butoxycarbonyl)-N-tau-trityl-L-histid ine
  MOD_RES 2: note = Aib
  MOD_RES 3: note = (S)-2-amino-5-(tert-butoxy)-5-oxopentanoic acid
  MOD_RES 5: note = O-(tert-butyl)-L-threonine
  MOD_RES 7: note = O-(tert-butyl)-L-threonine
  MOD_RES 8: note = O-(tert-butyl)-L-serine
  MOD_RES 9: note = (S)-2-amino-4-(tert-butoxy)-4-oxobutanoic acid
  MOD_RES 11: note = O-(tert-butyl)-L-serine
  MOD_RES 12: note = O-(tert-butyl)-L-serine
  MOD_RES 13: note = (S)-2-amino-3-(4-(tert-butoxy)phenyl)propanoic acid
HXEGTFTSDVSSYL

SEQ ID NO: 158 - protein, 10 aa, synthetic construct
  REGION 1..10: note = synthetic peptide
  MOD_RES 1: note = (S)-2-amino-5-(tert-butoxy)-5-oxopentanoic acid
  MOD_RES 5: note = 1-(tert-butoxycarbonyl)-L-tryptophan
  MOD_RES 8: note = N-omega-((2,2,4,6,7-pentamethyl-2,3-dihydrobenzofur an-5-yl)sulfonyl)-L-arginine
  MOD_RES 10: note = N-omega-((2,2,4,6,7-pentamethyl-2,3-dihydrobenzofur an-5-yl)sulfonyl)-L-arginine
EFIAWLVRGR

SEQ ID NO: 159 - protein, 6 aa, synthetic construct
  REGION 1..6: note = synthetic peptide
  MOD_RES 1: note = (S)-2-amino-5-(tert-butoxy)-5-oxopentanoic acid
  MOD_RES 3: note = N5-trityl-L-glutamine
  MOD_RES 6: note = Lys(N-(17-carboxy-1-oxoheptadecyl)-L-gamma-glutamyl -2-[2-(2-amino ethoxy)ethoxy]acetyl-2-[2-(2-aminoethoxy)ethoxy]acetyl)
EGQAAK

SEQ ID NO: 160 - protein, 16 aa, synthetic construct
  REGION 1..16: note = synthetic peptide
  MOD_RES 1: note = (S)-2-amino-5-(tert-butoxy)-5-oxopentanoic acid
  MOD_RES 3: note = N5-trityl-L-glutamine
  MOD_RES 6: note = Lys(N-(17-carboxy-1-oxoheptadecyl)-L-gamma-glutamyl -2-[2-(2-amino ethoxy)ethoxy]acetyl-2-[2-(2-aminoethoxy)ethoxy]acetyl)
  MOD_RES 7: note = (S)-2-amino-5-(tert-butoxy)-5-oxopentanoic acid
  MOD_RES 11: note = 1-(tert-butoxycarbonyl)-L-tryptophan
  MOD_RES 14: note = N-omega-((2,2,4,6,7-pentamethyl-2,3-dihydrobenzofur an-5-yl)sulfonyl)-L-arginine
  MOD_RES 16: note = N-omega-((2,2,4,6,7-pentamethyl-2,3-dihydrobenzofur an-5-yl)sulfonyl)-L-arginine
EGQAAKEFIAWLVRGR

SEQ ID NO: 161 - protein, 12 aa, synthetic construct
  REGION 1..12: note = synthetic peptide
  MOD_RES 1: note = Lys(N-(17-carboxy-1-oxoheptadecyl)-L-gamma-glutamyl -2-[2-(2-amino ethoxy)ethoxy]acetyl-2-[2-(2-aminoethoxy)ethoxy]acetyl)
  MOD_RES 2: note = (S)-2-amino-5-(tert-butoxy)-5-oxopentanoic acid
  MOD_RES 6: note = 1-(tert-butoxycarbonyl)-L-tryptophan
  MOD_RES 9: note = N-omega-((2,2,4,6,7-pentamethyl-2,3-dihydrobenzofur an-5-yl)sulfonyl)-L-arginine
  MOD_RES 11: note = N-omega-((2,2,4,6,7-pentamethyl-2,3-dihydrobenzofur an-5-yl)sulfonyl)-L-arginine
  MOD_RES 12: note = Gly-O-carboxy protecting group
KEFIAWLVRGRG

SEQ ID NO: 162 - protein, 13 aa, synthetic construct
  REGION 1..13: note = synthetic peptide
  MOD_RES 2: note = Lys(N-(17-carboxy-1-oxoheptadecyl)-L-gamma-glutamyl -2-[2-(2-amino ethoxy)ethoxy]acetyl-2-[2-(2-aminoethoxy)ethoxy]acetyl)
  MOD_RES 3: note = (S)-2-amino-5-(tert-butoxy)-5-oxopentanoic acid
  MOD_RES 7: note = 1-(tert-butoxycarbonyl)-L-tryptophan
  MOD_RES 10: note = N-omega-((2,2,4,6,7-pentamethyl-2,3-dihydrobenzofur an-5-yl)sulfonyl)-L-arginine
  MOD_RES 12: note = N-omega-((2,2,4,6,7-pentamethyl-2,3-dihydrobenzofur an-5-yl)sulfonyl)-L-arginine
  MOD_RES 13: note = Gly-O-carboxy protecting group
AKEFIAWLVRGRG

SEQ ID NO: 163 - protein, 14 aa, synthetic construct
  REGION 1..14: note = synthetic peptide
  MOD_RES 3: note = Lys(N-(17-carboxy-1-oxoheptadecyl)-L-gamma-glutamyl -2-[2-(2-amino ethoxy)ethoxy]acetyl-2-[2-(2-aminoethoxy)ethoxy]acetyl)
  MOD_RES 4: note = (S)-2-amino-5-(tert-butoxy)-5-oxopentanoic acid
  MOD_RES 8: note = 1-(tert-butoxycarbonyl)-L-tryptophan
  MOD_RES 11: note = N-omega-((2,2,4,6,7-pentamethyl-2,3-dihydrobenzofur an-5-yl)sulfonyl)-L-arginine
  MOD_RES 13: note = N-omega-((2,2,4,6,7-pentamethyl-2,3-dihydrobenzofur an-5-yl)sulfonyl)-L-arginine
  MOD_RES 14: note = Gly-O-carboxy protecting group
AAKEFIAWLVRGRG

SEQ ID NO: 164 - protein, 15 aa, synthetic construct
  REGION 1..15: note = synthetic peptide
  MOD_RES 1: note = N5-trityl-L-glutamine
  MOD_RES 4: note = Lys(N-(17-carboxy-1-oxoheptadecyl)-L-gamma-glutamyl -2-[2-(2-amino ethoxy)ethoxy]acetyl-2-[2-(2-aminoethoxy)ethoxy]acetyl)
  MOD_RES 5: note = (S)-2-amino-5-(tert-butoxy)-5-oxopentanoic acid
  MOD_RES 9: note = 1-(tert-butoxycarbonyl)-L-tryptophan
  MOD_RES 12: note = N-omega-((2,2,4,6,7-pentamethyl-2,3-dihydrobenzofur an-5-yl)sulfonyl)-L-arginine
  MOD_RES 14: note = N-omega-((2,2,4,6,7-pentamethyl-2,3-dihydrobenzofur an-5-yl)sulfonyl)-L-arginine
  MOD_RES 15: note = Gly-O-carboxy protecting group
QAAKEFIAWLVRGRG

SEQ ID NO: 165 - protein, 16 aa, synthetic construct
  REGION 1..16: note = synthetic peptide
  MOD_RES 1: note = (S)-2-amino-5-(tert-butoxy)-5-oxopentanoic acid
  MOD_RES 3: note = N5-trityl-L-glutamine
  MOD_RES 6: note = Lys(N-(17-carboxy-1-oxoheptadecyl)-L-gamma-glutamyl -2-[2-(2-amino ethoxy)ethoxy]acetyl-2-[2-(2-aminoethoxy)ethoxy]acetyl)
  MOD_RES 7: note = (S)-2-amino-5-(tert-butoxy)-5-oxopentanoic acid
  MOD_RES 11: note = 1-(tert-butoxycarbonyl)-L-tryptophan
  MOD_RES 14: note = N-omega-((2,2,4,6,7-pentamethyl-2,3-dihydrobenzofur an-5-yl)sulfonyl)-L-arginine
  MOD_RES 16: note = N-omega-((2,2,4,6,7-pentamethyl-2,3-dihydrobenzofur an-5-yl)sulfonyl)-L-arginine-O-carboxy protecting group
EGQAAKEFIAWLVRGR